(19) United States
(12) Patent Application Publication
(10) Pub. No.: US 2013/0053271 A1
(43) Pub. Date: Feb. 28, 2013

(54) Self-Assembled Bead-Based Multiplexed Assay For Antigen-Specific Antibodies

(71) Applicants: Gerard Zurawski, Midlothian, TX (US); Sandra Zurawski, Midlothian, TX (US)

(72) Inventors: Gerard Zurawski, Midlothian, TX (US); Sandra Zurawski, Midlothian, TX (US)

(73) Assignee: BAYLOR RESEARCH INSTITUTE, Dallas, TX (US)

(21) Appl. No.: 13/594,397

(22) Filed: Aug. 24, 2012

Related U.S. Application Data
(60) Provisional application No. 61/527,302, filed on Aug. 25, 2011.

(51) Int. Cl. C40B 30/04; G01N 33/573; G01N 33/566; G01N 21/64; G01N 21/78; C12Q 1/70; G01N 33/571; C12N 9/00; C12N 1/00; C07K 16/00; C07K 1/22; C07K 14/00; C07K 19/00; C07H 21/00; B01D 15/08; G01N 33/569; B82Y 15/00

(52) U.S. Cl. 506/9; 435/7.1; 435/7.4; 436/501; 435/5; 435/183; 435/243; 530/387.1; 530/344; 530/413; 530/409; 536/25.4; 210/670; 977/773; 977/920; 977/902

(57) ABSTRACT

A flexible method for making multiplexed assays for antigen-specific antibody responses is disclosed herein. The method of the present invention comprises incubation of a substrate or a set of beads coated with a dockerin or a cohesin protein with a cohesin or dockerin-antigen fusion proteins to attach the fusion protein essentially irreversibly by non-covalent interaction. The present invention enables the use of one dockerin- or cohesin-coated bead for multiple sets of cohesin- or dockerin-antigens, obviating the need to directly chemically couple new antigens to new beads.

### CROSS-REFERENCE TO RELATED APPLICATIONS

This application claims priority to U.S. Provisional Application Ser. No. 61/527,302 filed Aug. 25, 2011, the entire contents of which are incorporated herein by reference.

### STATEMENT OF FEDERALLY FUNDED RESEARCH

This invention was made with government support under Grant No. 1U19AI057234-010000 awarded by the NIH the government has certain rights in the invention.

### TECHNICAL FIELD OF THE INVENTION

The present invention relates in general to the field of novel tools for molecular analysis, and more particularly, to the design, manufacture and use of beads that include either cohesin or dockerin for multiplex analysis.

### REFERENCE TO A SEQUENCE LISTING

The present application includes a Sequence Listing which has been submitted in ASCII format via EFS-Web and is hereby incorporated by reference in its entirety. Said ASCII copy, created on Aug. 21, 2012, is named BHCS 1137 Sequence Listing.txt and is 299 KB in size.

### BACKGROUND OF THE INVENTION

The present invention relates to methods of making a multiplexed assays for antigen-specific antibody responses. Affinity chromatography is one separation technique for isolating biologically active compounds. The separation is determined by a variety of factors including the binding constant, selectivity of the column, good column retention, the capacity of the column, and elution conditions. For example, a high affinity between the ligand and the biomolecule may require harsh elution conditions (such as low/high pH or very high salt concentrations), which may lead to unfolding or denaturation in the case of a recombinant protein, whereas an overly weak affinity may be insufficient for efficient retention on the column. For example, micro beads in distinct sets are be coated with a reagent specific to a particular bioassay, allowing the capture and detection of specific analytes from a sample. The beads are directly chemically couple to a micro bead and new antigens must be directly chemically couple to new beads.

For example, U.S. Patent Application Publication No. 2010/0062451, entitled “Bead-Ligand-Nascent Protein Complexes” discloses bad-ligand-nascent protein complexes, and method of creating and detecting a bead-ligand-nascent protein complexes, are described. PCR-amplified product which is attached to a surface, e.g. of a bead, is used to generate nascent protein, which in turn is captured on the bead and detected, e.g. by fluorescence.

U.S. Patent Application Publication No. 2005/0106700 discloses use of C-terminal and N-terminal dockerin fusions in purification of target proteins on affinity columns.

International Patent Application No. WO 2009/028532 discloses purification systems and methods using a dockerin polypeptide characterized in that the amino acid at the 14-position in the second sub-domain of a dockerin originating from Clostridium josui is substituted by another amino acid.

U.S. Patent Application Publication No. 2011/0151538, entitled “Affinity Purification by Cohesin-Dockerin Interaction” discloses truncated dockerin polypeptides, recombinant polypeptides and affinity systems having the truncated dockerin polypeptide, methods of generating same, and methods of use thereof to purify, isolate, and detect molecules of interest, where the solid substrate is cellulose with carbohydrate-binding module (CBM) and the protein bound. The entire contents of this application are incorporated herein by reference.

### BRIEF SUMMARY OF THE INVENTION

The present invention discloses a flexible method for making multiplexed assays for antigen-specific antibody responses by the use of one dockerin coated bead for multiple sets of cohesin-antigens, obviating the need to directly chemically couple new antigens to new beads. The present invention provides a very flexible method, and compositions for use in the same, for making multiplexed assays for antigen-specific antibody responses. In contrast to standard multiplex technology (such as Luminex™ or equivalents) in which color-coded micro beads of up to 500 distinct sets are coated with a reagent specific to a particular bioassay (allowing the capture and detection of specific analytes from a sample) the present invention takes advantage of the essentially irreversible binding between cohesin and dockerin proteins to add the flexibility to the beads. In the present invention, all beads in the set are first coated with a dockerin or cohesin domain containing protein. Then each bead set is simply incubated with cohesin-antigen fusion proteins, which attach essentially irreversibly by non-covalent interaction. Thus, one dockerin or cohesin coated bead set can be used for multiple sets of cohesin or dockerin-antigens, obviating the need to directly chemically couple new antigens to new beads.

In one embodiment, the present invention comprises a method for detecting, isolating, or purifying one or more analytes in a sample, in a matrix, from a mixture, or any combinations thereof comprising: obtaining a solid substrate comprising a first member of a cohesin-dockerin binding pair, wherein the first member is attached to a protein, an antibody, an antigen, a peptide, a toxin, a cytokine, an enzyme, a structural protein, an extracellular matrix protein, a cell, or fragments thereof, wherein the solid substrate is selected from the group consisting of a bead, a cell, an extracellular matrix, a fibrous matrix, a container, an affinity column, or any combinations thereof; providing a second member of the cohesin-dockerin binding pair, wherein the second member is present in the sample, the matrix, the mixture or any combinations thereof, wherein the second member is capable of binding to one or more analytes to be detected, isolated, or purified from the sample, the matrix, the mixture or any combinations thereof; contacting the second member of the cohesin-dockerin binding pair with the sample, the matrix or the mixture suspected of having the analyte; and forming a complex on the substrate comprising the first and the second members of the cohesin-dockerin binding pairs and the analyte, wherein the presence of the analyte is detected. In one aspect, the method further comprises adding a detection reagent to the complex for determining presence or absence of the analyte, wherein the detection reagent comprises a secondary antibody, a radiolabel, a flurophore, a colorimetric reagent, or any combinations thereof. In another aspect, the analyte comprises a nucleic acid (natural and unnatural, aptamers), carbohydrates, polysaccharides, peptides, proteins or peptoids (natural and unnatural), minerals, vitamins and lipids. In another aspect, the first member comprises a dockerin domain and the second member comprises a cohesin domain bound to the analyte. In another aspect, the first member comprises a cohesin domain and the second member comprises a dockerin domain bound to the analyte. In another aspect, the second member comprises a cohesin or a dockerin domain and the analyte forms a fusion protein with the cohesin or dockerin domain. In another aspect, the method is carried out in a container that comprises a beaker, a flask, a cylinder, a test tube, a centrifugation tube, a petri dish, a culture dish, or a multi-well plate. In another aspect, the substrate comprises one or more sets of beads, wherein the beads comprise nanospheres, nanoparticles, microspheres, microparticles, nanobeads, microbeads, beads, polystyrene beads, latex particles, latex beads, fluorescent beads, fluorescent particles, colored particles, or colored beads. In another aspect, the one or more sets of beads comprise polymeric beads, wherein the polymers are selected from the group consisting of carbohydrate-based polymers, polyaliphatic alcohols, poly(vinyl) polymers, polyacrylic acids, polyorganic acids, polyamino acids, co-polymers, block co-polymers, tertpolymers, polyethers, naturally occurring polymers, polyimids, surfactants, polyesters, branched polymers, cyclo-polymers, polyaldehydes and mixtures thereof, brominated polystyrene, polyacrylic acid, polyacrylonitrile, polyamide, polyacrylamide, polyacrolein, polybutadiene, polycaprolactone, polyester, polyethylene, polyethylene terephthalate, polydimethylsiloxane, polyisoprene, polyurethane, polyvinylacetate, polyvinylchloride, polyvinylpyridine, polyvinylbenzylchloride, polyvinyltoluene, polyvinylidene chloride, polydivinylbenzene, polymethylmethacrylate, polylactide, polyglycolide, poly(lactide-co-glycolide), polyanhydride, polyorthoester, polyphosphazene, polyphosophaze,poly-(styrene-co-vinylbenzyl chloride-co-acrylic acid) (85:10:5 molar ratio), poly(styrene-co-acrylic acid) (99:1 molar ratio), poly(styrene-co-methacrylic acid) (90:10 molar ratio), poly(styrene-co-acrylic acid-co-m&p-divinylbenzene) (89:10:1 molar ratio), poly-(styrene-co-2-carboxyethyl acrylate) (90:10 molar ratio), poly(methyl methacrylate-co-acrylic acid) (70:30 molar ratio) and poly(styrene-co-butyl acrylate-co-methacrylic acid)(45:45:10 weight ratio) synthetic polymers polystyrene, polyacrylamide, polyacrylate, latex, and any combinations or modifications thereof. In another aspect, the one or more analytes comprise antigens, antibodies, autoantibodies, peptides, proteins, nucleic acid sequences, and/or enzymes, wherein the antigens comprise one or more bacterial, viral, fungal, mycoplasmal, rickettsial, chlamydial, or protozoal antigens. In another aspect, the dockerin may be a substituted dockerin, a truncated dockerin, a modified dockerin, or any combinations thereof. In another aspect, the cohesin may be a substituted cohesin, a truncated cohesin, a modified cohesin, or any combinations thereof. In another aspect, the cohesin is derived or isolated from Clostridium thermocellum, C. cellulolyticum, C. cellulovorans, C. papyrosolvens, and Clostridium cellobioparum, C. papyrosolvens, C. josui, Acetivibrio cellulolyticus, Bacteroides cellulosolvens, R. flavefaciens, Ruminococcus albus, Archaeoglobus fulgidus protein, cellulosomal cohesin domain.

In one embodiment, the present invention comprises a multiplex bead based method for detecting, isolating, or purifying one or more analytes in a sample, in a matrix, from a mixture, or any combinations thereof comprising: providing one, a plurality, or a set of beads comprising nanospheres, nanoparticles, microspheres, microparticles, nanobeads, microbeads, beads, polystyrene beads, latex particles, latex beads, fluorescent beads, fluorescent particles, colored particles, or colored beads; attaching a first member of a cohesin-dockerin binding pair to the beads, wherein the first member is attached to a protein, an antibody, an antigen, a peptide, a toxin, a cytokine, an enzyme, a structural protein, an extracellular matrix protein, a cell, or fragments thereof; providing at least one second member of the cohesin-dockerin binding pair attached to the second member is attached to the one or more analytes to be detected, isolated, or purified from the sample, the matrix, the mixture or any combinations thereof; contacting the first member of the cohesin-dockerin binding pair with the sample, the matrix or the mixture comprising the second cohesin-dockerin binding pair; and forming a complex comprising at least one second member of the cohesin-dockerin binding pair bound irreversibly in a non-covalent manner to the at least one first member of the cohesin-dockerin binding pair. In one aspect, the method further comprises adding a detection reagent to the complex for determining presence or absence of the analyte, wherein the detection reagent comprises a secondary antibody, a radiolabel, a flurophore, a colorimetric reagent, or any combinations thereof; releasing the second member comprising the analyte from the complex by one or more physical or chemical methods; and isolating the analyte from a mixture comprising cohesin, dockerin, proteins, antigens, peptides, antibodies, or any combinations thereof. In another aspect, the analyte comprises a nucleic acid (natural and unnatural, aptamers), carbohydrates, polysaccharides, peptides, proteins or peptoids (natural and unnatural), minerals, vitamins and lipids. In another aspect, the first member comprises a dockerin domain and the second member comprises a cohesin domain bound to the analyte. In another aspect, the first member comprises a cohesin domain and the second member comprises a dockerin domain bound to the analyte. In another aspect, the second member comprises a cohesin or a dockerin domain and the analyte forms a fusion protein with the cohesin or dockerin domain. In another aspect, the one or more sets of beads comprise polymeric beads, wherein the polymers are selected from the group consisting of carbohydrate-based polymers, polyaliphatic alcohols, poly(vinyl) polymers, polyacrylic acids, polyorganic acids, polyamino acids, co-polymers, block co-polymers, tertpolymers, polyethers, naturally occurring polymers, polyimide, surfactants, polyesters, branched polymers, cyclo-polymers, polyaldehydes and mixtures thereof, brominated polystyrene, polyacrylic acid, polyacrylonitrile, polyamide, polyacrylamide, polyacrolein, polybutadiene, polycaprolactone, polyester, polyethylene, polyethylene terephthalate, polydimethylsiloxane, polyisoprene, polyurethane, polyvinylacetate, polyvinylchloride, polyvinylpyridine, polyvinylbenzylchloride, polyvinyltoluene, polyvinylidene chloride, polydivinylbenzene, polymethylmethacrylate, polylactide, polyglycolide, poly(lactide-co-glycolide), polyanhydride, polyorthoester, polyphosphazene, polyphosophaze,poly-(styrene-co-vinylbenzyl chloride-co-acrylic acid) (85:10:5 molar ratio), poly(styrene-co-acrylic acid) (99:1 molar ratio), poly(styrene-co-methacrylic acid) (90:10 molar ratio), poly(styrene-co-acrylic acid-co-m&p-divinylbenzene) (89:10:1 molar ratio), poly-(styrene-co-2-carboxyethyl acrylate) (90:10 molar ratio), poly(methyl methacrylate-co-acrylic acid) (70:30 molar ratio) and poly(styrene-co-butyl acrylate-co-methacrylic acid)(45:45:10 weight ratio) synthetic polymers polystyrene, polyacrylamide, polyacrylate, latex, or any combinations or modifications thereof. In another aspect, the one or more analytes comprise antigens, antibodies, autoantibodies, peptides, proteins, nucleic acid sequences, and/or enzymes, wherein the antigens comprise one or more bacterial, viral, fungal, mycoplasmal, rickettsial, chlamydial, and/or protozoal antigens. In another aspect, the dockerin is selected from a Domain I dockerin, a Domain II dockerin, a Domain III dockerin, a substituted dockerin, a truncated dockerin, a modified dockerin, or any combinations thereof. In another aspect, the cohesin may be a Type I cohesin, a Type II cohesin, a Type III cohesin, a substituted cohesin, a truncated cohesin, a modified cohesin, or any combinations thereof.

Another embodiment of the invention is an assay system comprising: a substrate and at least one attached or immobilized dockerin or cohesin binding domain bound to the substrate, and a dockerin or cohesin binding pair is attached to a protein, an antibody, an antigen, a peptide, a toxin, a cytokine, an enzyme, a structural protein, an extracellular matrix protein, a cell, or fragments thereof. In one aspect, the substrate comprises one or more beads. In another aspect, the substrate comprises nanospheres, nanoparticles, microspheres, microparticles, nanobeads, microbeads, beads, polystyrene beads, latex particles, latex beads, fluorescent beads, fluorescent particles, colored particles, and colored beads. In another aspect, the analyte comprises a nucleic acid (natural and unnatural, aptamers), carbohydrates, polysaccharides, peptides, proteins or peptoids (natural and unnatural), minerals, vitamins and lipids. In another aspect, the first member comprises a dockerin domain and the second member comprises a cohesin domain bound to the analyte. In another aspect, the first member comprises a cohesin domain and the second member comprises a dockerin domain bound to the analyte. In another aspect, the second member comprises a cohesin or a dockerin domain and the analyte forms a fusion protein with the cohesin or dockerin domain.

In one embodiment, the present invention comprises a bead based assay system comprising one or more or a set of beads at least one attached or immobilized dockerin or cohesin binding domain, wherein the dockerin or cohesin binding domain may be attached to a protein, an antibody, an antigen, a peptide, a toxin, a cytokine, an enzyme, a structural protein, an extracellular matrix protein, a cell, or fragments thereof. In one aspect, the one or more beads comprise nanospheres, nanoparticles, microspheres, microparticles, nanobeads, microbeads, beads, polystyrene beads, latex particles, latex beads, fluorescent beads, fluorescent particles, colored particles, or colored beads. In another aspect, the analyte comprises a nucleic acid (natural and unnatural, aptamers), carbohydrates, polysaccharides, peptides, proteins or peptoids (natural and unnatural), minerals, vitamins or lipids. In another aspect, the first member comprises a dockerin domain and the second member comprises a cohesin domain bound to the analyte. In another aspect, the first member comprises a cohesin domain and the second member comprises a dockerin domain bound to the analyte. In another aspect, the second member comprises a cohesin or a dockerin domain and the analyte forms a fusion protein with the cohesin or dockerin domain.

In one embodiment, the present invention comprises a method for performing an immunoassay on a bead surface for the simultaneous detection of more than one analyte from a sample, a matrix, or a mixture comprising the steps of: providing one or more beads or sets of beads on a substrate, wherein the substrate is selected from the group consisting of a beaker, a flask, a cylinder, a test tube, a centrifugation tube, a petri dish, a culture dish, and a multi-well plate, or any combinations or modifications thereof attaching or immobilizing at least two dockerin binding domain to a surface of the beads, wherein the dockerin binding domain may be attached to a protein, an antibody, an antigen, a peptide, a toxin, a cytokine, an enzyme, a structural protein, an extracellular matrix protein, a cell, or fragments thereof; contacting the beads with the attached or immobilized dockerin binding domains with the sample, the matrix or the mixture comprising the multiple analytes of interest, wherein the multiple analytes are attached to one or more cohesin fusion proteins; forming more than one complexes comprising the multiple cohesin fusion protein attached analyte with the multiple dockerin binding domains on the surface of the one or more beads; and adding multiple detecting reagents or labels to the beads, wherein each of the detecting regent or label is specific and binds to the multiple analytes suspected of being present in the sample, matrix, or the mixture, and the detection reagents comprise a secondary antibody, a radiolabel, a fluorophore, a colorimetric reagent, or any combinations thereof; and detecting the presence or absence of the multiple analytes be reading, monitoring, or measuring a signal emitted by the bound detection reagent or label and the analyte. In one aspect, the method further comprises the optional steps of: performing one or more wash steps with a suitable buffer or water at one or in between different steps of the immunoassay; generating a calibration curve or a standard curve for determination of a concentration or an amount of the multiple analytes in the sample, the matrix, or the mixture by performing the immunoassay using one or more pure analytes or standards; releasing the cohesin fusion protein attached to the analyte from the complex by one or more physical or chemical methods; and isolating the analyte from a mixture comprising cohesin, dockerin, proteins, antigens, peptides, antibodies, or any combinations thereof. In one aspect, the substrate comprises one or more sets of beads, wherein the beads comprise nanospheres, nanoparticles, microspheres, microparticles, nanobeads, microbeads, beads, polystyrene beads, latex particles, latex beads, fluorescent beads, fluorescent particles, colored particles, or colored beads. In another aspect, the one or more sets of beads comprise polymeric beads, wherein the polymers are selected from the group consisting of carbohydrate-based polymers, polyaliphatic alcohols, poly(vinyl) polymers, polyacrylic acids, polyorganic acids, polyamino acids, co-polymers, block co-polymers, tertpolymers, polyethers, naturally occurring polymers, polyimide, surfactants, polyesters, branched polymers, cyclo-polymers, polyaldehydes and mixtures thereof, brominated polystyrene, polyacrylic acid, polyacrylonitrile, polyamide, polyacrylamide, polyacrolein, polybutadiene, polycaprolactone, polyester, polyethylene, polyethylene terephthalate, polydimethylsiloxane, polyisoprene, polyurethane, polyvinylacetate, polyvinylchloride, polyvinylpyridine, polyvinylbenzylchloride, polyvinyltoluene, polyvinylidene chloride, polydivinylbenzene, polymethylmethacrylate, polylactide, polyglycolide, poly(lactide-co-glycolide), polyanhydride, polyorthoester, polyphosphazene, polyphosophaze,poly-(styrene-co-vinylbenzyl chloride-co-acrylic acid) (85:10:5 molar ratio), poly(styrene-co-acrylic acid) (99:1 molar ratio), poly(styrene-co-methacrylic acid) (90:10 molar ratio), poly(styrene-co-acrylic acid-co-m&p-divinylbenzene) (89:10:1 molar ratio), poly-(styrene-co-2-carboxyethyl acrylate) (90:10 molar ratio), poly(methyl methacrylate-co-acrylic acid) (70:30 molar ratio) and poly(styrene-co-butyl acrylate-co-methacrylic acid)(45:45:10 weight ratio) synthetic polymers polystyrene, polyacrylamide, polyacrylate, latex, and any combinations or modifications thereof. In another aspect, the beads may be free flowing beads or may be attached to the solid substrate. In another aspect, the one or more analytes comprise antigens, antibodies, autoantibodies, peptides, proteins, nucleic acid sequences, enzymes, or small molecules wherein the antigens comprise one or more bacterial, viral, fungal, mycoplasmal, rickettsial, chlamydial, or protozoal antigens. In another aspect, the dockerin may be a substituted dockerin, a truncated dockerin, a modified dockerin, or any combinations thereof. In another aspect, the cohesin may be a substituted cohesin, a truncated cohesin, a modified cohesin, or any combinations thereof. In another aspect, the cohesin is derived or isolated from Clostridium thermocellum, C. cellulolyticum, C. cellulovorans, C. papyrosolvens, and Clostridium cellobioparum, C. papyrosolvens, C. josui, Acetivibrio cellulolyticus, Bacteroides cellulosolvens, R. flavefaciens, Ruminococcus albus, Archaeoglobus fulgidus protein, cellulosomal cohesin domain.

In one embodiment, the present invention comprises an affinity purification method utilizing one or more beads comprising the steps of: providing one or more beads or bead sets on a substrate, wherein the substrate is selected from the group consisting of a beaker, a flask, a cylinder, a test tube, a centrifugation tube, a petri dish, a culture dish, and a multi-well plate, an column and any combinations or modifications thereof; attaching or immobilizing at least one dockerin binding domain to a surface of the beads, wherein the dockerin binding domain may be attached to a protein, an antibody, an antigen, a peptide, a toxin, a cytokine, an enzyme, a structural protein, an extracellular matrix protein, a cell, or fragments thereof; contacting the beads by flowing or pumping a sample, a cellular mixture, a fermentation medium, a cell extract, or any combinations thereof comprising one or more analytes to be purified, wherein at least one of the analyte to be purified is coupled or attached to a cohesin fusion protein; binding the analyte comprising attached cohesin fusion protein with the one or more beads comprising at least one dockerin binding domain to form a complex, wherein the binding generates a flow through comprising one or more undesirable materials or materials from which an isolation of the analyte is desired; and releasing the desired analyte from the complex by one or a combination of physical or chemical methods, wherein the methods comprise a change in ionic strengths, addition of EDTA, removal of Ca^{2 +} from the medium, pH, temperature, or any combinations thereof. In another aspect, the one or more beads are immobilized to a solid substrate or a column packing material. In another aspect, the one or more beads or bead sets are packed in a column. In another aspect, the one or more beads are polymeric beads and comprise nanospheres, nanoparticles, microspheres, microparticles, nanobeads, microbeads, beads, polystyrene beads, latex particles, latex beads, fluorescent beads, fluorescent particles, colored particles, or colored beads. In another aspect, the analyte to be purified comprises one or more of antigens, antibodies, autoantibodies, peptides, proteins, fusion proteins, nucleic acid sequences, or enzymes, or any combination thereof wherein the antigens comprise one or more bacterial, viral, fungal, mycoplasmal, rickettsial, chlamydial, or protozoal antigens. In another aspect, the dockerin may be a substituted dockerin, a truncated dockerin, a modified dockerin, or any combinations thereof. In another aspect, the cohesin may be a substituted cohesin, a truncated cohesin, a modified cohesin, or any combinations thereof. In another aspect, the cohesin is derived or isolated from Clostridium thermocellum, C. cellulolyticum, C. cellulovorans, C. papyrosolvens, and Clostridium cellobioparum, C. papyrosolvens, C. josui, Acetivibrio cellulolyticus, Bacteroides cellulosolvens, R. flavefaciens, Ruminococcus albus, Archaeoglobus fulgidus protein, or a cellulosomal cohesin domain.

### BRIEF DESCRIPTION OF THE SEVERAL VIEWS OF THE DRAWINGS

For a more complete understanding of the features and advantages of the present invention, reference is now made to the detailed description of the invention along with the accompanying figures and in which:

FIGS. 1A and 1B show that Cohesin and dockerin are protein modules from cellulose-degrading bacteria that bind non-covalently with very high affinity (K_{D}˜30 pM): FIG. 1A A set of up to 100 different fluorescent microspheres (e.g., Luminex® beads) are cross-linked chemically to a cellulose-binding domain fused to dockerin (CBD-Doc). Each cohesin-antigen fusion protein is mixed to a CBD-Doc beads of a particular color (e.g., bead region 21). The beads are washed and pooled with CBD-Doc beads similarly coated with other desired cohesin-antigen fusion proteins, FIG. 1B The bead-antigen sets are incubated with a dilution series of each serum sample, washed, incubated with e.g., anti-human IgG-PE conjugate, washed again, then read in a flow cytometry instrument which measures fluorescence intensity associated with each bead color;

FIG. 2 is a comparison of serum anti-Gag p24 assays using the bead-based assay and standard ELISA. The three upper panels are serial dilutions of pre-immune serum (orange) and immune serum (blue) from three individuals assayed using Cohesin-Gag p24 coated beads. The three lower panels are the identical samples assayed using standard ELISA with Cohesin-Gag p24-coated plates. Consistently, the bead-based assay is 10-fold more sensitive;

FIG. 3 represents a serum anti-Influenza antigen-specific antibody assay using the bead-based multiplexed assay of the present invention. Dilutions of serum samples from four normal donors. This is a multiplexed assay with beads displaying antigens Ml (PR8), HA1-1 (PR8) and HA1-1 (HAS, avian H1N1 and Flu NP, avian H1N1). Beads displaying Cohesin alone are included to define the non-specific background. None of the donors had detectable antibodies binding to H1N1 avian influenza HA1-1;

FIG. 4 represents reagents for capture and detection of antigen-specific B cells. Beads displaying a Cohesin-antigen fusion protein (Ag-1) can be used to enrich antigen-specific B cells from ex vivo samples, while antigen tetramers assembled via streptavidin-phycoerythrin (SA-PE): biotin-CBD-Doc: Cohesin-antigen complex can be uses in flow cytometry to sort or quantify antigen-specific B cells. The antigen-bead complexes can also be assembled on biotin-coated Q dots of different, permitting the possible development of multiplexed flow analysis of several antigen-specific B cell categories. Furthermore, the combinatorial strategy of mixing tetramers for each specificity but with two different colors [Newell et al., Nature Methods 6, 497 - 499 (2009)] can be readily applied to our antigen tetramers to reduce background staining and increase the number of antigens that can be tested simultaneously;

FIG. 5 is an example of a multiplex bead-based assay of antigen-specific serum antibodies from a Influenza-infected non-human primate. Three different HA antigens are represented. As well as M1, M2 ectodomain, NP, and NA; and

FIGS. 6A-6C provide an example of a multiplex bead antigen-specific IgG assay: FIG. 6A a serum sample from an influenza-infected monkey was diluted and mixed with a bead set coated with cohesin fused to various Influenza proteins NPS, nuclear protein; HAl PR8, HA1 domain from hemagglutinin of the PR8 strain; HA1 SF, HAl domain from hemagglutinin of the swine flu strain (CAL04); HA3, HA1 domain from hemagglutinin a H3 Influenza strain; M2e, the ectodomain of the M2 protein from PR8; M1, matrix protein 1; Coh, Cohesin without antigen. FIGS. 6B and 6C compares the serum from the monkey before (FIG. 6B) and after vaccination (FIG. 6C) with a vaccine bearing HAl from PR8.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

As used herein, the term “analogs” extends to any functional chemical or recombinant equivalent of the peptides of the present invention, characterized, in a most preferred embodiment, by their possession of at least one of the abovementioned activities. The term “analog” is also used herein to extend to any amino acid derivative of the peptides as described hereinabove. Generally, an analog will possess in one embodiment at least 70% sequence identity, other embodiments can have at least 75, 80, 85, 90, 91, 92, 93, 94, 95, 96, 97, 98, 99, or 100% sequence identity. Percentage sequence identity can be determined by the skilled artisan.

As used herein, the term “epitope(s)” refer to a peptide or protein antigen that includes a primary, secondary or tertiary structure similar to an epitope located within any of a number of pathogen polypeptides encoded by the pathogen DNA or RNA. The level of similarity will generally be to such a degree that monoclonal or polyclonal antibodies directed against such polypeptides will also bind to, react with, or otherwise recognize, the peptide or protein antigen.

As used herein, the term “recombinant polypeptide” refers to a polypeptide that has been produced in a host cell which has been transformed or transfected with a nucleic acid encoding the polypeptide, or produces the polypeptide as a result of homologous recombination.

As used herein, the term “dockerin-cohesin pair” is used to denote the binding of the dockerin domain and the cohesin domain and it is understood that the dockerin domain, the cohesin domain or both may be truncated, substituted, modified, or altered. In addition, the embodiments are described in various ways but it is to be understood that the dockerin-cohesin pair can also be considered a cohesin-dockerin pair. It is to be understood that either the dockerin or the cohesin may be bound to the substrate and the corresponding pair may be conjugated to the binding agent to form a dockerin-cohesin pair.

Microarray, micro-bead and bead technologies of the present invention can be used as tools to conduct biological, chemical or biochemical analyses in a parallel, massively parallel or multiplexed fashion because of the large number of different compounds or substances that can be fabricated or deposited on the microarray substrate or beads. Micro-bead technologies are analogous to microarrays except that the features are spatially segregated on different beads or particles. The analysis can be formatted like a microarray, for example, with the beads arrayed or embedded on the surface or in wells of a device such as a microscope slide or plate. The analysis can alternatively be performed with the beads suspended in a solution for example. The working density of features for micro-bead technologies is potentially far greater than for microarrays, depending primarily on the minimum usable bead size and maximum usable bead concentration or density.

As used herein, the term “nanospheres” is used to denote particles in sizes ranging from about 10 nanometers (nm) to about 100,000 nm in diameter. Optimally preferred diameters are within about 10 and 1,000 nm, preferably within 200 and 500 nm. Polymeric microspheres used in this invention as carrier particles to which nanospheres are bound normally range in size from 0.01 to 1000 micrometers in diameter. Even though the microparticle can be of any size, the preferred size is 0.1-500 micrometers, more preferably 1-200 micrometers, and even more preferably 2-12 micrometers. The particles can be uniform (being about the same size) or of variable size such that the differences can be determined by size-dependent properties such as light scattering or optical refraction. Particles are made of any regularly shaped material. The preferred shape is spherical, however, particles of any other shape can be employed since this parameter is immaterial to the nature of the invention.

Usually these nanospheres as well as carrier particles are made of the same material such as polystyrene or latex. However, other polymeric materials are acceptable including polymers selected from the chemical group consisting of carbohydrate-based polymers, polyaliphatic alcohols, poly(vinyl) polymers, polyacrylic acids, polyorganic acids, polyamino acids, co-polymers, block co-polymers, tertpolymers, polyethers, naturally occurring polymers, polyimide, surfactants, polyesters, branched polymers, cyclo-polymers, polyaldehydes and mixtures thereof More specifically, brominated polystyrene, polyacrylic acid, polyacrylonitrile, polyamide, polyacrylamide, polyacrolein, polybutadiene, polycaprolactone, polyester, polyethylene, polyethylene terephthalate, polydimethylsiloxane, polyisoprene, polyurethane, polyvinylacetate, polyvinylchloride, polyvinylpyridine, polyvinylbenzylchloride, polyvinyltoluene, polyvinylidene chloride, polydivinylbenzene, polymethylmethacrylate, polylactide, polyglycolide, poly(lactide-co-glycolide), polyanhydride, polyorthoester, polyphosphazene, polyphosophaze, or combinations thereof are preferable. Representative combination polymers of which the polymeric particles are composed include for example poly-(styrene-co-vinylbenzyl chloride-co-acrylic acid) (85:10:5 molar ratio), poly(styrene-co-acrylic acid) (99:1 molar ratio), poly(styrene-co-methacrylic acid) (90:10 molar ratio), poly(styrene-co-acrylic acid-co-m&p-divinylbenzene) (89:10:1 molar ratio), poly-(styrene-co-2-carboxyethyl acrylate) (90:10 molar ratio), poly(methyl methacrylate-co-acrylic acid) (70:30 molar ratio) and poly(styrene-co-butyl acrylate-co-methacrylic acid)(45:45:10 weight ratio). Most of beads formed from synthetic polymers such as polystyrene, polyacrylamide, polyacrylate, or latex are now commercially available from numerous sources such as Bio-Rad Laboratories (Richmond, Calif.) and LKB Produkter (Stockholm, Sweden). Beads formed from natural macromolecules and particles such as agarose, crosslinked agarose, globulin, deoxyribose nucleic acid, and liposomes are commercially available from sources such as Bio-Rad Laboratories, Pharmacia (Piscataway, N.J.), and IBF (France). Beads formed from copolymers of polyacrylamide and agarose are commercially available from sources such as IBF and Pharmacia.

In general, the present invention provides the production of a plurality of substrates (e.g., beads) with different features such as antibodies, markers, binding agents, probes, targets or analytes for example, that can serve as delivery agents, purification mechanisms, isolation mechanisms, affinity matrix, multiplex arrays or similar functions. The different feature substances attached to the beads are typically produced off-line and can then be bound to beads in separate reactors, in a mechanical process of mixing solutions containing to form the dockerin-cohesin binding pair. This can be done in separate test tubes, vials or wells of a microtiter plate for example.

The present invention provides an affinity matrix system having a dockerin-cohesin pair bound to a substrate and configured to bind an antigen or other composition. The present invention provides a flexible method for making multiplexed assays for antigen-specific antibody responses. A standard Luminex technology uses color-coded micro beads in up to 500 distinct sets are coated with a reagent specific to a particular bioassay, allowing the capture and detection of specific analytes from a sample. In contrast, the present invention provides beads in a set that are first coated with a dockerin domain containing protein. Then each bead set is simply incubated with cohesin-antigen fusion proteins, which attach essentially irreversibly by non-covalent interaction. Thus, one dockerin- or cohesin-coated bead set can be used for multiple sets of cohesin- or dockerin-antigens, obviating the need to directly chemically couple new antigens to new beads. In a similar process the present invention provides beads in a set that are first coated with a cohesin domain containing protein. Then each bead set is simply incubated with dockerin-antigen fusion proteins, which attach essentially irreversibly by non-covalent interaction. Thus, one cohesin- or dockerin-coated bead set can be utilized for multiple sets of dockerin- or cohesin-antigens, obviating the need to directly chemically couple new antigens to new beads.

The present invention obviates the need for chemical cross-linking of antigens to beads and obviates the purification of the antigens from a recombinant expression system since the dockerin-beads can be used as an affinity matrix directly. The beads can be used as an affinity matrix directly resulting in beads ready for the assay. Also, this format presents the antigen in a configuration that does not obscure epitopes in contrast to chemical cross-linking techniques.

The present invention exploits the dockerin-cohesin pair interaction. The cohesin domains interact with small domains (e.g., 56 residues) called dockerins. These are Ca²⁺ containing structures with two-fold symmetry and they can bind to a cognate cohesin with various affinities. The interaction is non-covalent and is well defined (by structure analysis) for at least one dockerin-cohesin pair. Dockerins are designed to be domains linked to different domain (enzyme in nature), and cohesins are designed to function in linear arrays (either directly end-to-end, or joined by flexible PT-rich linkers of various sizes. It is known that a particular dockerin can have specificity for a particular cohesin (e.g., a dockerin-cohesin pair from one bacterial species may not be interchangeable with a dockerin-cohesin pair from a different species). This feature makes it is possible to ensure the specific and precise interaction of various fusion proteins with an engineered cohesin domains of various specificities.

Based on the crystal structure of the cohesin-dockerin complex it is apparent that one embodiment is an antigen-dockerin fusion proteins (i.e., antigen fused to the N-terminus of a dockerin). However, both from the structure and from the nature of cohesin domain organization within scaffolding, it is apparent that cohesins can be fused end-to-end, even without spacer sequences. Furthermore, it is apparent that well-described techniques are available to engineer miniaturized versions of the cohesin and dockerin domains.

Cellulosomes are multi-enzyme complexes that orchestrate the efficient degradation of cellulose and related plant cell wall polysaccharides. The complex is maintained by the high affinity protein-protein interaction between two complementary modules: the cohesin and the dockerin. Dockerin is a protein domain found in the cellulosome cellular structure. It is part of endoglucanase enzymes. The dockerin's binding partner is the cohesin domain. This interaction is essential to the construction of the cellulosome complex. The dockerin domain has two tandem repeats of a non-EF hand calcium binding motif characterized by a loop-helix structure. There are three types of Dockerin domains: I, II and III which bind to Cohesin Type I, Cohesin Type II and Cohesin Type III respectively.

A common feature of the cellulosomes is that they consist of a large number of catalytic components arranged around noncatalytic scaffolding proteins. These proteins fundamentally consist of repetitive noncatalytic domains of about 140 residues, termed cohesin domains, and a carbohydrate-binding module (CBM). For example, C. josui CipA N-terminus is composed of 3 CBMs followed by a hydrophilic domain and six cohesin domains; and C. thermocellum CipA contains a CBM between the second and third of nine repeated cohesin domains and a type II dockerin domain at its C terminus. The amino acid sequences of all the cohesin domains from these bacteria are in many cases highly homologous to each other, especially within the same species. Each cohesin domain is a subunit-binding domain that interacts with a docking domain, called dockerin, of each catalytic component. The dockerin domain contains two segments, also known as conserved duplicated regions (CDRs), each of which contains a Ca^{2 +}-binding loop and an alpha helix (namely, the calcium binding motif). An additional alpha helix intervenes between the two segments. The alpha helix in each duplicated sequence contains a conserved KR or KK dipeptide. The species-specific attachment of the dockerin module to the cohesin module is mediated via a high affinity Ca²⁺-dependent interaction. The cellulosome contains a CBM and a series of cohesin modules that anchor the cellulosomal enzymes to the multienzyme complex. The various cellulosomal enzymes contain inter alia a conserved dockerin module that binds to the cohesin counterpart. Biochemical and structural studies on the cohesins-dockerin interaction from have shown that the dockerins can bind to each of the cohesins on the scaffoldin with a strong affinity constant. Binding is can be reversible by addition of divalent ion chelators such as EDTA.

The present invention provides a cost-effective method and composition for connecting antigens to beads to obviate chemical cross-linking and purification of antigens from recombinant expression systems since the dockerin-beads can be used as an affinity matrix directly resulting in beads ready for the assay. Also, there is a need for an antigen configuration that does not obscure epitopes in contrast to chemical cross-linking techniques.

The solid substrate of methods and compositions of the present invention is, in another embodiment, a bead. In another embodiment, the solid substrate is a cell. In another embodiment, the solid substrate is an extracellular matrix. In another embodiment, the solid substrate is a fibrous matrix. In another embodiment, the solid substrate is a container. In another embodiment, the container is selected from the group consisting of a beaker, a flask, a cylinder, a test tube, a centrifugation tube, Petri dish, a culture dish, a multi-well plate or a chip. In another embodiment, the solid substrate is attached to or associated with an affinity column. Each possibility represents a separate embodiment of the present invention.

In another embodiment, an antibody-binding moiety is attached to a solid substrate of the present invention via fusion of the antibody-binding moiety to a truncated dockerin polypeptide. The truncated dockerin polypeptide is able to attach to a cohesin-containing protein bound to the solid substrate.

In another embodiment, a solid substrate of methods and compositions of the present invention comprises cellulose, and the protein bound to the solid substrate further comprises a carbohydrate-binding module (CBM). In another embodiment, the means of attachment of the protein to the solid substrate is via interaction between the CBM and the cellulose. Each possibility represents a separate embodiment of the present invention.

The molecule of interest of the methods and compositions of the present invention is any molecule that can be bound covalently, either directly or indirectly, to the truncated dockerin (e.g., truncated dockerin, substituted dockerin or modified dockerin) domain containing as disclosed herein. In various embodiments, the molecule of interest is any type of molecule which is desirable to purify or for which it is desirable to engineer an association with a solid substrate. In another embodiment the, the molecule of interest of the methods and compositions of the present invention is any molecule that can be bound covalently, either directly or indirectly, to the cohesin (e.g., truncated cohesin, substituted cohesin or modified cohesin) domain. In various embodiments, the molecule of interest is any type of molecule which is desirable to purify or for which it is desirable to engineer an association with a solid substrate.

In certain embodiments the molecule of interest is a peptide. In another embodiment, the molecule of interest is a protein. In another embodiment, the peptide is an enzyme. In another embodiment, the molecule is a peptide hormone. In another embodiment, the molecule is a recombinant peptide. In another embodiment, the molecule is a nucleic acid. In another embodiment, the molecule is a messenger. In another embodiment, the molecule is a drug. In another embodiment, the molecule is a cell receptor. In another embodiment, the molecule is a cell. In another embodiment, the molecule of interest is any other type of molecule for which it is desirable to purify or to engineer an association with a solid substrate.

As provided herein, a variety of proteins can be successfully purified with high-efficiency under gentle conditions following fusion to dockerin domains of the present invention. Methods for identification of dockerin domains are well known in the art. The dockerin-cohesin pair utilized in methods and compositions of the present invention are, in another embodiment, from the same species. Dockerins have been shown to bind to each of the cohesins on the scaffolding; thus, any cohesin from a given species is expected to bind any dockerin from that species. Cohesin-dockerin interactions are not species-specific; however, in some cases the interaction may be species-specific.

In another embodiment, the Kₐ of the dockerin domain (or substituted or modified dockerin domain) with the wild-type cohesin, in the presence of EDTA is low enough to act as a reversible affinity tag. In another embodiment, the Kₐ of this combination is under 10⁷M⁻¹. In another embodiment, the Kₐ of this combination is under 3×10⁶ M⁻¹. In another embodiment, the Kₐ of this combination is under 10⁶M⁻¹. In another embodiment, the Kₐ of this combination is under 3×10⁵ M⁻¹. In another embodiment, the Kₐ of this combination is under 10⁵ M'11. In another embodiment, the Kₐ of this combination is under 3×10⁴ M⁻¹. In another embodiment, the Kₐ of this combination is under 10⁴ M⁻¹. In another embodiment, the Kₐ of this combination is under 5×10³ M⁻¹. In another embodiment, the Kₐ of this combination is under 2×10⁵ M⁻³. In another embodiment, the Kₐ of this combination is under 10³ M⁻¹. In another embodiment, the Kₐ of this combination is under 5×10² M⁻¹. In another embodiment, the Kₐ of this combination is under 2×10² M⁻¹. In another embodiment, the Kₐ of this combination is under 10² M⁻¹. In another embodiment, the Kₐ of this combination is under 5×10¹ M⁻¹. In another embodiment, the Kₐ of this combination is under 2×10¹ M⁻¹. In another embodiment, the Kₐ of this combination is under 10¹ M⁻¹. Each possibility represents a separate embodiment of the present invention.

The cohesin domain of methods and compositions of the present invention is, in another embodiment, a Type-I cohesin domain. In another embodiment, the cohesin domain is a Type-II cohesin domain. In another embodiment, the cohesin domain is any other type of cohesin domain known in the art. Each possibility represents a separate embodiment of the present invention.

The present invention uses cohesin-dockerin from different species or sequences to make multivalent compositions. The invention includes the use of all cohesin-dockerin sequences from diverse cellulose degrading microbes, but describes the application of specific cohesin and dockerin and linker sequences from the microbe Clostridium thermocellum. The cohesin dockerin pairing exists in diverse cellulose degrading species. While they have sequence similarities, they can have specificities that do not cross between species. This affords an opportunity to build novel constructs with different specificities in a spatially and numerically controlled manner. By extension multiple cohesin-dockerin specificities can be used to make bivalent substrates with higher order antigen specificities. Cellulose degrading bacteria and similar organisms also use cellulose-binding domains to organize the degradation machinery.

The invention encompasses the use of entities to assemble spatially and numerically ordered complexes and multi subunit receptors.

In another embodiment, the cohesin domain is from a species selected from the group consisting of Clostridium thermocellum, C. cellulolyticum, and C. cellulovorans. In another embodiment, the cohesin domain is from a species selected from the group consisting of Clostridium thermocellum, C. papyrosolvens, and Clostridium cellobioparum. In another embodiment, the cohesin domain is from a species selected from the group consisting of Clostridium thermocellum, C. cellulolyticum, C. cellulovorans, C. papyrosolvens, C. josui, Acetivibrio cellulolyticus, Bacteroides cellulosolvens, R. flavefaciens, Ruminococcus albus, and Clostridium cellobioparum.

In another embodiment, the cohesin domain is a cohesin domain from a protein selected from CipA (or scaffoldin) of C. thermocellum, CipC of C. cellulolyticum, CbpA of C. cellulovorans, and CipA of C. josui. In another embodiment, the cohesin domain of methods and compositions of the present invention is from an Archaeoglobus fulgidus protein. In another embodiment, a cellulosomal cohesin domain is utilized in methods and compositions of the present invention. In another embodiment, a Type I cohesin domain from a cellulosomal protein is used. In another embodiment, a non-cellulosomal cohesin domain is utilized in methods and compositions of the present invention.

In one non-limiting example, the substrate of the present invention is engineered to have one or more modular cohesin-dockerin protein domains for making specific and defined protein complexes. In some embodiments the cohesin or dockerin protein domains may be from different species to allow for specific pairing of species-specific cohesin-dockerin proteins.

The substrates are alternately termed nanospheres, nanoparticles, microspheres, microparticles, nanobeads, microbeads, beads, polystyrene beads, latex particles, latex beads, fluorescent beads, fluorescent particles, colored particles and colored beads. The substrates serve as vehicles for molecular reactions. Illustrative microspheres and methods of manufacturing same are, for example, found in U.S. Pat. No. 7,141,431, the contents of which are incorporated by reference.

The substrate will be used to carry a separate molecule, e.g., a bead, a peptide, protein, lipid, carbohydrate, nucleic acid (oligonucleotide, aptamer, vector with or without base or backbone modifications) or combinations thereof by binding that separate molecule to the complementary half of the cohesin:dockerin pair. For example, either the dockerin or cohesin may be made into a fusion protein or chemically bound to an antigen, a peptide, a protein, a toxin, a cytokine, an enzyme, a structural protein, an extracellular matrix protein, another antibody, a cell or fragments thereof

The substrate may have one or more cohesin, dockerin or both cohesin and dockerin domains that allow the formation of a complex with one or more complementary cohesin/dockerin-molecules and a binding domain. The binding domain can be used to conjugate a protein, antibody, nucleic acid, dye, metal, label, active agent or any other composition. For example, the binding domain may be an antibody used to bind an antigen. In another embodiment the binding domain may be used to purify an antigen that binds to the antibody connected to the cohesin/dockerin-molecules and substrate.

The biological sample to be tested using the instant invention, for example, includes plasma, serum, tears, mucus, saliva, urine, pleural fluid, spinal fluid, gastric fluid, sweat, semen, vaginal secretion, fluid from ulcers and/or other surface eruptions, blisters, abscesses, and/or extracts of tissues, such as biopsies of normal, malignant, and/or suspect tissues.

The analytes of interest for these bioassays include, for example, antigens, antibodies, autoantibodies, peptides, proteins, nucleic acid sequences, and/or enzymes. The antigenic analytes, for example, includes bacterial, viral, fungal, mycoplasmal, ridkettsial, chlamydial, and/or protozoal antigens.

Examples of antigens include tumor proteins, e.g., mutated oncogenes; viral proteins associated with tumors; and tumor mucins and glycolipids. The antigens may be viral proteins associated with tumors would be those from the classes of viruses noted above. Certain antigens may be characteristic of tumors (one subset being proteins not usually expressed by a tumor precursor cell), or may be a protein which is normally expressed in a tumor precursor cell, but having a mutation characteristic of a tumor. Other antigens include mutant variant(s) of the normal protein having an altered activity or subcellular distribution, e.g., mutations of genes giving rise to tumor antigens. Specific non-limiting examples of tumor antigens include: CEA, prostate specific antigen (PSA), HER-2/neu, BAGE, GAGE, MAGE 1-4, 6 and 12, MUC (Mucin) (e.g., MUC-1, MUC-2, etc.), GM2 and GD2 gangliosides, ras, myc, tyrosinase, MART (melanoma antigen), Pmel 17(gp100), GnT-V intron V sequence (N-acetylglucoaminyltransferase V intron V sequence), Prostate Ca psm, PRAME (melanoma antigen), beta-catenin, MUM-1-B (melanoma ubiquitous mutated gene product), GAGE (melanoma antigen) 1, BAGE (melanoma antigen) 2-10, c-ERB2 (Her2/neu), EBNA (Epstein-Barr Virus nuclear antigen) 1-6, gp75, human papilloma virus (HPV) E6 and E7, p53, lung resistance protein (LRP), Bc1-2, and Ki-67. In addition, the immunogenic molecule can be an autoantigen involved in the initiation and/or propagation of an autoimmune disease, the pathology of which is largely due to the activity of antibodies specific for a molecule expressed by the relevant target organ, tissue, or cells, e.g., SLE or MG. In such diseases, it can be desirable to direct an ongoing antibody-mediated (i.e., a Th2-type) immune response to the relevant autoantigen towards a cellular (i.e., a Th1-type) immune response. Alternatively, it can be desirable to prevent onset of or decrease the level of a Th2 response to the autoantigen in a subject not having, but who is suspected of being susceptible to, the relevant autoimmune disease by prophylactically inducing a Th1 response to the appropriate autoantigen. Autoantigens of interest include, without limitation: (a) with respect to SLE, the Smith protein, RNP ribonucleoprotein, and the SS-A and SS-B proteins; and (b) with respect to MG, the acetylcholine receptor. Examples of other miscellaneous antigens involved in one or more types of autoimmune response include, e.g., endogenous hormones such as luteinizing hormone, follicular stimulating hormone, testosterone, growth hormone, prolactin, and other hormones. Antigens involved in autoimmune diseases, allergy, and graft rejection can be used in the compositions and methods of the invention. For example, an antigen involved in any one or more of the following autoimmune diseases or disorders can be used in the present invention: diabetes, diabetes mellitus, arthritis (including rheumatoid arthritis, juvenile rheumatoid arthritis, osteoarthritis, psoriatic arthritis), multiple sclerosis, myasthenia gravis, systemic lupus erythematosis, autoimmune thyroiditis, dermatitis (including atopic dermatitis and eczematous dermatitis), psoriasis, Sjogren's Syndrome, including keratoconjunctivitis sicca secondary to Sjogren's Syndrome, alopecia areata, allergic responses due to arthropod bite reactions, Crohn's disease, aphthous ulcer, iritis, conjunctivitis, keratoconjunctivitis, ulcerative colitis, asthma, allergic asthma, cutaneous lupus erythematosus, scleroderma, vaginitis, proctitis, drug eruptions, leprosy reversal reactions, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Crohn's disease, Graves opthalmopathy, sarcoidosis, primary biliary cirrhosis, uveitis posterior, and interstitial lung fibrosis. Examples of antigens involved in autoimmune disease include glutamic acid decarboxylase 65 (GAD 65), native DNA, myelin basic protein, myelin proteolipid protein, acetylcholine receptor components, thyroglobulin, and the thyroid stimulating hormone (TSH) receptor. Examples of antigens involved in allergy include pollen antigens such as Japanese cedar pollen antigens, ragweed pollen antigens, rye grass pollen antigens, animal derived antigens such as dust mite antigens and feline antigens, histocompatiblity antigens, and penicillin and other therapeutic drugs. Examples of antigens involved in graft rejection include antigenic components of the graft to be transplanted into the graft recipient such as heart, lung, liver, pancreas, kidney, and neural graft components. The antigen may be an altered peptide ligand useful in treating an autoimmune disease.

In one example, the present invention can be used in any system for multiplex detection of agents. For example, color-coded beads, pre-coated with analyte-specific capture dockerin or cohesin bound to the beads for the molecule of interest, are added. Either the cohesin or dockerin is bound to the bead, with the analyte bound to the other half of the cohesin-dockerin pair. Multiple analytes can be simultaneously detected in the same sample by binding to the dockerin or cohesin, e.g., as a fusion protein. The analyte (e.g., antigens, antibodies, autoantibodies, peptides, proteins, nucleic acids, enzymes, or small molecules) can be bound, loaded or linked (covalently or non-covalently) to the dockerin or cohesin matching pair on the beads.

Next, analyte-specific antibodies are added to the beads to capture the analyte of interest. In one embodiment, biotinylated detection antibodies specific to the analyte of interest are added and form an antibody-antigen sandwich.

In the case of a biotinylated agent, a detectable agent (e.g., Phycoerythrin (PE)) conjugated to Streptavidin is added. In another embodiment, the dockerin-cohesin pair can substitute for the biotin-streptavidin, wherein one portion of the binding pair is bound to a detectable label (fluorescent, magnetic, radioactive, electron-dense, enzymatic, and the like).

The beads are read using, e.g., a dual-laser flow-based detection instrument, such as the Luminex 200™ or Bio-Rad® Bio-Plex® analyzer. One laser classifies the bead and determines the analyte that is detected. The second laser determines the magnitude of the signal derived from the detectable label. The signal from the detectable label is generally proportional to the amount of bound analyte. For example, the analyte can be a lymphokine such as, e.g., IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32, IL-33, IL-34, IL-35, and/or, IL-36, and variants thereof Examples of cytokines and other agents that can be used as the analyte include, e.g., granulocyte-colony stimulating factor, macrophage-colony stimulating factor, granulocyte-macrophage colony stimulating factor, leukemia inhibitory factor, erythropoietin, granulocyte macrophage colony stimulating factor, oncostatin M, leukemia inhibitory factor, IFN-alpha, -beta, or -gamma, lymphotoxin -alpha or -beta, CD40 ligand, Fas ligand, CD27 ligand, CD30 ligand, 4-1BBL, TGF-alpha or -beta, IL-1 RA, macrophage inhibitory factor, and/or a mixture thereof The beads can be color-coded or comprise a detectable label or bar code that identifies the bead. Different beads can be coated with either dockerin or cohesin and a specific analyte, bound to the other half of the dockerin-cohesin pair, is added to the coated bead to make the bead specific to a single analyte.

Example of sequence encoding C1.C2.C3.Cn is taken from the public sequence >gi|50656899|gb|AAT79550.1| of cellulosomal anchoring scaffoldin B precursor (Bacteroides cellulosolvens). Below in bold showing the leader secretion sequence and italics and bold-underlined highlighting various cohesin domains. Italics-underlined regions are linkers spacing some of the cohesin domains. The skilled artisan will easily be able to make one or more of these domains as individual domains, as concatamers, as multimers and to provide for isolation of the same, e.g., by adding recognition domains such as His-tags and the like. Nucleic acid sequences that encode the domains (cohesin or dockerin) can easily be synthesized and customized to the specific expression system used, e.g., bacterial, fungal, helminthic or mammalian.

(SEQ ID NO: 1)
MQSPRLKRKILSVILAVCYIISSFSIQFAATPQVNIIIGSAQGIPGSTVKVPINLQNVPEIGIN
NCDFTIKFDSDILDFNSVEAGDIVPLPVASFSSNNSKDIIKFLFSDATQGNMPINENGLFAVISF
KIKDNAQKGISNIKVSSYGSFSGMSGKEMQSLSPTFFSGSIDVSDVSTSKLDVKVGNVEGIA
GTEVNVPITFENVPDNGINNCNFTLSYDSNALEFLTTEAGNIIPLAIADYSSYRSMEG
KIKFLFSDSSQGTRSIKNDGVFANIKFKIKGN [symbol] [symbol]
SSVSPTTSVHPTPTSVPPTPTKSSPGNKMKIQIGDVKANQGDTVIVPITF
NEVPVMGVNNCNFTLAYDKNIMEFISADAGDIVTLPMANYSYNMPSDGLVKFLYNDQAQGA
MSIKEDGTFANVKFKIKQSAAFGKYSVGIKAIGSISALSNSKLIPIESIFKDGSIT
VTNKPIVNIEIGKVKVKAGDKIKVPVEIKDIPSIGINNCNFTLKYNSNVLKYVSNEAG
TIVPAPLANLSINKPDEGIIKLLFSDASQGGMPIKDNGIFVNLEFQAVNDANIGVYGL
ELDTIGAFSGISSAKMTSIEPQFN [symbol] QTPTNTISVTPTNNSTPT
NNSTPKPNPLYNLNVNIGEISGEAGGVIEVPIEFKNVPDFGINNCDFSVKYDKSIFEYVTYEAG
SIVKDSIVNLACMENSGIINLLFNDATQSSSPIKNNGVFAKLKFKINSNAASGTYQINAEGYGKF
SGNLNGKLTSINPIFENGIINIGNVTVKPTSTPADSSTITPTATPTATPTIKGTPTVTPIYWMNV
LIGNMNAAIGEEVVVPIEFKNVPPFGINNCDFKLVYDSNALELKKVEAGDIVPEPLA
NLSSNKSEGKIQFLFNDASQGSMQIENGGVFAKITFKVKSTAASGIYNIRKDSVGSF
SGLIDNKMTSIGPKFTDGSIVVGTVTPTATATPSAIVTTITPTATTKPIATPTIKGTPTATPMY
WMNVVIGKMNAEVGGEVVVPIEFNNVPSFGINNCDFKLVYDATALELKNVEAGDIIKTPLAN
FSNNKSEEGKISFLFNDASQGSMQIENGGVFAKITFKVKSTTATGVYDLRKDLVGSFSGLKDN
KMTSIGAEFTNGSITVAATAPTVTPTVNATPSAATPTVTPTATATPSVTIPTVTPTATATPSVTIPT
VTPTATATPSAATPTVTPTATATPSVTIPTVTPTVTATPSDTIPTVTPTATATPSAIVTTITPTATAK
PIATPTIKGTPTATPMYWMNVVIGKMNAEVGGEVVVPIEFKNVPSFGINNCDFKLVY
DATALELKNVEAGDIIKTPLANFSNNKSEEGKISFLFNDASQGSMQIENGGVSAKIT
FKVKSTTAIGVYDIRKDLIGSFSGLKDSKMTSIGAEFTNGSITVATTAPTVTPTATATPS
VTIPTVTPTATATPGTATPGTATPTATATPGAATPTETATPSVMIPTVTPTATATPTATATPTVK
GTPTIKPVYKMNVVIGRVNVVAGEEVVVPVEFKNIPAIGVNNCNFVLEYDANVLEVKKVDAG
EIVPDALINFGSNNSDEGKVYFLFNDALQGRMQIANDGIFANITFKVKSSAAAGIYNIRKDSVG
AFSGLVDKLVPISAEFTDGSISVESAKSTPTATATGTNVTPTVAATVTPTATPASTTPTAT
PTATSTVKGTPTATPLYSMNVIIGKVNAEASGEVVVPVEFKDVPSIGINNCNFILEY
DASALELDSAEAGEIVPVPLGNFSSNNKDEGKIYFLFSDGTQGRMQIVNDGIFAKIK
FKVKSTASDGTYYIRKDSVGAFSGLIEKKIIKIGAEFTDGSITVRSLTPTPTVTPNVAS
PTPTKVVAEPTSNQPAGPGPITGTIPTATTTATATPTKASVATATPTATPIVVVEPTIVRP
GYNKDADLAVFISSDKSRYEESSIITYSIEYKNIGKVNATNVKIAAQIPKFTKVYDAAKG
AVKGSEIVWMIGNLAVGESYTKEYKVKVDSLTKSEEYTDNTVTISSDQTVDIPENITTG
NDDKSTIRVMLYSNRFTPGSHSSYILGYKDKTFKPKQNVTRAEVAAMFARIMGLTVKD
GAKSSYKDVSNKHWALKYIEAVTKSGIFKGYKDSTFHPNAPITRAELSTVIFNYLHLNNI
APSKVHFTDINKHWAKNYIEEIYRFKLIQGYSDGSFKPNNNITRAEVVTMINRMLYRGP
LKVKVGSFPDVSPKYWAYGDIEEASRNHKYTRDEKDGSEILIE

The cohesin domains (C) interact with small domains (e.g., 56 residues) called dockerins

(D). These are Ca⁺⁺ containing structures with two-fold symmetry and they can bind to a cognate cohesin with various affinities (e.g., 6E6 M, 2E7M). Affinities between dockerin and multiple cohesins (as found on scaffoldins) can be much higher (e.g., >E9 M). The interaction is non-covalent and is well defined (by structure analysis) for at least one C-D pair. Dockerins are designed to be domains linked to different domain (enzyme in nature), and cohesions are designed to function in linear arrays (either directly end-to-end, or joined by flexible PT-rich linkers of various sizes (e.g., 12, 17, 25, 28, 36). It is known that a particular dockerin can have specificity for a particular cohesin (e.g., a C-D pair from one bacterial species may not be interchangeable with a C-D pair from a different species). This feature makes it is possible to ensure the specific and precise interaction of various D-antigen fusion proteins with an engineered mAb containing cohesin domains of various specificities.

Dockerin:

(SEQ ID NO: 2)
GDVNDDGKVNSTDLTLLKRYVLKAVSTLPSSKAEKNADVNRDGRVD
VTILSRYLIRVIEKLPI

In practice, this invention includes adapting C-D pairs known from the literature, newly gleamed from nature, or developed with new specificities using phage display technology. The latter technology can also be used to enhance (‘mature’) the affinity of a C-D interaction, should this be desired. Also, engineering cysteine residues at opposing faces of the C-D interaction (based on modeling from the published C-D structures) could be used to make a covalent bond between C-D to strengthen the interaction. Furthermore, the dimeric nature of the mAb (and therefore the linked C-domains) can be used to advantage for affinity enhancement purposes. In this embodiment, e.g., the D-antigen fusion protein is engineered either with a second identical dockerin domain (D-antigen-D, or D-D-antigen), or with a homodimerization domain. This configuration, provided the linkers between domains were not constraining, will result in the preferred simultaneous binding of both D domains to the same mAb, with greatly enhanced stability compared to the single interaction.

Based on the crystal structure of the cohesin-dockerin complex (e.g., see PNAS 2003,13809-13814, Cellulosome assembly revealed by the crystal structure of the cohesin-dockerin complex. Ana L. Carvalho, Fernando M. V. Dias, José A. M. Prates, Tibor Nagy, Harry J. Gilbert, Gideon J. Davies, Luis M. A. Ferreira, Maria J. Romão and Carlos M. G. A. Fonte), it is apparent that one embodiment is an antigen-dockerin fusion proteins (i.e., antigen fused to the N-terminus of a dockerin). However, both from the structure and from the nature of cohesin domain organization within scaffoldins, it is apparent that cohesions can be fused end-to-end, even without spacer sequences. Furthermore, it is apparent that well-described techniques are available to engineer miniaturized versions of the cohesin and dockerin domains (see for example, Proc. Natl. Acad. Sci. USA Vol. 94, pp. 10080-10085, September 1997. Structural mimicry of a native protein by a minimized binding domain. Melissa A. Starovasnik, Andrew C. Braisted, And James A. Wells).

It is recognized herein that the linker sequences have a propensity for O-linked glycosylation resulting from ST richness. Also, both the C and D domains can have potential N-linked sites. These features can be advantageous in enhancing the solubility of the mammalian cell-expressed engineered mAb through decoration with carbohydrates. Of course, the consequences of glycosylation of the C domains needs to be check by function (binding to the cognate D), and if needed rectified by site directed mutagenesis. An attractive feature of this invention is that D-A can be expressed in whatever system is known to be best. The dockerin domain is below.

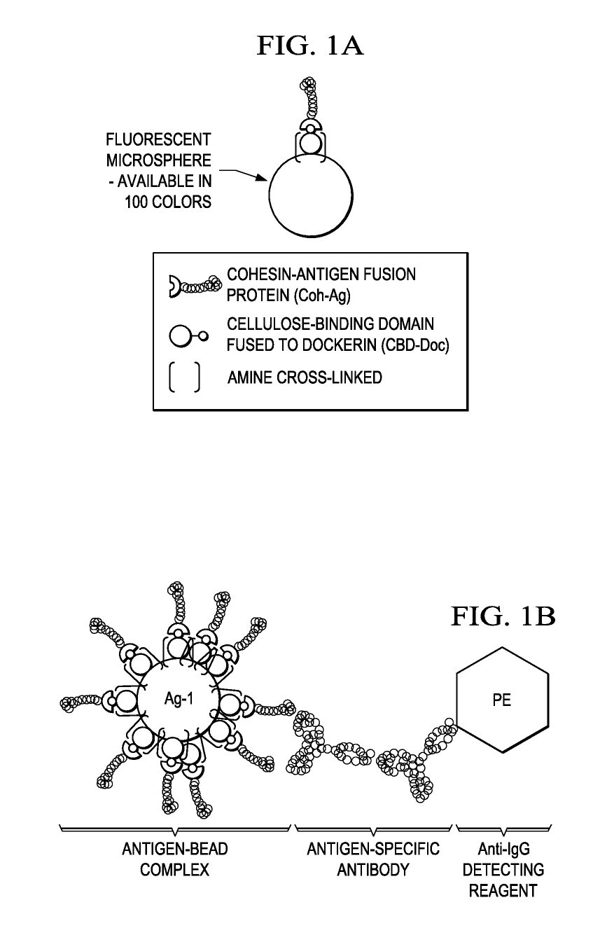

The present inventors have developed a flexible, sensitive, accurate, high throughput, sample sparing, multiplexed bead-based assay for the simultaneous measurement of antibodies against multiple antigens. This is represented schematically in FIGS. 1A and 1B.

FIGS. 1A and 1B show that cohesin and dockerin are protein modules from cellulose-degrading bacteria that bind non-covalently with very high affinity (K_{D}˜30 μM). In FIG. 1A a set of up to 100 different fluorescent microspheres (e.g., Luminex® beads) are cross-linked chemically to a cellulose-binding domain fused to dockerin (CBD-Doc). Each cohesin-antigen fusion protein is mixed to CBD-Doc beads of a particular color (e.g., bead region 21). The beads are washed and pooled with CBD-Doc beads similarly coated with other desired cohesin-antigen fusion proteins. In FIG. 1B the bead-antigen sets are incubated with a dilution series of each serum sample, washed, incubated with e.g., anti-human IgG-PE conjugate, washed again, then read in a flow cytometry instrument which measures fluorescence intensity associated with each bead color.

A sample protocol for confirmation of antibody coupling is presented herein below. Microspheres should be protected from prolonged exposure to light throughout this procedure.
(i). Select the appropriate antibody-coupled microsphere sets.
(ii). Resuspend the microspheres by vortex and sonication for approximately 20 seconds.
(iii). Prepare a Working Microsphere Mixture by diluting the coupled microsphere stocks to a final concentration of 100 microspheres of each set/μL in PBS-1% BSA. (Note: 50 μL of Working Microsphere Mixture is required for each reaction. Either PBS-1% BSA or PBS-BN (PBS, 1% BSA, 0.05% Azide, pH 7.4) may be used as Assay Buffer. Not using Azide.

(iv). Prepare two-fold serial dilutions of phycoerythrin-labeled anti-species IgG detection antibody from 4 to 0.0625 μg/mL in PBS-1% BSA. (Note: 50 μL of diluted detection antibody is required for each reaction.)
(v). Pre-wet a 1.2 μm Millipore filter plate with 100 μL/well of PBS-1% BSA and aspirate by vacuum manifold.
(vi). Aliquot 50 μL of the Working Microsphere Mixture into the appropriate wells of the filter plate.
(vii). Add 50 μL of the diluted detection antibody into the appropriate wells of the filter plate.
(viii). Mix the reactions gently by pipetting up and down several times with a multi-channel pipettor.
(ix). Cover the filter plate and incubate for 30 minutes at room temperature on a plate shaker. (let go 1.5 hours)
(x). Aspirate the supernatant by vacuum manifold.
(xi). Wash each well twice with 100 μL of PBS-1% BSA and aspirate by vacuum manifold.
(xii). Resuspend the microspheres in 100 μL of PBS-1% BSA by gently pipetting up and down five times with a multi-channel pipettor.

(xiii). Analyze 50-75 μL on the Luminex analyzer according to the system manual.

This multiplex assay format described hereinabove is very robust. Samples rerun independently give virtually identical results. Bead sets are monitored to ensure equivalent loading of each Cohesin-antigen fusion protein, thereby enabling direct comparison of titers against comparable antigens (e.g., HA-1 from PR8 vs. H1N1 swine flu). Data for cross-reactive antigens are equivalent if the related antigens are run separately, validating the use of the multiplexed format to derive this data.

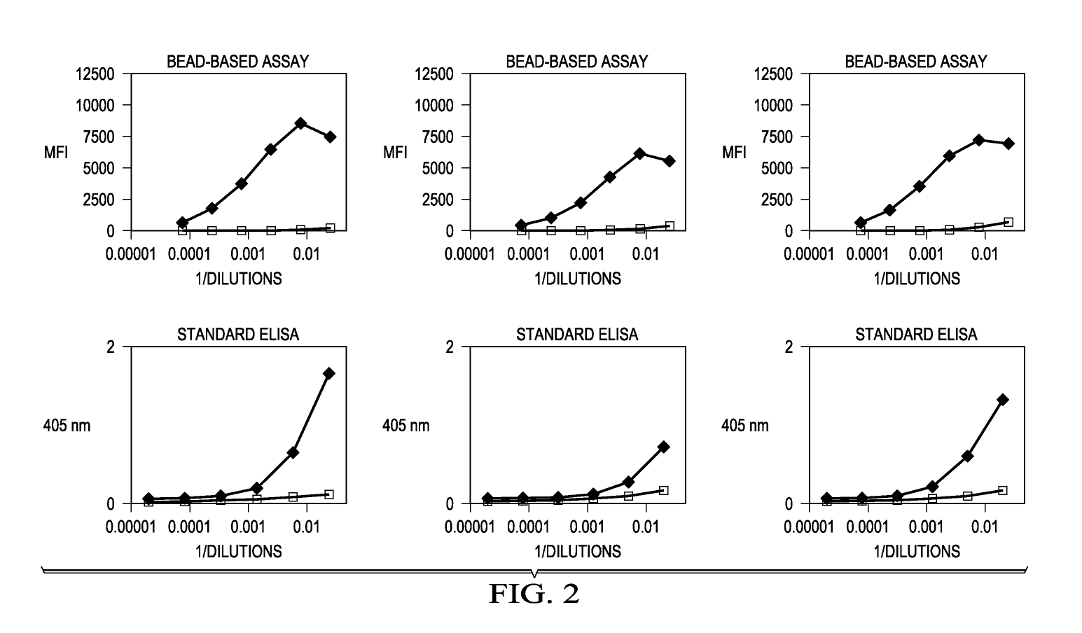

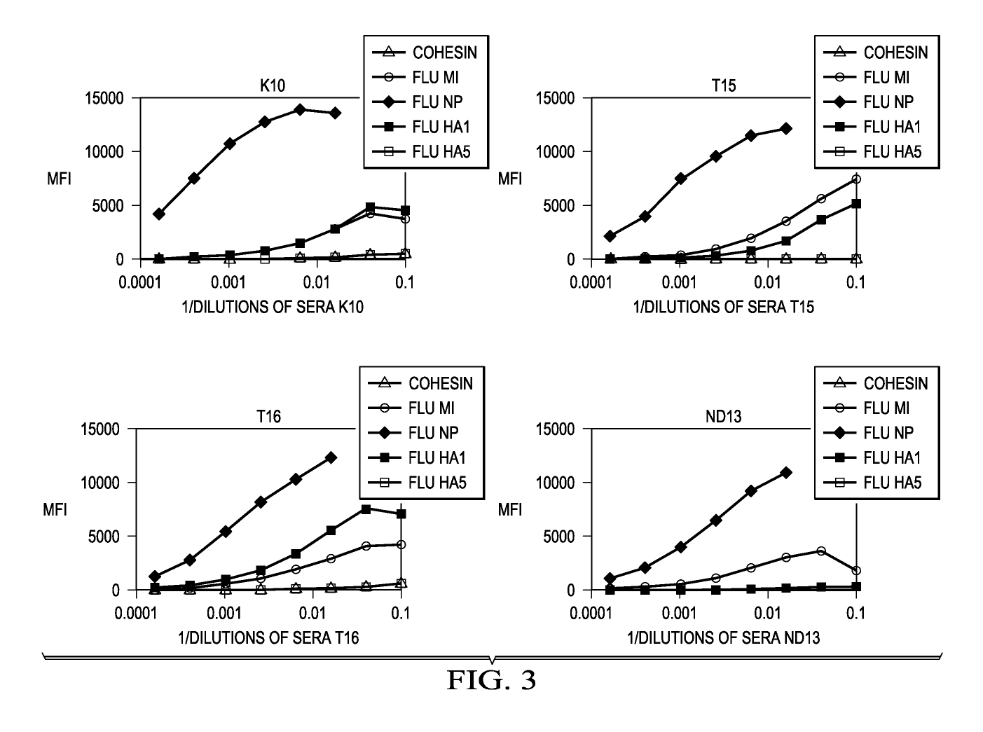

Also, there is no detectable exchange of Cohesin-antigens between bead sets during the assay. The Dockerin-Cohesin binding requires 8M guanidine to disrupt the interaction and survives in strong acid and base conditions. The assay described herein is at least 10-fold more sensitive than a standard solid phase ELISA using the same antigen. FIG. 2 shows the comparison of serum HIV Gag p24-specific antibody titers in patients acquired by bead assay or conventional ELISA using commercial reagents. FIG. 3 shows the influenza antigen-specific IgG levels in healthy donors tested by the bead assay of the present invention. Typically serum dilution series start at 1:10 and use 20 μl total serum. Because the antigen is coupled to beads via the Dockerin-Cohesin interaction, all antigen epitopes are available for binding antibody, uncompromised by non-specific plate binding or chemical cross-linking effects.

Recombinant human IgG-Cohesin fusion protein can be run as an internal standard. For e.g., to configure and execute antigen-specific assays for the Influenza antigens M1 (PR8), NP (PR8, H1N1 swine flu, H1N1 avian flu), M2e (PR8, H1N1 swine Flu), HA-1 (PR8, H1N1 avian flu, H1N1 swine flu, and seasonal flu strains). Pure proteins, and E. coli expression constructs or stably transfected CHO-S cell lines are already established along with validated purification protocols for all these antigens including HA-1 proteins from new seasonal flu strains.

In another example, isotype-specific antigen-specific assay can be done in the context of the multiplexed bead assay. The current standard assay configuration uses a pan anti-IgG Fc reagent (couples to PE for fluorescent read-out) to detect bound antibodies. The present invention provides the capacity to detect Ig subclass-specific antibodies by adapting commercially available isotype-specific detection reagents (e.g., anti-IgM-PE, anti-IgA-PE). To this end, the present inventors have made a panel of recombinant antibodies by systematically grafting H chain constant regions of each isotype onto the same variable region specificity (e.g., from an existing anti-human CD40 antibody construct). These reagents permit the assembly of a fully matched antibody-coated multiplexed bead set for the same antigen-specificity (e.g., beads coated with Cohesin-CD40 ectodomain fusion protein and then bound to saturation with the anti-CD40 antibody of each isotype. This permits sensitive and accurate appraisal and quality assurance of new isotype-specific detection reagents. It has been found that commercially available isotype-specific detection reagents are of mixed quality, so the set-up of the present invention allows strict quality control of these reagents for cross-reactivity.

The present invention enables quantifying antigen specific blood B cells based on the same set of Cohesin-antigen and CBD-Dockerin reagents that have been successfully deployed hereinabove for antigen-specific multiplexed bead assay. This also obviates the need for making new protein reagents.

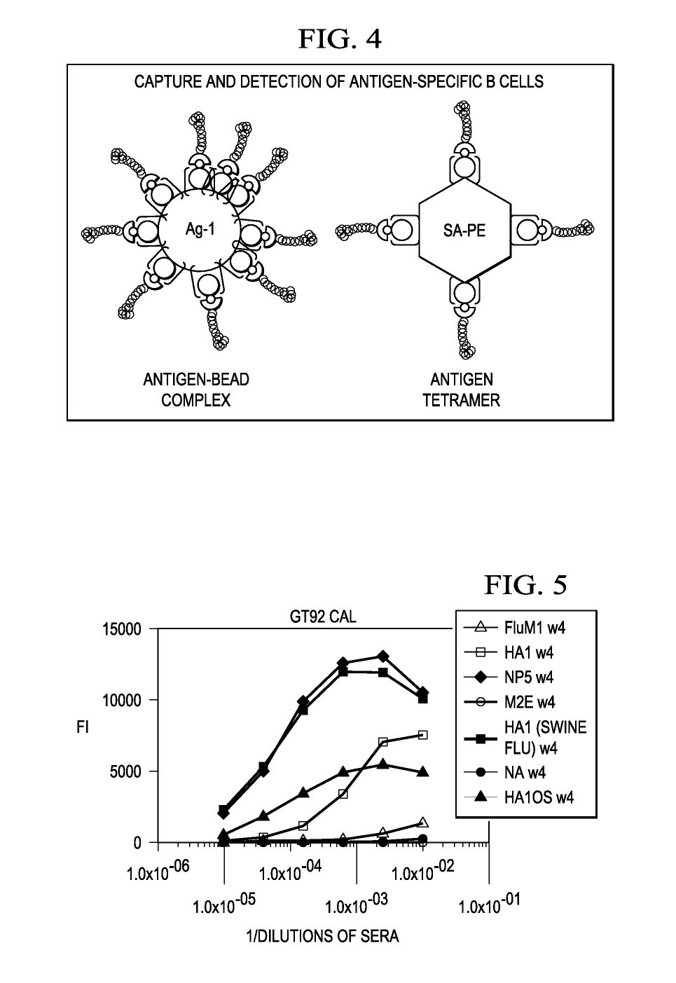

Reagents for capture and detection of antigen-specific B cells are shown in FIG. 4. Beads displaying a Cohesin-antigen fusion protein (Ag-1) can be used to enrich antigen-specific B cells from ex vivo samples, while antigen tetramers assembled via streptavidin-phycoerythrin (SA-PE) : biotin-CBD-Doc : Cohesin-antigen complex can be uses in flow cytometry to sort or quantify antigen-specific B cells. The antigen-bead complexes can also be assembled on biotin-coated Q dots of different, permitting the possible development of multiplexed flow analysis of several antigen-specific B cell categories. Furthermore, the combinatorial strategy of mixing tetramers for each specificity but with two different colors [Newell et al., Nature Methods 6, 497 - 499 (2009)] can be readily applied to our antigen tetramers to reduce background staining and increase the number of antigens that can be tested simultaneously.

FIG. 5 demonstrates the method of the present invention using tetramers made of

Cohesin-Dockerin, which can recognize receptors expressed on cell surface. The plot shown in FIG. 5 is a multiplex bead-based assay of antigen-specific serum antibodies from a Influenza-infected non-human primate. Three different HA antigens are represented. As well as Ml, M2 ectodomain, NP, and NA.

FIG. 5 demonstrates the capacity of the multiplex bead-based assay to simultaneously measure both antigen-specific titers (by limiting dilution, or the highest dilution to give a measurable response, but also cross-reactivity. For example, this sera contains a high titer of antibodies specific to HAl (swine flu) shown in green squares (this animal was infected with swine flu and had no detectable anti-flu antigen antibodies before the infection), but a lower titer on antibodies that are cross-reactive to a different HAl shown in red. The FI plateaus also reveals the complexity of the antibody response (a more complex response reflects a larger number of epitopes on the antigen were bound).

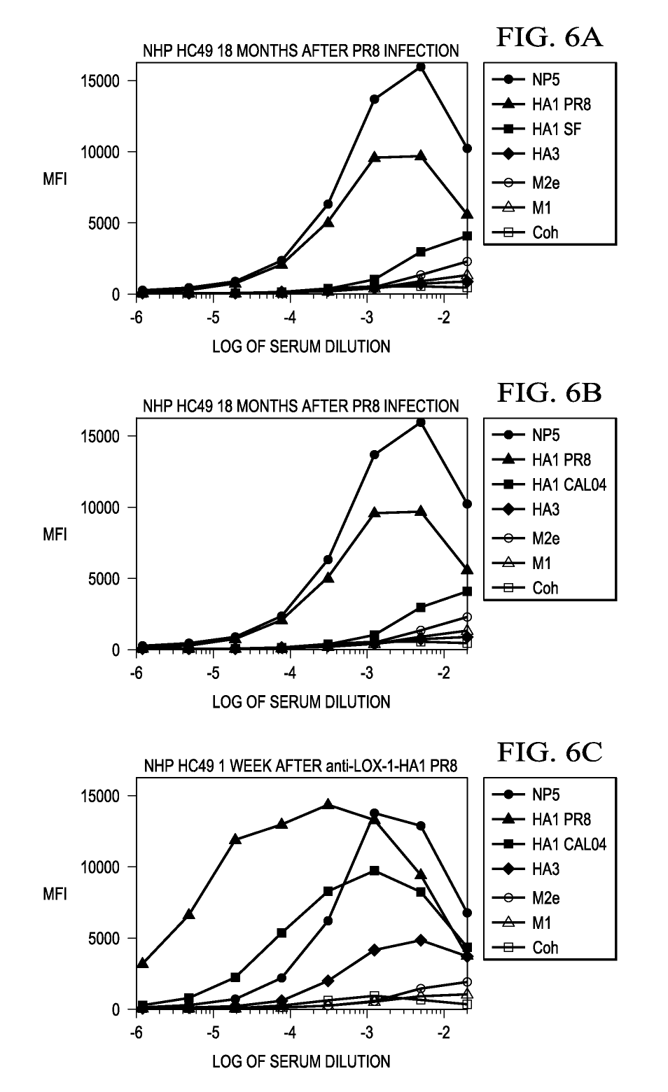

FIGS. 6A-6C provide an example of a multiplex bead antigen-specific IgG assay. In FIG. 6A a serum sample from an influenza-infected monkey was diluted and mixed with a bead set coated with cohesin fused to various Influenza proteins NP5, nuclear protein; HAl PR8,

HAl domain from hemagglutinin of the PR8 strain; HAl SF, HAl domain from hemagglutinin of the swine flu strain (CAL04); HA3, HAl domain from hemagglutinin a H3 Influenza strain; M2e, the ectodomain of the M2 protein from PR8; Ml, matrix protein 1; Coh, Cohesin without antigen. FIGS. 6B and 6C compares the serum from the monkey before (FIG. 6B) and after vaccination (FIG. 6C) with a vaccine bearing HAl from PR8.

C191 Ecoli-pET28[6xHis-CBD-Dockerin]

(SEQ ID NO: 3)
ATGGGCAGCAGCCATCATCATCATCATCACAGCAGCGGCCTGGTGCC
GCGCGGCAGCCATATGGCTAGTGGCAATGCAACACCGACCAAGGGAG
CAACACCAACAAATACAGCTACGCCGACAAAATCAGCTACGGCTACG
CCCACCAGGCCATCGGTACCGACAAACACACCGACAAACACACCGGC
AAATACACCGGTATCAGGCAATTTGAAGGTTGAATTCTACAACAGCA
ATCCTTCAGATACTACTAACTCAATCAATCCTCAGTTCAAGGTTACT
AATACCGGAAGCAGTGCAATTGATTTGTCCAAACTCACATTGAGATA
TTATTATACAGTAGACGGACAGAAAGATCAGACCTTCTGGTGTGACC
ATGCTGCAATAATCGGCAGTAACGGCAGCTACAACGGAATTACTTCA
AATGTAAAAGGAACATTTGTAAAAATGAGTTCCTCAACAAATAACGC
AGACACCTACCTTGAAATAAGCTTTACAGGCGGAACTCTTGAACCGG
GTGCACATGTTCAGATACAAGGTAGATTTGCAAAGAATGACTGGAGT
AACTATACACAGTCAAATGACTACTCATTCAAGTCTGCTTCACAGTT
TGTTGAATGGGATCAGGTAACAGCATACTTGAACGGTGTTCTTGTAT
GGGGTAAAGAACCCGGTGGCAGTGTAGTACCATCAACACAGCCTGTA
ACAACACCACCTGCAACAACAAAACCACCTGCAACAACAAAACCACC
TGCAACAACAATACCGCCGTCAGATGATCCGAATGCAGCTAGCAATT
CTCCTCAAAATGAAGTACTGTACGGAGATGTGAATGATGACGGAAAA
GTAAACTCCACTGACTTGACTTTGTTAAAAAGATATGTTCTTAAAGC
CGTCTCAACTCTCCCTTCTTCCAAAGCTGAAAAGAACGCAGATGTAA
ATCGTGACGGAAGAGTTAATTCCAGTGATGTCACAATACTTTCAAGA
TATTTGATAAGGGTAATCGAGAAATTACCAATATAA
(SEQ ID NO: 4)
MGSSHHHHHHSSGLVPRGSHMASGNATPTKGATPTNTATPTKSATAT
PTRPSVPTNTPTNTPANTPVSGNLKVEFYNSNPSDTTNSINPQFKVT
NTGSSAIDLSKLTLRYYYTVDGQKDQTFWCDHAAIIGSNGSYNGITS
NVKGTFVKMSSSTNNADTYLEISFTGGTLEPGAHVQIQGRFAKNDWS
NYTQSNDYSFKSASQFVEWDQVTAYLNGVLVWGKEPGGSVVPSTQPV
TTPPATTKPPATTKPPATTIPPSDDPNAASNSPQNEVLYGDVNDDGK
VNSTDLTLLKRYVLKAVSTLPSSKAEKNADVNRDGRVNSSDVTILSR
YLIRVIEKLPI

SEQ ID NO: 3 represents a cellulose binding domain—dockerin domain fusion protein that is efficiently expressed in E. coli [using the pET28 vector system] as an abundant and soluble intracellular protein. Underlined region is GENE ID: 4809951 Cthe_{—}3077 1 cellulosome anchoring protein, cohesin region [Clostridium thermocellum ATCC 27405] residues 322-561; Residues indicated in bold are sp|P0C2S4.1|GUND_CLOTM RecName: Full=Endoglucanase D residues 551-625.

For production of this protein, pET28[6xHis-CBD-Dockerin] plasmid in a suitable E. coli strain is grown in L broth to mid-log phase, induced with IPTG for ˜3 hours, then harvested by centrifugation. Cells are broken e.g., by sonication, in 50 mM Tris.HCl pH 7.5 buffer with 1 mM EDTA and a cocktail of standard protease inhibitors, then clarified by centrifugation (SS34 rotor, 14,500 r.p.m. 20 min). The supernatant is passed through Q sepharose, adjusted to 50 mM NaH₂PO₄, pH 8.0; 300 mM NaCl; 10 mM imidazole, and then loaded onto a Ni⁺⁺ charged metal chelating column, washed with 50 mM NaH₂PO₄, pH 8.0; 300 mM NaCl; 20 mM imidazole buffer containing 0.5% ASB14 to lower LPS levels, then eluted with gradient to 50 mM NaH²PO⁴, pH 8.0; 300 mM NaCl; 250 mM imidazole. Fractions containing eluted protein are pooled and buffer exchanged into 10 mM Borate pH 7.4. This protein retains both cohesin binding and (likely) cellulose binding activity.

The present invention can also be modified to multiplexed protein array format. For example, using a modification of a method described in Versatile protein microarray based on carbohydrate-binding modules. Proteomics 2005, 5, 1806-1814. Here the cohesin-antigen proteins can be simply spotted on a surface pre-coated with a dockerin domain-containing protein, which is chemically cross-linked to the surface, or if CBD.Doc, attached by non-covalent means to a cellulose coated surface.

Table 1 presents a list of examples of cohesin-antigen fusion proteins that were successfully expressed either in E. coli, or in mammalian cell systems by the present inventors. Since the cohesin portion is robust and can be functionally expressed as a secreted product in mammalian cells, or as a soluble, or refolded, product from E. coli this brings versatility since the best system for abundantly expressed and properly folded antigen fusion partner can be selected. The sequences corresponding to the cohesion-antigen fusion proteins are also presented herein below.

TABLE 1
Cohesin-Antigen fusion proteins expressed either in E. coli or in mammalian
cell systems.

| | | NUMBER/ |
|---|---|---|
| ANTIGEN | CONSTRUCT NAME | SEQ ID NO: |
| Autoantigen | Ecoli-pET28[Cohesin-hGadB-6xHis] | C914/5 |
| Bacterial | Ecoli-pET28[6xHis-Cohesin-Flgn-1-Flgn-2] | C1036/6 |
| Bacterial toxin | Ecoli-pET28[Cohesin-P. aeruginosaPE38] | C340/7 |
| Cancer | Ecoli-pET28[6xHis-CthermoCohesin-hADH3] | C1328/8 |
| Cancer | Ecoli-pET28[6xHis-CthermoCohesin- | C1327/9 |
| | hHomosapienssyndecanbindingprotein] | |
| Cancer | Ecoli-pET28[6xHis-CthermoCohesin-hLOC552889] | C1326/10 |
| Cancer | Ecoli-pET28[Cohesin-hCyclinB1-v1-6xHis] | C708/11 |
| Cancer | Ecoli-pET28[Cohesin-hCyclinB1-v4-6xHis] | C736/12 |
| Cancer | Ecoli-pET28[Cohesin-hCyclinD1-6xHis] | C515/13 |
| Cancer | Ecoli-pET28[Cohesin-hgp100-PeptideA-6xHis] | C180/14 |
| Cancer | Ecoli-pET28[Cohesin-hMART-1-PeptideB-6xHis] | C181/15 |
| Cancer | Mam-cetHS-puro[SLAML-6xHis-Cohesin-hNY-ESO-1- | C1179/16 |
| | 6xHis] | |
| Cancer | Mam-cetHS-puro[SLAML-6xHis-Cohesin-hPSA] | C1163/17 |
| Cancer | Mam-cetHS-puro[SLAML-Cohesin-hSurvivin-6xHis] | C875/18 |
| Cancer | Mam-pCDM8[SLAML-Cohesin-hgp100] | C294/19 |
| Control | Ecoli-pET28[Cohesin-6xHis] | C21/20 |
| Control | Ecoli-pET28[Cohesin] | C189/21 |
| Hepatitis C | Ecoli-pET28[6xHis-CthermoCohesin-HCV-NS5B-Palm] | C1843/22 |
| Virus (HCV) | | |
| HCV | Ecoli-pET28[6xHis-CthermoCohesin-HCV-NS5B(1 + 2)] | C1641/23 |
| HCV | Ecoli-pET28[6xHis-CthermoCohesin-hHCVE1b] | C1920/24 |
| HCV | Ecoli-pET28[6xHis-CthermoCohesin-hHCVE2] | C1923/25 |
| HCV | Ecoli-pET28[6xHis-CthermoCohesin-ViralHCVprotease] | C1662/26 |
| HCV | Ecoli-pET28[CthermoCohesin-ViralHCVhelicase] | C1664/27 |
| HIV | Ecoli-pET28[6xHis-Cohesin-Pep-gag17] | C1079/28 |
| HIV | Ecoli-pET28[6xHis-Cohesin-Pep-gag253] | C1080/29 |
| HIV | Ecoli-pET28[6xHis-Cohesin-Pep-nef116] | C1078/30 |
| HIV | Ecoli-pET28[6xHis-Cohesin-Pep-nef66] | C1077/31 |
| HIV | Ecoli-pET28[6xHis-Cohesin-Pep-pol158] | C1081/32 |
| HIV | Ecoli-pET28[6xHis-Cohesin-Viralgag-p17-6xHis] | C1111/33 |
| HIV | Mam-cetHS-puro[SLAML-Cohesin-Viralgag-6xHis] | C492/34 |
| HIV | Mam-cetHS-puro[SLAML-Cohesin-ViralNef-6xHis] | C879/35 |
| Human | Ecoli-pET28[6xHis-Cohesin-HPV16E6] | C1435/36 |
| Papilloma Virus | | |
| (HPV) | | |
| HPV | Ecoli-pET28[6xHis-Cohesin-HPV16E7] | C1436/37 |
| HPV | Ecoli-pET28[6xHis-Cohesin-HPV16L2] | C1775/38 |
| HPV | Ecoli-pET28[6xHis-Cohesin-HPV18E7] | C1469/39 |
| HPV | Ecoli-pET28[6xHis-CthermoCohesin-HPV16E6-HPV16E7] | C1463/40 |
| HPV | Ecoli-pET28[6xHis-CthermoCohesin-HPV18E6-HPV18E7] | C1510/41 |
| HPV | Ecoli-pET28[6xHis-CthermoCohesin-HPV18E6] | C1496/42 |
| Influenza | Ecoli-pET28[6xHis-Cohesin-FluHA1-1c] | C2096/43 |
| Influenza | Ecoli-pET28[6xHis-Cohesin-FluHA3-1k] | C2039/44 |
| Influenza | Ecoli-pET28[6xHis-CthermoCohesin-FluNA-6xHis] | C1699/45 |
| Influenza | Ecoli-pET28[Cohesin-FluM1-6xHis] | C32/46 |
| Influenza | Ecoli-pET28[CthermoCohesin-FluHA1-1s-6xHis] | C1887/47 |
| Influenza | Ecoli-pET28[CthermoCohesin-FluNP-5-6xHis] | C943/48 |
| Influenza | Mam-cetHS-puro[6xHis-Cohesin-FluHA1-1u] | C1996/49 |
| Influenza | Mam-cetHS-puro[6xHis-Cohesin-FluHA3-1k] | C1998/50 |
| Influenza | Mam-cetHS-puro[6xHis-Cohesin-FluNP-1s] | C2043/51 |
| Influenza | Mam-cetHS-puro[Cohesin-Flex-v1-FluM2-1s-M2e-FluM2- | C2254/52 |
| | 1-M2e] | |
| Influenza | Mam-cetHS-puro[HA1-0-S-Cohesin-6xHis] | C1819/53 |
| Influenza | Mam-cetHS-puro[SLAML-6xHis-Cohesin-FluHAb-1] | C2297/54 |
| Influenza | Mam-cetHS-puro[SLAML-6xHis-Cohesin-FluM2-1s] | C1734/55 |
| Influenza | Mam-cetHS-puro[SLAML-6xHis-Cohesin-M2e] | C1977/56 |
| Influenza | Mam-cetHS-puro[SLAML-Cohesin-FluHA1-1-6xHis] | C489/57 |
| Influenza | Mam-cetHS-puro[SLAML-Cohesin-FluHA5-0-6xHis] | C543/58 |
| Influenza | Mam-cetHS-puro[SLAML-Cohesin-FluHA5-1-6xHis] | C490/59 |
| SIV | Mam-cetHS-puro[6xHis-Cohesin-ViralgagSIV-p17] | C1274/60 |
| SIV | Mam-cetHS-puro[6xHis-Cohesin-ViralgagSIV-p24] | C1273/61 |
| SIV | Mam-cetHS-puro[6xHis-Cohesin-ViralnefSIV] | C1275/62 |
| SIV | Mam-cetHS-puro[SLAML-6xHis-Cohesin-ViralenvSIV- | C1193/63 |
| | gp41-6xHis] | |
| Tuberculosis | Ecoli-pET28[6xHis-Cohesin-m.tbCFP10] | C1254/64 |
| Tuberculosis | Ecoli-pET28[6xHis-Cohesin-m.tbRv0125] | C1334/65 |
| Tuberculosis | Ecoli-pET28[6xHis-Cohesin-m.tbRv0570] | C1500/66 |
| Tuberculosis | Ecoli-pET28[6xHis-Cohesin-m.tbRv0577] | C1335/67 |
| Tuberculosis | Ecoli-pET28[6xHis-Cohesin-m.tbRv1626] | C1256/68 |
| Tuberculosis | Ecoli-pET28[6xHis-Cohesin-m.tbRv2875] | C1310/69 |
| Tuberculosis | Ecoli-pET28[6xHis-Cohesin-m.tbRv3044] | C1312/70 |
| Tuberculosis | Ecoli-pET28[6xHis-Cohesin-m.tbRv3478] | C1308/71 |
| Tuberculosis | Ecoli-pET28[6xHis-CthermoCohesin-m.tbAg85BDel41] | C2200/72 |
| Tuberculosis | Ecoli-pET28[6xHis-CthermoCohesin-m.tbMtb72f] | C2236/73 |
| Tuberculosis | Ecoli-pET28[6xHis-CthermoCohesin-m.tbRv0288] | C2202/74 |
| Tuberculosis | Ecoli-pET28[6xHis-CthermoCohesin-m.tbRv0475] | C2199/75 |
| Tuberculosis | Ecoli-pET28[6xHis-CthermoCohesin-m.tbRv1980] | C2197/76 |

Autoantigen: Ecoli-pET28[Cohesin-hGadB-6xHis] C914

(SEQ ID NO: 5)
MDPKGSLSWRILLFLSLAFELSYGLDDLDAVRIKVDTVNAKPGDTVR
IPVRFSGIPSKGIANCDFVYSYDPNVLEIIEIEPGDIIVDPNPDKSF
DTAVYPDRKIIVFLFAEDSGTGAYAITKDGVFATIVAKVKEGAPNGL
SVIKFVEVGGFANNDLVEQKTQFFDGGVNVGDTTEPATPTTPVTTPT
TTDDLDALEASQIGNMISIWVSHSIHTGNQHQSEPISNTNFLTEKAV
ASVKLAGNSSLCPINGWAVYSKDNSIRIGSKGDVFVIREPFISCSHL
ECRTFFLTQGALLNDKHSNGTVKDRSPHRTLMSCPVGEAPSPYNSRF
ESVAWSASACHDGTSWLTIGISGPDNGAVAVLKYNGIITDTIKSWRN
NILRTQESECACVNGSCFTVMTDGPSNGQASHKIFKMEKGKVVKSVE
LDAPNYHYEECSCYPNAGEITCVCRDNWHGSNRPWVSFNQNLEYQIG
YICSGVFGDNPRPNDGTGSCGPVSSNGAYGVKGFSFKYGNGVWIGRT
KSTNSRSGFEMIWDPNGWTETDSSFSVKQDIVAITDWSGYSGSFVQH
PELTGLDCIRPCFWVELIRGRPKESTIWTSGSSISFCGVNSDTVGWS
WPDGAELPFTIDKHHHHHH

Bacterial:Ecoli-pET28[6xHis-Cohesin-Flgn-1-Flgn-2] C1036

(SEQ ID NO: 6)
MGSSHHHHHHSSGLVPRGSHMASIERLSSGLRINSAKDDAAGQAIAN
RFTANIKGLTQASRNANDGISIAQTTEGALNEINNNLQRVRELAVQS
ANSTNSQSDLDSIQAEITQRLNEIDRVSGQTQFNGVKVLAQDNTLTI
QVGANDGETIDIDLKQINSQTLGLDSLNVQASQPELAEAAAKTTENP
LQKIDAALAQVDALRSDLGAVQNRFNSAITNLGNTVNNLSEARSRIE
DSDYATEVSNMSRAQILQAS

Bacterial toxin: Ecoli-pET28[Cohesin-P.aeruginosaPE38] C340

(SEQ ID NO: 7)
MDLDAVRIKVDTVNAKPGDTVNIPVRFSGIPSKGIANCDFVYSYDPNV
LEIIEIKPGELIVDPNPTKSFDTAVYPDRKMIVFLFAEDSGTGAYAIT
KDGVFATIVAKVKEGAPNGLSVIKFVEVGGFANNDLVEQKTQFFDGGV
NVGDTTEPATPTTPVTTPTTTDDLDAASEGGSLAALTAHQACHLPLET
FTRHRQPRGWEQLEQCGYPVQRLVALYLAARLSWNQVDQVIRNALASP
GSGGDLGEAIREQPEQARLALTLAAAESERFVRQGTGNDEAGAANGPA
DSGDALLERNYPTGAEFLGDGGDVSFSTRGTQNWTVERLLQAHRQLEE
RGYVFVGYHGTFLEAAQSIVFGGVRARSQDLDAIWRGFYIAGDPALAY
GYAQDQEPDARGRIRNGALLRVYVPRSSLPGFYRTSLTLAAPEAAGEV
ERLIGHPLPLRLDAITGPEEEGGRLETILGWPLAERTVVIPSAIPTDP
RNVGGDLDPSSIPDKEQAISALPDYASQPGKPPREDLK

Cancer Ecoli-pET28[6xHis-CthermoCohesin-hADH3] C1328

(SEQ ID NO: 8)
MGSSHHHHHHSSGLVPRGSHMASMDLDAVRIKVDTVNAKPGDTVNIP
VRFSGIPSKGIANCDFVYSYDPNVLEIIEIKPGELIVDPNPTKSFDT
AVYPDRKMIVFLFAEDSGTGAYAITKDGVFATIVAKVKEGAPNGLSV
IKFVEVGGFANNDLVEQKTQFFDGGVNVGDTTEPATPTTPVTTPTTT
DDLDAASELEVRRVRQAFLSGRSRPLRFRLQQLEALRRMVQEREKDI
LTAIAADLCKSEFNVYSQEVITVLGEIDFMLENLPEWVTAKPVKKNV
LTMLDEAYIQPQPLGVVLIIGAWNYPFVLTIQPLIGAIAAGNAVIIK
PSELSENTAKILAKLLPQYLDQDLYIVINGGVEETTELLKQRFDHIF
YTGNTAVGKIVMEAAAKHLTPVTLELGGKSPCYIDKDCDLDIVCRRI
TWGKYMNCGQTCIAPDYILCEASLQNQIVWKIKETVKEFYGENIKES
PDYERIINLRHFKRILSLLEGQKIAFGGETDEATRYIAPTVLTDVDP
KTKVMQEEIFGPILPIVPVKNVDEAINFINEREKPLALYVFSHNHKL
IKRMIDETSSGGVTGNDVIMHFTLNSFPFGGVGSSGMGAYHGKHSFD
TFSHQRPCLLKSLKREGANKLRYPPNSQSKVDWGKFFLLKRFNKEK

Cancer: Ecoli-pET28[6xHis-CthermoCohesin-hHomosapienssyndecanbindingprotein]

C1327

(SEQ ID NO: 9)
MGSSHHHHHHSSGLVPRGSHMASMDLDAVRIKVDTVNAKPGDTVNIP
VRFSGIPSKGIANCDFVYSYDPNVLEIIEIKPGELIVDPNPTKSFDT
AVYPDRKMIVFLFAEDSGTGAYAITKDGVFATIVAKVKEGAPNGLSV
IKFVEVGGFANNDLVEQKTQFFDGGVNVGDTTEPATPTTPVTTPTTT
DDLDAASSLYPSLEDLKVDKVIQAQTAFSANPANPAILSEASAPIPH
DGNLYPRLYPELSQYMGLSLNEEEIRANVAVVSGAPLQGQLVARPSS
INYMVAPVTGNDVGIRRAEIKQGIREVILCKDQDGKIGLRLKSIDNG
IFVQLVQANSPASLVGLRFGDQVLQINGENCAGWSSDKAHKVLKQAF
GEKITMTIRDRPFERTITMHKDSTGHVGFIFKNGKITSIVKDSSAAR
NGLLTEHNICEINGQNVIGLKDSQIADILSTSGTVVTITIMPAFIFE
HIIKRMAPSIMKSLMDHTIPEV

Cancer: Ecoli-pET28[6xHis-CthermoCohesin-hL00552889] C1326

(SEQ ID NO: 10)
MGSSHHHHHHSSGLVPRGSHMASMDLDAVRIKVDTVNAKPGDTVNIP
VRFSGIPSKGIANCDFVYSYDPNVLEIIEIKPGELIVDPNPTKSFDT
AVYPDRKMIVFLFAEDSGTGAYAITKDGVFATIVAKVKEGAPNGLSV
IKFVEVGGFANNDLVEQKTQFFDGGVNVGDTTEPATPTTPVTTPTTT
DDLDAASEEISLANLDTNKLEAIAQEIYVDLIEDSCLGFCFEVHRAV
KCGYFYLEFAETGSVKDFGIQPVEDKGACRLPLCSLPGEPGNGPDQQ
LQRSPPEFQ

Cancer: Ecoli-pET28[Cohesin-hCyclinBl-vl-6xHis] C708

(SEQ ID NO: 11)
MDLDAVRIKVDTVNAKPGDTVNIPVRFSGIPSKGIANCDFVYSYDPN
VLEIIEIKPGELIVDPNPTKSFDTAVYPDRKMIVFLFAEDSGTGAYA
ITKDGVFATIVAKVKEGAPNGLSVIKFVEVGGFANNDLVEQKTQFFD
GGVNVGDTTEPATPTTPVTTPTTTDDLDAASLEPEPEPEPEPVKEEK
LSPEPILVDTASPSPMETSGCAPAEEDLCQAFSDVILAVNDVDAEDG
ADPNLCSEYVKDIYAYLRQLEEEQAVRPKYLLGREVTGNMRAILIDW
LVQVQMKFRLLQETMYMTVSIIDRFMQNNCVPKKMLQLVGVTAMFIA
SKYEEMYPPEIGDFAFVTDNTYTKHQIRQMEMKILRALNFGLGRPLP
LHFLRRASKIGEVDVEQHTLAKYLMELTMLDYDMVHFPPSQIAAGAF
CLALKILDNGEWTPTLQHYLSYTEESLLPVMQHLAKNVVMVNQGLTK
HMTVKNKYATSKHAKISTLPQLNSALVQDLAKAVAKVHHHHHH

Cancer: Ecoli-pET28[Cohesin-hCyclinBl-v4-6xHis] C736

(SEQ ID NO: 12)
MDLDAVRIKVDTVNAKPGDTVNIPVRFSGIPSKGIANCDFVYSYDPNV
LEIIEIKPGELIVDPNPTKSFDTAVYPDRKMIVFLFAEDSGTGAYAIT
KDGVFATIVAKVKEGAPNGLSVIKFVEVGGFANNDLVEQKTQFFDGGV
NVGDTTEPATPTTPVTTPTTTDDLDAASLEMALRVTRNSKINAENKAK
INMAGAKRVPTAPAATSKPGLRPRTALGDIGNKVSEQLQAKMPMKKEA
KPSATGKVIDKKLPKPLEKVPMLVPVPVSEPVPEPEPEPEPEPVKEEK
LSPEPILVDTASPSPMETSGCAPAEEDLCQAFSDVILAVNDVDAEDGA
DPNLCSEYVKDIYAYLRQLEEEQAVRPKYLLGREHHHHHH

Cancer: Ecoli-pET28[Cohesin-hCyclinD1-6xHis] C515

(SEQ ID NO: 13)
MDLDAVRIKVDTVNAKPGDTVNIPVRFSGIPSKGIANCDFVYSYDPN
VLEIIEIKPGELIVDPNPTKSFDTAVYPDRKMIVFLFAEDSGTGAYA
ITKDGVFATIVAKVKEGAPNGLSVIKFVEVGGFANNDLVEQKTQFFD
GGVNVGDTTEPATPTTPVTTPTTTDDLDAASLEMEHQLLCCEVETIR
RAYPDANLLNDRVLRAMLKAEETCAPSVSYFKCVQKEVLPSMRKIVA
TWMLEVCEEQKCEEEVFPLAMNYLDRFLSLEPVKKSRLQLLGATCMF
VASKMKETIPLTAEKLCIYTDNSIRPEELLQMELLLVNKLKWNLAAM
TPHDFIEHFLSKMPEAEENKQIIRKHAQTFVALCATDVKFISNPPSM
VAAGSVVAAVQGLNLRSPNNFLSYYRLTRFLSRVIKCDPDCLRACQE
QIEALLESSLRQAQQNMDPKAAEEEEEEEEEVDLACTPTDVRDVDIH
HHHHH

Cancer: Ecoli-pET28[Cohesin-hgp100-PeptideA-6xHis] C180

(SEQ ID NO: 14)
MDLDAVRIKVDTVNAKPGDTVNIPVRFSGIPSKGIANCDFVYSYDPN
VLEIIEIKPGELIVDPNPTKSFDTAVYPDRKMIVFLFAEDSGTGAYA
ITKDGVFATIVAKVKEGAPNGLSVIKFVEVGGFANNDLVEQKTQFFD
GGVNVGDTTEPATPTTPVTTPTTTDDLDAARSAFTIMDQVPFSVSVS
ASRKGAAALEHHHHHH

Cancer: Ecoli-pET28[Cohesin-hMART-1-PeptideB-6xHis] C181

(SEQ ID NO: 15)
MDLDAVRIKVDTVNAKPGDTVNIPVRFSGIPSKGIANCDFVYSYDPN
VLEIIEIKPGELIVDPNPTKSFDTAVYPDRKMIVFLFAEDSGTGAYA
ITKDGVFATIVAKVKEGAPNGLSVIKFVEVGGFANNDLVEQKTQFFD
GGVNVGDTTEPATPTTPVTTPTTTDDLDAARTAEELAGIGILTVILG
ASRKGAAALEHHHHHH

Cancer: Mam-cetHS-puro[SLAML-6xHis-Cohesin-hNY-ESO-1-6xHis] C1179

(SEQ ID NO: 16)
LDITSHHHHHHDDLDAVRIKVDTVNAKPGDTVRIPVRFSGIPSKGIAN
CDFVYSYDPNVLEIIEIEPGDIIVDPNPDKSFDTAVYPDRKIIVFLFA
EDSGTGAYAITKDGVFATIVAKVKEGAPNGLSVIKFVEVGGFANNDLV
EQKTQFFDGGVNVGDTTEPATPTTPVTTPTTTDDLDAASQTPTNTISV
TPTNNSTPTNNSNPKPNPASMQAEGRGTGGSTGDADGPGGPGIPDGPG
GNAGGPGEAGATGGRGPRGAGAARASGPGGGAPRGPHGGAASGLNGCC
RCGARGPESRLLEFYLAMPFATPMEAELARRSLAQDAPPLPVPGVLLK
EFTVSGNILTIRLTAADHRQLQLSISSCLQQLSLLMWITQCFLPVFLA
QPPSGQRRHHHHHH

Cancer: Mam-cetHS-puro[SLAML-6xHis-Cohesin-hPSA] C1163

(SEQ ID NO: 17)
LDITSHHHHHHDDLDAVRIKVDTVNAKPGDTVRIPVRFSGIPSKGIA
NCDFVYSYDPNVLEHEIEPGDIIVDPNPDKSFDTAVYPDRKIIVFLF
AEDSGTGAYAITKDGVFATIVAKVKEGAPNGLSVIKFVEVGGFANND
LVEQKTQFFDGGVNVGDTTEPATPTTPVTTPTTTDDLDAASDTTEPA
TPTTPVTTPTTTLLAPLILSRIVGGWECEKHSQPWQVLVASRGRAVC
GGVLVHPQWVLTAAHCIRNKSVILLGRHSLFHPEDTGQVFQVSHSFP
HPLYDMSLLKNRFLRPGDDSSHDLMLLRLSEPAELTDAVKVMDLPTQ
EPALGTTCYASGWGSIEPEEFLTPKKLQCVDLHVISNDVCAQVHPQK
VTKFMLCAGRWTGGKSTCSGDSGGPLVCNGVLQGITSWGSEPCALPE
RPSLYTKVVHYRKWIKDTIVANP

Cancer: Mam-cetHS-puro[SLAML-Cohesin-hSurvivin-6xHis] C875

(SEQ ID NO: 18)
LDDLDAVRIKVDTVNAKPGDTVRIPVRFSGIPSKGIANCDFVYSYDP
NVLEIIEIEPGDIIVDPNPDKSFDTAVYPDRKIIVFLFAEDSGTGAY
AITKDGVFATIVAKVKEGAPNGLSVIKFVEVGGFANNDLVEQKTQFF
DGGVNVGDTTEPATPTTPVTTPTTTDDLDALEMGAPTLPPAWQPFLK
DHRISTFKNWPFLEGCACTPERMAEAGFIHCPTENEPDLAQCFFCFK
ELEGWEPDDDPIEEHKKHSSGCAFLSVKKQFEELTLGEFLKLDRERA
KNKIAKETNNKKKEFEETAKKVRRAIEQLAAMDHHHHHH

Cancer: Mam-pCDM8[SLAML-Cohesin-hgp100] C294

(SEQ ID NO: 19)
GLDDLDAVRIKVDTVNAKPGDTVRIPVRFSGIPSKGIANCDFVYSYDP
NVLEIIEIEPGDIIVDPNPDKSFDTAVYPDRKIIVFLFAEDSGTGAYA
ITKDGVFATIVAKVKEGAPNGLSVIKFVEVGGFANNDLVEQKTQFFDG
GVNVGDTTEPATPTTPVTTPTTTDDLDALEKVPRNQDWLGVSRQLRTA
WNRQLYPEWTEAQRLDCWRGGQVSLKVSNDGPTLIGANASFSIALNFP
GSQKVLPDGQVIWVNNTIINGSQVWGGQPVYPQETDDACIFPDGGPCP
SGSWSQKRSFVYVWKTWGQYWQVLGGPVSGLSIGTGRAMLGTHTMEVT
VYHRRGSRSYVPLAHSSSAFTITDQVPFSVSVSQLRALDGGNKHFLRN
QPLTFALQLHDPSGYLAEADLSYTWDFGDSSGTLISRAPVVTHTYLEP
GPVTAQVVLQAAIPLTSCGSSPVPGTTDGHRPTAEAPNTTAGQVPTTE
VVGTTPGQAPTAEPSGTTSVQVPTTEVISTAPVQMPTAESTGMTPEKV
PVSEVMGTTLAEMSTPEATGMTPAEVSIVVLSGTTAAQVTTTEWVETT
ARELPIPEPEGPDASSIMSTESITGSLGPLLDGTATLRLVKRQVPLDC
VLYRYGSFSVTLDIVQGIESAEILQAVPSGEGDAFELTVSCQGGLPKE
ACMEISSPGCQPPAQRLCQPVLPSPACQLVLHQILKGGSGTYCLNVSL
ADTNSLAVVSTQLIVPGILLTGQEAGLGQ

Control: Ecoli-pET28[Cohesin-6xHis] C21

(SEQ ID NO: 20)
MDLDAVRIKVDTVNAKPGDTVNIPVRFSGIPSKGIANCDFVYSYDPN
VLEIIEIKPGELIVDPNPTKSFDTAVYPDRKMIVFLFAEDSGTGAYA
ITKDGVFATIVAKVKEGAPNGLSVIKFVEVGGFANNDLVEQKTQFFD
GGVNVGDTTEPATPTTPVTTPTTTDDLDAASMTGGQQMGRDPNSSSV
DKLAAALEHHHHHH

Control: Ecoli-pET28[Cohesin] C189

(SEQ ID NO: 21)
MDLDAVRIKVDTVNAKPGDTVNIPVRFSGIPSKGIANCDFVYSYDPN
VLEIIEIKPGELIVDPNPTKSFDTAVYPDRKMIVFLFAEDSGTGAYA
ITKDGVFATIVAKVKEGAPNGLSVIKFVEVGGFANNDLVEQKTQFFD
GGVNVGDTTEPATPTTPVTTPTTTDDLDAASMTGGQQMGRDPNSSLR
PHSSTTTTTTEIRLLTKPERKLSWLLPPLSNN

Hepatitis C Virus: Ecoli-pET28[6xHis-CthermoCohesin-HCV-NS5B-Palm] C1843

(SEQ ID NO: 22)
MGSSHHHHHHSSGLVPRGSHMASMDLDAVRIKVDTVNAKPGDTVNIPV
RFSGIPSKGIANCDFVYSYDPNVLEIIEIKPGELIVDPNPTKSFDTAV
YPDRKMIVFLFAEDSGTGAYAITKDGVFATIVAKVKEGAPNGLSVIKF
VEVGGFANNDLVEQKTQFFDGGVNVGDTTEPATPTTPVTTPTTTDDLD
AASVLDSHYQDVLKEVKAAASKVKANALYDVVSKLPLAVMGSSYGFQY
SPGQRVEFLVQAWKSKKTPMGFSYDTRCFDSTVTESDIRTEEAIYQCC
DLDPQARVAIKSLTERLYVGRCRASGVLTTSCGNTLTCYIKARAACRA
AGLQDCTMLVCGDDLVVICESAGVQEDAASLRAFTEAMTRYSAPPGDP
PQPEYDLELITAS

Hepatitis C Virus: Ecoli-pET28[6xHis-CthermoCohesin-HCV-NS5B(1+2)] C1641

(SEQ ID NO: 23)
MGSSHHHHHHSSGLVPRGSHMASMDLDAVRIKVDTVNAKPGDTVNIP
VRFSGIPSKGIANCDFVYSYDPNVLEIIEIKPGELIVDPNPTKSFDT
AVYPDRKMIVFLFAEDSGTGAYAITKDGVFATIVAKVKEGAPNGLSV
IKFVEVGGFANNDLVEQKTQFFDGGVNVGDTTEPATPTTPVTTPTTT
DDLDAASSMSYSWTGALVTPCAAEAALVYSTTSRSACQRQKKVTFDR
LQVLDSHYQDVLKEVKAAASKVKANLLSVEEACSLTPPHSAKSKFGY
GAKDVRCHARKAVNHINSVWKDLLEDSVTPIDTTIMAKNEVFCVQPE
KGGRKPARLIVFPDLGVRVCEKMALYDVVSKLPLAVMGSSYGFQYSP
GQRVEFLVQAWKSKKTPMGFSYDTRCFDSTVTESDIRTEEAIYQCCD
LDPQARVAIKSLTERLYVGGPLTNSRGENCGYRRCRASGVLTTSCGN
TLTCYIKARAACRAAGLQDCTMLVCGDDLVVICESAGVQEDAASLRA
FTEAMTRYSAPPGDPPQPEYDLELITSCSAS

Hepatitis C Virus: Ecoli-pET28[6xHis-CthermoCohesin-hHCVE1b] C1920

(SEQ ID NO: 24)
MGSSHHHHHHSSGLVPRGSHMASMDLDAVRIKVDTVNAKPGDTVNIP
VRFSGIPSKGIANCDFVYSYDPNVLEIIEIKPGELIVDPNPTKSFDT
AVYPDRKMIVFLFAEDSGTGAYAITKDGVFATIVAKVKEGAPNGLSV
IKFVEVGGFANNDLVEQKTQFFDGGVNVGDTTEPATPTTPVTTPTTT
DDLDAASVGQLFTFSPRRHWTTQDCNCSIYPGHITGHRMAWDMMMNW
SPTTAVVAQLLRIPQAILDMIAGAS

Hepatitis C Virus: Ecoli-pET28[6xHis-CthermoCohesin-hHCVE2] C1923

(SEQ ID NO: 25)
MGSSHHHHHHSSGLVPRGSHMASMDLDAVRIKVDTVNAKPGDTVNIP
VRFSGIPSKGIANCDFVYSYDPNVLEIIEIKPGELIVDPNPTKSFDT
AVYPDRKMIVFLFAEDSGTGAYAITKDGVFATIVAKVKEGAPNGLSV
IKFVEVGGFANNDLVEQKTQFFDGGVNVGDTTEPATPTTPVTTPTTT
DDLDAASETHVTGGSAARTTAGLAGLFTPGAKQNIQLINTNGSWHIN
RTALNCNDSLNTGWVAGLFYYHKFNSSGCPERLASCRPLTDFDQGWG
PISYANGSGPDQRPYCWHYPPKPCGIVPAKSVCGPVYCFTPSPVVVG
TTDRSGAPTYNWGENDTDVFVLNNTRPPLGNWFGCTWMNSTGFTKVC
GAPPCVIGGVGNNTLHCPTDCFRKHPEATYSRCGSGPWITPRCLVDY
PYRLWHYPCTINYTIFKIRMYVGGVEHRLEAACNWTRGERCDLEDRD
RSELSPLLLSTTQWQVLPCSFTTLPALSTGLIHLHQNIVDVQYLYGV
GSSIASWAIKWEYVVLLFLLAS

Hepatitis C Virus: Ecoli-pET28[6xHis-CthermoCohesin-ViralHCVprotease] c1662

(SEQ ID NO: 26)
MGSSHHHHHHSSGLVPRGSHMASMDLDAVRIKVDTVNAKPGDTVNIP
VRFSGIPSKGIANCDFVYSYDPNVLEIIEIKPGELIVDPNPTKSFDT
AVYPDRKMIVFLFAEDSGTGAYAITKDGVFATIVAKVKEGAPNGLSV
IKFVEVGGFANNDLVEQKTQFFDGGVNVGDTTEPATPTTPVTTPTTT
DDLDAASAPITAYAQQTRGLLGCIITSLTGRDKNQVEGEVQIVSTAA
QTFLATCINGVCWTVYHGAGTRTIASPKGPVIQMYTNVDQDLVGWPA
PQGARSLTPCTCGSSDLYLVTRHADVIPVRRRGDSRGSLLSPRPISY
LKGSSGGPLLCPAGHAVGIFRAAVCTRGVAKAVDFIPVENLETTMRS
PVFTDNSSPPAVPQSASSTQPEFAA

Hepatitis C Virus: Ecoli-pET28[CthermoCohesin-ViralHCVhelicase] C1664

(SEQ ID NO: 27)
MGSSHHHHHHSSGLVPRGSHMASMDLDAVRIKVDTVNAKPGDTVNIP
VRFSGIPSKGIANCDFVYSYDPNVLEIIEIKPGELIVDPNPTKSFDT
AVYPDRKMIVFLFAEDSGTGAYAITKDGVFATIVAKVKEGAPNGLSV
IKFVEVGGFANNDLVEQKTQFFDGGVNVGDTTEPATPTTPVTTPTTT
DDLDAASFQVAHLHAPTGSGKSTKVPAAYAAQGYKVLVLNPSVAATL
GFGAYMSKAHGIDPNIRTGVRTITTGSPITYSTYGKFLADGGCSGGA
YDIIICDECHSTDATSILGIGTVLDQAETAGARLVVLATATPPGSVT
VPHPNIEEVALSTTGEIPFYGKAIPLEVIKGGRHLIFCHSKKKCDEL
AAKLVALGINAVAYYRGLDVSVIPTSGVVVVVATDALMTGFTGDFDS
VIDCNTCVTQTVDFSLDPTFTIETTTLPQDAVSRTQRRGRTGRGKPG
IYRFVAPGERPSGMFDSSVLCECYDAGCAWYELTPAETTVRLRAYMN
TPGLPVCQDHLEFWEGVFTGLTHIDAHFLSQTKQSGENLPYLVAYQA
TVCARAQAPPPSWDQMWKCLIRLKPTLHGPTPLLYRLGAVQNEVTLT
HPITKYIMTCMSADLEVVTAAALEHHHHHH

HIV: Ecoli-pET28[6xHis-Cohesin-Pep-gag17] C1079

(SEQ ID NO: 28)
MGSSHHHHHHSSGLVPRGSHMASMDLDAVRIKVDTVNAKPGDTVNIP
VRFSGIPSKGIANCDFVYSYDPNVLEIIEIKPGELIVDPNPTKSFDT
AVYPDRKMIVFLFAEDSGTGAYAITKDGVFATIVAKVKEGAPNGLSV
IKFVEVGGFANNDLVEQKTQFFDGGVNVGDTTEPATPTTPVTTPTTT
DDLDAASEKIRLRPGGKKKYKLKHIVAS

HIV: Ecoli-pET28[6xHis-Cohesin-Pep-gag253] C1080

(SEQ ID NO: 29)
MGSSHHHHHHSSGLVPRGSHMASMDLDAVRIKVDTVNAKPGDTVNIP
VRFSGIPSKGIANCDFVYSYDPNVLEIIEIKPGELIVDPNPTKSFDT
AVYPDRKMIVFLFAEDSGTGAYAITKDGVFATIVAKVKEGAPNGLSV
IKFVEVGGFANNDLVEQKTQFFDGGVNVGDTTEPATPTTPVTTPTTT
DDLDAASNPPIPVGEIYKRWIILGLNKIVRMYSPTSILDAS

HIV: Ecoli-pET28[6xHis-Cohesin-Pep-nef116] C1078

(SEQ ID NO: 30)
MGSSHHHHHHSSGLVPRGSHMASMDLDAVRIKVDTVNAKPGDTVNIP
VRFSGIPSKGIANCDFVYSYDPNVLEIIEIKPGELIVDPNPTKSFDT
AVYPDRKMIVFLFAEDSGTGAYAITKDGVFATIVAKVKEGAPNGLSV
IKFVEVGGFANNDLVEQKTQFFDGGVNVGDTTEPATPTTPVTTPTTT
DDLDAASHTQGYFPDWQNYTPGPGVRYPLTFGWLYKLAS

HIV: Ecoli-pET28[6xHis-Cohesin-Pep-nef66] C1077

(SEQ ID NO: 31)
MGSSHHHHHHSSGLVPRGSHMASMDLDAVRIKVDTVNAKPGDTVNIP
VRFSGIPSKGIANCDFVYSYDPNVLEIIEIKPGELIVDPNPTKSFDT
AVYPDRKMIVFLFAEDSGTGAYAITKDGVFATIVAKVKEGAPNGLSV
IKFVEVGGFANNDLVEQKTQFFDGGVNVGDTTEPATPTTPVTTPTTT
DDLDAASVGFPVTPQVPLRPMTYKAAVDLSHFLKEKGGLAS

HIV: Ecoli-pET28[6xHis-Cohesin-Pep-pol158] C1081

(SEQ ID NO: 32)
MGSSHHHHHHSSGLVPRGSHMASMDLDAVRIKVDTVNAKPGDTVNIP
VRFSGIPSKGIANCDFVYSYDPNVLEIIEIKPGELIVDPNPTKSFDT
AVYPDRKMIVFLFAEDSGTGAYAITKDGVFATIVAKVKEGAPNGLSV
IKFVEVGGFANNDLVEQKTQFFDGGVNVGDTTEPATPTTPVTTPTTT
DDLDAASAIFQSSMTKILEPFRKQNPDIVIYQYMDDLYAS

HIV: Ecoli-pET28[6xHis-Cohesin-Viralgag-p17-6xHis] C1111

(SEQ ID NO: 33)
MGSSHHHHHHSSGLVPRGSHMASMDLDAVRIKVDTVNAKPGDTVNI
PVRFSGIPSKGIANCDFVYSYDPNVLEIIEIKPGELIVDPNPTKSF
DTAVYPDRKMIVFLFAEDSGTGAYAITKDGVFATIVAKVKEGAPNG
LSVIKFVEVGGFANNDLVEQKTQFFDGGVNVGDTTEPATPTTPVTT
PTTTDDLDAASLEMGARASILSGGELDRWEKIRLRPGGKKKYKLKH
IVWASRELERFAVNPGLLETSEGCRQILGQLQPSLQTGSEELRSLY
NTVATLYCVHQRIEIKDTKEALDKIEEEQNKSVDHHHHHH

HIV: Mam-cetHS-puro[SLAML-Cohesin-Viralgag-6xHis] C492

(SEQ ID NO: 34)
LDDLDAVRIKVDTVNAKPGDTVRIPVRFSGIPSKGIANCDFVYSYDP
NVLEIIEIEPGDIIVDPNPDKSFDTAVYPDRKIIVFLFAEDSGTGAY
AITKDGVFATIVAKVKEGAPNGLSVIKFVEVGGFANNDLVEQKTQFF
DGGVNVGDTTEPATPTTPVTTPTTTDDLDALEVHQAISPRTLNAWVK
VVEEKAFSPEVIPMFSALSEGATPQDLNTMLNTVGGHQAAMQMLKET
INEEAAEWDRVHPVHAGPIAPGQMREPRGSDIAGTTSTLQEQIGWMT
NNPPIPVGEIYKRWIILGLNKIVRMYSPTSILDIRQGPKEPFRDYVD
RFYKTLRAEQASQEVKNWMTETLLVQNANPDCKTILKALGPAATLEE
MMTACQGVGHHHHHH

HIV: Mam-cetHS-puro[SLAML-Cohesin-ViralNef-6xHis] C879

(SEQ ID NO: 35)
LDDLDAVRIKVDTVNAKPGDTVRIPVRFSGIPSKGIANCDFVYSYDP
NVLEIIEIEPGDIIVDPNPDKSFDTAVYPDRKIIVFLFAEDSGTGAY
AITKDGVFATIVAKVKEGAPNGLSVIKFVEVGGFANNDLVEQKTQFF
DGGANVGDTTEPATPTTPVTTPTTTDDLDAASMGGKWSKRSVVGWPT
VRERMRRAEPAADGVGAVSRDLEKHGAITSSNTAANNADCAWLEAQE
EEEVGFPVRPQVPLRPMTYKGALDLSHFLKEKGGLEGLIYSQKRQDI
LDLWVYHTQGYFPDWQNYTPGPGIRYPLTFGWCFKLVPVEPEKVEEA
NEGENNSLLHPMSLHGMDDPEREVLVWKFDSRLAFHHMARELHPEYY
KDCHHHHHH

Human Papilloma Virus: Ecoli-pET28[6xHis-Cohesin-HPV16E6] C1435

(SEQ ID NO: 36)
MGSSHHHHHHSSGLVPRGSHMASMDLDAVRIKVDTVNAKPGDTVNIP
VRFSGIPSKGIANCDFVYSYDPNVLEIIEIKPGELIVDPNPTKSFDT
AVYPDRKMIVFLFAEDSGTGAYAITKDGVFATIVAKVKEGAPNGLSV
IKFVEVGGFANNDLVEQKTQFFDGGVNVGDTTEPATPTTPVTTPTTT
DDLDAASMHQKRTAMFQDPQERPRKLPQLCTELQTTIHDIILECVYC
KQQLLRREVGDFAFRDLCIVYRDGNPYAVCDKCLKFYSKISEYRHYC
YSVYGTTLEQQYNKPLCDLLIRCINCQKPLCPEAS

Human Papilloma Virus: Ecoli-pET28[6xHis-Cohesin-HPV16E7] C1436

(SEQ ID NO: 37)
MGSSHHHHHHSSGLVPRGSHMASMDLDAVRIKVDTVNAKPGDTVNIP
VRFSGIPSKGIANCDFVYSYDPNVLEIIEIKPGELIVDPNPTKSFDT
AVYPDRKMIVFLFAEDSGTGAYAITKDGVFATIVAKVKEGAPNGLSV
IKFVEVGGFANNDLVEQKTQFFDGGVNVGDTTEPATPTTPVTTPTTT
DDLDAASMHGDTPTLHEYMLDLOPETTDLYGYGOLNDSSEEEDEIDG
PAGQAEPDRAHYNIVTFCCKA

Human Papilloma Virus: Ecoli-pET28[6xHis-Cohesin-HPV16L2] C1775

(SEQ ID NO: 38)
MGSSHHHHHHSSGLVPRGSHMASMDLDAVRIKVDTVNAKPGDTVNI
PVRFSGIPSKGIANCDFVYSYDPNVLEIIEIKPGELIVDPNPTKSF
DTAVYPDRKMIVFLFAEDSGTGAYAITKDGVFATIVAKVKEGAPNG
LSVIKFVEVGGFANNDLVEQKTQFFDGGVNVGDTTEPATPTTPVTT
PTTTDDLDAASSGTGGRTGYIPLGTRPPTATDTLAPVRPPLTVDPV
GPSDPSIVSLVEETSFIDAGAPTSVPSIPPDVSGFSITTSTDTTPA
ILDINNTVTTVTTHNNPTFTDPSVLQPPTPAETGGHFTLSSSTIST
HNYEEIPMDTFIVSTNPNTVTSSTPIPGSRPVARLGLYSRTTQQVK
VVDPAFVTTPTKLITYDNPAYEGIDVDNTLYFSSNDNSINIAPDPD
FLDIVALHRPALTSRRTGIRYSRIGNKQTLRTRSGKSIGAKVHYYY
DLSTIDPAEEIELQTITPSTYTTTSHAASPTSINNGLYDIYAAS

Human Papilloma Virus: Ecoli-pET28[6xHis-Cohesin-HPV18E7] C1469

(SEQ ID NO: 39)
MGSSHHHHHHSSGLVPRGSHMASMDLDAVRIKVDTVNAKPGDTVNIP
VRFSGIPSKGIANCDFVYSYDPNVLEIIEIKPGELIVDPNPTKSFDT
AVYPDRKMIVFLFAEDSGTGAYAITKDGVFATIVAKVKEGAPNGLSV
IKFVEVGGFANNDLVEQKTQFFDGGVNVGDTTEPATPTTPVTTPTTT
DDLDAASMHGPKATLQDIVLHLEPQNEIPVDLLGHGQLSDSEEENDE
IDGVNHQHLPARRAEPQRHTMLCMCCKAS

Human Papilloma Virus: Ecoli-pET28[6xHis-CthermoCohesin-HPV16E6-HPV16E7] C 1463

(SEQ ID NO: 40)
GSSHHHHHHSSGLVPRGSHMASMDLDAVRIKVDTVNAKPGDTVNIPVR
FSGIPSKGIANCDFVYSYDPNVLEIIEIKPGELIVDPNPTKSFDTAVY
PDRKMIVFLFAEDSGTGAYAITKDGVFATIVAKVKEGAPNGLSVIKFV
EVGGFANNDLVEQKTQFFDGGVNVGDTTEPATPTTPVTTPTTTDDLDA
ASMHQKRTAMFQDPQERPRKLPQLCTELQTTIHDIILECVYCKQQLLR
REVGDFAFRDLCIVYRDGNPYAVCDKCLKFYSKISEYRHYCYSVYGTT
LEQQYNKPLCDLLIRCINCQKPLCPEASMHGDTPTLHEYMLDLQPETT
DLYGYGQLNDSSEEEDEIDGPAGQAEPDRAHYNIVTFCCKAS

Human Papilloma Virus: Ecoli-pET28[6xHis-CthermoCohesin-HPV18E6-HPV18E7] C1510

(SEQ ID NO: 41)
GSSHHHHHHSSGLVPRGSHMASMDLDAVRIKVDTVNAKPGDTVNIP
VRFSGIPSKGIANCDFVYSYDPNVLEIIEIKPGELIVDPNPTKSFD
TAVYPDRKMIVFLFAEDSGTGAYAITKDGVFATIVAKVKEGAPNGL
SVIKFVEVGGFANNDLVEQKTQFFDGGVNVGDTTEPATPTTPVTTP
TTTDDLDAASMARFEDPTRRPYKLPDLCTELNTSLQDIEITCVYCK
TVLELTEVGEFAFKDLFVVYRDSIPHAACHKCIDFYSRIRELRHYS
DSVYGDTLEKLTNTGLYNLLIRCLRCQKPLNPASMHGPKATLQDIV
LHLEPQNEIPVDLLGHGQLSDSEEENDEIDGVNHQHLPARRAEPQR
HTMLCMCCKAS

Human Papilloma Virus: Ecoli-pET28[6xHis-CthermoCohesin-HPV18E6] C1496

(SEQ ID NO: 42)
GSSHHHHHHSSGLVPRGSHMASMDLDAVRIKVDTVNAKPGDTVNIP
VRFSGIPSKGIANCDFVYSYDPNVLEIIEIKPGELIVDPNPTKSFD
TAVYPDRKMIVFLFAEDSGTGAYAITKDGVFATIVAKVKEGAPNGL
SVIKFVEVGGFANNDLVEQKTQFFDGGVNVGDTTEPATPTTPVTTP
TTTDDLDAASMARFEDPTRRPYKLPDLCTELNTSLQDIEITCVYCK
TVLELTEVGEFAFKDLFVVYRDSIPHAACHKCIDFYSRIRELRHYS
DSVYGDTLEKLTNTGLYNLLIRCLRCQKPLNPAS

Influenza: Ecoli-pET28[6xHis-Cohesin-F1uHA1-1c] C2096

(SEQ ID NO: 43)
MGSSHHHHHHSSGLVPRGSHMASMDLDAVRIKVDTVNAKPGDTVNIP
VRFSGIPSKGIANCDFVYSYDPNVLEIIEIKPGELIVDPNPTKSFDT
AVYPDRKMIVFLFAEDSGTGAYAITKDGVFATIVAKVKEGAPNGLSV
IKFVEVGGFANNDLVEQKTQFFDGGVNVGDTTEPATPTTPVTTPTTT
DDLDAASDTTEPATPTTPVTTDTICIGYHANNSTDTVDTVLEKNVTV
THSVNLLEDKHNGKLCKLRGVAPLHLGKCNIAGWILGNPECESLSTA
SSWSYIVETSSSDNGTCYPGDFIDYEELREQLSSVSSFERFEIFPKT
SSWPNHDSNKGVTAACPHAGAKSFYKNLIWLVKKGNSYPKLSKSYIN
DKGKEVLVLWGIHHPSTSDDQQSLYQNADAYVFVGSSRYSKKFKPEI
AIRPKVRDQEGRMNYYWTLVEPGDKITFEATGNLVVPRYAFAMERNA
GSGIIISDTPVHDCNTTCQTPKGAINTSLPFQNIQPITIGKCPKYVK
STKLRL

Influenza: Ecoli-pET28[6xHis-Cohesin-F1uHA3-1k] C2039

(SEQ ID NO: 44)
MGSSHHHHHHSSGLVPRGSHMASMDLDAVRIKVDTVNAKPGDTVNIP
VRFSGIPSKGIANCDFVYSYDPNVLEIIEIKPGELIVDPNPTKSFDT
AVYPDRKMIVFLFAEDSGTGAYAITKDGVFATIVAKVKEGAPNGLSV
IKFVEVGGFANNDLVEQKTQFFDGGVNVGDTTEPATPTTPVTTPTTT
DDLDAASDTTEPATPTTPVTTATLCLGHHAVPNGTLVKTITDDQIEV
TNATELVQSSSTGKICNNPHRILDGIDCTLIDALLGDPHCDVFQDET
WDLFVERSKAFSNCYPYDVPDYASLRSLVALSGTLEFITEGFTWTGV
TQNGGSNACKRGPGSGFFSRLNWLTKSGSTYPVLNVTMPNNDNFDKL
YIWGVHHPSTNQEQTSLYVQASGRVTVSTRRSQQTIIPNIGSRPWVR
GLSSRISIYWTIVKPGDVLVINSNGNLIAPRGYFKMRTGKSSIMRSD
APTDTCISECITPNGSIPNDKPFQNVNKITYGACPKYVKQNTLKLA

Influenza: Ecoli-pET28[6xHis-CthermoCohesin-FluNA-6xHis] C1699

(SEQ ID NO: 45)
MGSSHHHHHHSSGLVPRGSHMASMDLDAVRIKVDTVNAKPGDTVNI
PVRFSGIPSKGIANCDFVYSYDPNVLEIIEIKPGELIVDPNPTKSF
DTAVYPDRKMIVFLFAEDSGTGAYAITKDGVFATIVAKVKEGAPNG
LSVIKFVEVGGFANNDLVEQKTQFFDGGVNVGDTTEPATPTTPVTT
PTTTDDLDAASQIGNMISIWVSHSIHTGNQHQSEPISNTNFLTEKA
VASVKLAGNSSLCPINGWAVYSKDNSIRIGSKGDVFVIREPFISCS
HLECRTFFLTQGALLNDKHSNGTVKDRSPHRTLMSCPVGEAPSPYN
SRFESVAWSASACHDGTSWLTIGISGPDNGAVAVLKYNGIITDTIK
SWRNNILRTQESECACVNGSCFTVMTDGPSNGQASHKIFKMEKGKV
VKSVELDAPNYHYEECSCYPNAGEITCVCRDNWHGSNRPWVSFNQN
LEYQIGYICSGVFGDNPRPNDGTGSCGPVSSNGAYGVKGFSFKYGN
GVWIGRTKSTNSRSGFEMIWDPNGWTETDSSFSVKQDIVAITDWSG
YSGSFVQHPELTGLDCIRPCFWVELIRGRPKESTIWTSGSSISFCG
VNSDTVGWSWPDGAELPFTIDKHHHHHH

Influenza: Ecoli-pET28[Cohesin-FluM1-6xHis] C32

(SEQ ID NO: 46)
MDLDAVRIKVDTVNAKPGDTVNIPVRFSGIPSKGIANCDFVYSYDP
NVLEIIEIKPGELIVDPNPTKSFDTAVYPDRKMIVFLFAEDSGTGA
YAITKDGVFATIVAKVKEGAPNGLSVIKFVEVGGFANNDLVEQKTQ
FFDGGVNVGDTTEPATPTTPVTTPTTTDDLDAASLLTEVETYVLSI
IPSGPLKAEIAQRLEDVFAGKNTDLEVLMEWLKTRPILSPLTKGIL
GFVFTLTVPSERGLQRRRFVQNALNGNGDPNNMDKAVKLYRKLKRE
ITFHGAKEIALSYSAGALASCMGLIYNRMGAVTTEVAFGLVCATCE
QIADSQHRSHRQMVTTTNPLIRHENRMVLASTTAKAMEQMAGSSEQ
AAEAMDIASQARQMVQAMRTIGTHPSSSAGLKDDLLENLQAYQKRM
GVQMQRFKLEHHHHHH

Influenza: Ecoli-pET28[CthermoCohesin-F1uHA1-1s-6xHis] C1887

(SEQ ID NO: 47)
HHHHHHSSGLVPRGSHMASMDLDAVRIKVDTVNAKPGDTVNIPVRFS
GIPSKGIANCDFVYSYDPNVLEIIEIKPGELIVDPNPTKSFDTAVYP
DRKMIVFLFAEDSGTGAYAITKDGVFATIVAKVKEGAPNGLSVIKFV
EVGGFANNDLVEQKTQFFDGCGVNVGDTTEPATPTTPVTTPTTTDDL
DAASDTLCIGYHANNSTDTVDTVLEKNVTVTHSVNLLEDKHNGKLCK
LRGVAPLHLGKCNIAGWILGNPECESLSTASSWSYIVETPSSDNGTC
YPGDFIDYEELREQLSSVSSFERFEIFPKTSSWPNHDSNKGVTAACP
HAGAKSFYKNLIWLVKKGNSYPKLSKSYINDKGKEVLVLWGIHHPST
SADQQSLYQNADTYVFVGSSRYSKKFKPEIAIRPKVRDQEGRMNYYW
TLVEPGDKITFEATGNLVVPRYAFAMERNAGSGUISDTPVHDCNTTC
QTPKGAINTSLPFQNIHPITIGKCPKYVKSTKLRLAHHHHHH

Influenza: Ecoli-pET28[CthermoCohesin-FluNP-5-6xHis] C943

(SEQ ID NO: 48)
MDLDAVRIKVDTVNAKPGDTVNIPVRFSGIPSKGIANCDFVYSYDPN
VLEIIEIKPGELIVDPNPTKSFDTAVYPDRKMIVFLFAEDSGTGAYA
ITKDGVFATIVAKVKEGAPNGLSVIKFVEVGGFANNDLVEQKTQFFD
GGVNVGDTTEPATPTTPVTTPTTTDDLDAASMASQGTKRSYEQMETG
GERQNATEIRASVGRMVSGIGRFYIQMCTELKLSDYEGRLIQNSITI
ERMVLSAFDERRNRYLEEHPSAGKDPKKTGGPIYRRRDGKWVRELIL
YDKEEIRRIWRQANNGEDATAGLTHLMIWHSNLNDATYQRTRALVRT
GMDPRMCSLMQGSTLPRRSGAAGAAVKGVGTMVMELIRMIKRGINDR
NFWRGENGRRTRIAYERMCNILKGKFQTAAQRAMMDQVRESRNPGNA
EIEDLIFLARSALILRGSVAHKSCLPACVYGLAVASGYDFEREGYSL
VGIDPFRLLQNSQVFSLIRPNENPAHKSQLVWMACHSAAFEDLRVSS
FIRGTRVVPRGQLSTRGVQIASNENMEAMDSNTLELRSRYWAIRTRS
GGNTNQQRASAGQISVQPTFSVQRNLPFERATIMAAFTGNTEGRTSD
MRTEIIRMMESARPEDVSFQGRGVFELSDEKATNPIVPSFDMNNEGS
YFFGDNAEEYDNHHHHHH

Influenza: Mam-cetHS-puro[6xHis-Cohesin-F1uHA1-1u] C1996

(SEQ ID NO: 49)
LDITSHHHHHHDDLDAVRIKVDTVNAKPGDTVRIPVRFSGIPSKGIA
NCDFVYSYDPNVLEHEIEPGDIIVDPNPDKSFDTAVYPDRKIIVFLF
AEDSGTGAYAITKDGVFATIVAKVKEGAPNGLSVIKFVEVGGFANND
LVEQKTQFFDGGVNVGDTTEPATPTTPVTTPTTTDDLDAASDTTEPA
TPTTPVTTDTICIGYHANNSTDTVDTVLEKNVTVTHSVNLLEDSHNG
KLCRLKGIAPLQLGKCSIAGWILGNPECESLVSKKSWSYIAETPNSE
NGTCYPGYFADYEELREQLSSVSSFERFEIFPKERSWPKHNVTRGVT
ASCSHKGKSSFYRNLLWLTEKNGSYPNLSKSYVNNKEKEVLVLWGVH
HPSNIEDQKTIYRKENAYVSVVSSNYNRRFAPEIAERPKVRGQAGRI
NYYWTLLEPGDTIIFEANGNLIAPWHAFALNRGFGSGIITSNASMDE
CDTKCQTPQGAINSSLPFQNIHPVTIGECPKYVRSTKLRMV

Influenza: Mam-cetHS-puro[6xHis-Cohesin-F1uHA3-1k] C1998

(SEQ ID NO: 50)
LDITSHHHHHHDDLDAVRIKVDTVNAKPGDTVRIPVRFSGIPSKGIA
NCDFVYSYDPNVLEIIEIEPGDIIVDPNPDKSFDTAVYPDRKIIVFL
FAEDSGTGAYAITKDGVFATIVAKVKEGAPNGLSVIKFVEVGGFANN
DLVEQKTQFFDGGVNVGDTTEPATPTTPVTTPTTTDDLDAASDTTEP
ATPTTPVTTATLCLGHHAVPNGTLVKTITDDQIEVTNATELVQSSST
GKICNNPHRILDGIDCTLIDALLGDPHCDVFQNETWDLFVERSKAFS
NCYPYDVPDYASLRSLVASSGTLEFITEGFTWTGVTQNGGSNACKRG
PGSGFFSRLNWLTKSGSTYPVLNVTMPNNDNFDKLYIWGVHHPSTNQ
EQTSLYVQASGRVTVSTRRSQQTIIPNIGSRPWVRGLSSRISIYWTI
VKPGDVLVINSNGNLIAPRGYFKMRTGKSSIMRSDAPIDTCISECIT
PNGSIPNDKPFQNVNKITYGACPKYVKQNTLKLA

Influenza: Mam-cetHS-puro[6xHis-Cohesin-FluNP-1s] C2043

(SEQ ID NO: 51)
LDITSHHHHHHDDLDAVRIKVDTVNAKPGDTVRIPVRFSGIPSKGIA
NCDFVYSYDPNVLEIIEIEPGDIIVDPNPDKSFDTAVYPDRKIIVFL
FAEDSGTGAYAITKDGVFATIVAKVKEGAPNGLSVIKFVEVGGFANN
DLVEQKTQFFDGGVNVGDTTEPATPTTPVTTPTTTDDLDAASMASQG
TKRSYEQMETGGERQDATEIRASVGRMIGGIGRFYIQMCTELKLSDY
DGRLIQNSITIERMVLSAFDERRNKYLEEHPSAGKDPKKTGGPIYRR
VDGKWMRELILYDKEEIRRVWRQANNGEDATAGLTHIMIWHSNLNDA
TYQRTRALVRTGMDPRMCSLMQGSTLPRRSGAAGAAVKGVGTIAMEL
IRMIKRGINDRNFWRGENGRRTRVAYERMCNILKGKFQTAAQRAMMD
QVRESRNPGNAEIEDLIFLARSALILRGSVAHKSCLPACVYGLAVAS
GHDFEREGYSLVGIDPFKLLQNSQVVSLMRPNENPAHKSQLVWMACH
SAAFEDLRVSSFIRGKKVIPRGKLSTRGVQIASNENVETMDSNTLEL
RSRYWAIRTRSGGNTNQQKASAGQISVQPTFSVQRNLPFERATVMAA
FSGNNEGRTSDMRTEVIRMMESAKPEDLSFQGRGVFELSDEKATNPI
VPSFDMSNEGSYFFGDNAEEYDSASHHHHHH

Influenza: Mam-cetHS-puro[Cohesin-Flex-v1-F1uM2-1s-M2e-F1uM2-1-M2e C2254

(SEQ ID NO: 52)
LDITSHHHHHHDDLDAVRIKVDTVNAKPGDTVRIPVRFSGIPSKGIA
NCDFVYSYDPNVLEIIEIEPGDIIVDPNPDKSFDTAVYPDRKIIVFL
FAEDSGTGAYAITKDGVFATIVAKVKEGAPNGLSVIKEVEVGGFANN
DLVEQKTQFFDGGVNVGDTTEPATPTTPVTTPTTTDDLDAASQTPTN
TISVTPTNNSTPTNNSNPKPNPASLLTEVETPTRSEWECRCSDSSDP
ASLLTEVETPIRNEWGCRCNGSSDPAS

Influenza: Mam-cetHS-puro[HA1-0-S-Cohesin-6xHis] C1819

(SEQ ID NO: 53)
DTLCIGYHANNSTDTVDTVLEKNVTVTHSVNLLEDKHNGKLCKLRGVA
PLHLGKCNIAGWILGNPECESLSTASSWSYIVETPSSDNGTCYPGDFI
DYEELREQLSSVSSFERFEIFPKTSSWPNHDSNKGVTAACPHAGAKSF
YKNLIWLVKKGNSYPKLSKSYINDKGKEVLVLWGIHHPSTSADQQSLY
QNADTYVFVGSSRYSKKFKPEIAIRPKVRDQEGRMNYYWTLVEPGDKI
TFEATGNLVVPRYAFAMERNAGSGIIISDTPVHDCNTTCQTPKGAINT
SLPFQNIHPITIGKCPKYVKSTKLRLATGLRNIPSIQSRGLFGAIAGF
IEGGWTGMVDGWYGYHHQNEQGSGYAADLKSTQNAIDEITNKVNSVIE
KMNTQFTAVGKEFNHLEKRIENLNKKVDDGFLDIWTYNAELLVLLENE
RTLDYHDSNVKNLYEKVRSQLKNNAKEIGNGCFEFYHKCDNTCMESVK
NGTYDYPKYSEEAKLNREEIDGVKLESTRIYQIASTTEPATPTTPVTT
PTTTDDLDAVRIKVDTVNAKPGDTVNIPVRFSGIPSKGIANCDFVYSY
DPNVLEIIEIKPGELIVDPNPTKSFDTAVYPDRKMIVFLFAEDSGTGA
YAITKDGVFATIVAKVKEGAPNGLSVIKFVEVGGFANNDLVEQKTQFF
DGGVNVGDTHHHHHH

Influenza: Mam-cetHS-puro[SLAML-6xHis-Cohesin-F1uHAb-1] C2297

(SEQ ID NO: 54)
DITSHHHHHHDDLDAVRIKVDTVNAKPGDTVRIPVRFSGIPSKGIAN
CDFVYSYDPNVLEIIEIEPGDIIVDPNPDKSFDTAVYPDRKIIVFLF
AEDSGTGAYAITKDGVFATIVAKVKEGAPNGLSVIKFVEVGGFANND
LVEQKTQFFDGGVNVGDTTEPATPTTPVTTPTTTDDLDAASDRICTG
ITSSNSPHVVKTATQGEVNVTGVIPLTTTPTKSHFANLKGTETRGKL
CPKCLNCTDLDVALGRPKCTGKIPSARVSILHEVRPVTSGCFPIMHD
RTKIRQLPNLLRGYEHIRLSTHNVINAENAPGGPYKIGTSGSCPNIT
NGNGFFATMAWAVPKNDKNKTATNPLTIEVPYICTEGEDQITVWGFH
SDDETQMAKLYGDSKPQKFTSSANGVTTHYVSQIGGFPNQTEDGGLP
QSGRIVVDYMVQKSGKTGTITYQRGILLPQKVWCASGRSKVIKGSLP
LIGEADCLHEKYGGLNKSKPYYTGEHAKAIGNCPIWVKTPLKLANGT
K

Influenza: Mam-cetHS-puro[SLAML-6xHis-Cohesin-FluM2-1s] C1734

(SEQ ID NO: 55)
GLDITSHHHHHHDDLDAVRIKVDTVNAKPGDTVRIPVRFSGIPSKG
IANCDFVYSYDPNVLEIIEIEPGDIIVDPNPDKSFDTAVYPDRKII
VFLFAEDSGTGAYAITKDGVFATIVAKVKEGAPNGLSVIKFVEVGG
FANNDLVEQKTQFFDGGVNVGDTTEPATPTTPVTTPTTTDDLDAAS
LLTEVETPTRSEWECRCSDSSDPAS

Influenza: Mam-cetHS-puro[SLAML-6xHis-Cohesin-M2e] C1977

(SEQ ID NO: 56)
GLDITSHHHHHHDDLDAVRIKVDTVNAKPGDTVRIPVRFSGIPSKGI
ANCDFVYSYDPNVLEIIEIEPGDIIVDPNPDKSFDTAVYPDRKIIVF
LFAEDSGTGAYAITKDGVFATIVAKVKEGAPNGLSVIKFVEVGGFAN
NDLVEQKTQFFDGGVNVGDTTEPATPTTPVTTPTTTDDLDAASLLTE
VETPIRNEWGCRCNGSSDPAS

Influenza: Mam-cetHS-puro[SLAML-Cohesin-F1uHA1-1-6xHis] C489

(SEQ ID NO: 57)
MDPKGSLSWRILLFLSLAFELSYGLDDLDAVRIKVDTVNAKPGDTV
RIPVRFSGIPSKGIANCDFVYSYDPNVLEIIEIEPGDIIVDPNPDK
SFDTAVYPDRKIIVFLFAEDSGTGAYAITKDGVFATIVAKVKEGAP
NGLSVIKFVEVGGFANNDLVEQKTQFFDGGVNVGDTTEPATPTTPV
TTPTTTDDLDALDDTICIGYHANNSTDTVDTVLEKNVTVTHSVNLL
EDSHNGKLCRLKGIAPLQLGKCNIAGWLLGNPECDPLLPVRSWSYI
VETPNSENGICYPGDFIDYEELREQLSSVSSFERFEIFPKESSWPN
HNTNGVTAACSHEGKSSFYRNLLWLTEKEGSYPKLKNSYVNKKGKE
VLVLWGIHHPPNSKEQQNLYQNENAYVSVVTSNYNRRFTPEIAERP
KVRDQAGRMNYYWTLLKPGDTIIFEANGNLIAPMYAFALSRGFGSG
IITSNASMHECNTKCQTPLGAINSSLPYQNIHPVTIGECPKYVRSA
KLRMVHHHHHH

Influenza: Mam-cetHS-puro[SLAML-Cohesin-F1uHA5-0-6xHis] C543

(SEQ ID NO: 58)
LDDLDAVRIKVDTVNAKPGDTVRIPVRFSGIPSKGIANCDFVYSYDP
NVLEIIEIEPGDIIVDPNPDKSFDTAVYPDRKIIVFLFAEDSGTGAY
AITKDGVFATIVAKVKEGAPNGLSVIKFVEVGGFANNDLVEQKTQFF
DGGVNVGDTTEPATPTTPVTTPTTTDDLDALEDQICIGYHANNSTEQ
VDTIMEKNVTVTHAQDILEKKHNGKLCDLDGVKPLILRDCSVAGWLL
GNPMCDEFINVPEWSYIVEKANPVNDLCYPGDFNDYEELKHLLSRIN
HFEKIQIIPKSSWSSHEASLGVSSACPYQGKSSFFRNVVWLIKKNST
YPTIKRSYNNTNQEDLLVLWGIHHPNDAAEQTKLYQNPTTYISVGTS
TLNQRLVPRIATRSKVNGQSGRMEFFWTILKPNDAINFESNGNFIAP
EYAYKIVKKGDSTIMKSELEYGNCNTKCQTPMGAINSSMPFHNIHPL
TIGECPKYVKSNRLVLATGLRNSPQRERRRKKRGLFGAIAGFIEGGW
QGMVDGWYGYHHSNEQGSGYAADKESTQKAIDGVTNKVNSIIDKMNT
QFEAVGREFNNLERRIENLNKKMEDGFLDVWTYNAELLVLMENERTL
DFHDSNVKNLYDKVRLQLRDNAKELGNGCFEFYHKCDNECMESVRNG
TYDYPQYSEEARLKREEIHHHHHH

Influenza: Mam-cetHS-puro[SLAML-Cohesin-F1uHA5-1-6xHis] C490

(SEQ ID NO: 59)
MDPKGSLSWRILLFLSLAFELSYGLDDLDAVRIKVDTVNAKPGDTVR
IPVRFSGIPSKGIANCDFVYSYDPNVLEIIEIEPGDIIVDPNPDKSF
DTAVYPDRKIIVFLFAEDSGTGAYAITKDGVFATIVAKVKEGAPNGL
SVIKFVEVGGFANNDLVEQKTQFFDGGVNVGDTTEPATPTTPVTTPT
TTDDLDALEDQICIGYHANNSTEQVDTIMEKNVTVTHAQDILEKKHN
GKLCDLDGVKPLILRDCSVAGWLLGNPMCDEFINVPEWSYIVEKANP
VNDLCYPGDFNDYEELKHLLSRINHFEKIQIIPKSSWSSHEASLGVS
SACPYQGKSSFFRNVVWLIKKNSTYPTIKRSYNNTNQEDLLVLWGIH
HPNDAAEQTKLYQNPTTYISVGTSTLNQRLVPRIATRSKVNGQSGRM
EFFWTILKPNDAINFESNGNFIAPEYAYKIVKKGDSTIMKSELEYGN
CNTKCQTPMGAINSSMPFHNIHPLTIGECPKYVKSNRLVLAHHHHHH

SIV: Mam-cetHS-puro[6xHis-Cohesin-ViralgagSIV-p17] C1274

(SEQ ID NO: 60)
LDITSHHHHHHDDLDAVRIKVDTVNAKPGDTVRIPVRFSGIPSKGIA
NCDFVYSYDPNVLEIIEIEPGDIIVDPNPDKSFDTAVYPDRKIIVFL
FAEDSGTGAYAITKDGVFATIVAKVKEGAPNGLSVIKFVEVGGFANN
DLVEQKTQFFDGGVNVGDTTEPATPTTPVTTPTTTDDLDAASMRVRN
SVLSGKKADELEKIRLRPNGKKKYMLKHVVWAANELDRFGLAESLLE
NKEGCQKILSVLAPLVPTGSENLKSLYNTVCVIWCIHAEEKVKHTEE
AAS

SIV: Mam-cetHS-puro[6xHis-Cohesin-ViralgagSIV-p24] C1273

(SEQ ID NO: 61)
LDITSHHHHHHDDLDAVRIKVDTVNAKPGDTVRIPVRFSGIPSKGIA
NCDFVYSYDPNVLEIIEIEPGDIIVDPNPDKSFDTAVYPDRKIIVFL
FAEDSGTGAYAITKDGVFATIVAKVKEGAPNGLSVIKEVEVGGFANN
DLVEQKTQFFDGGVNVGDTTEPATPTTPVTTPTTTDDLDAASKTSRP
TAPSSGRGGNYPVQQIGGNYVHLPLSPRTLNAWVKLIEEKKFGAEVV
PGFQALSEGCTPYDINQMLNCVGDHQAAMQIIRDIINEEAADWDLQH
PQPAPQQGQLREPSGSDIAGTTSSVDEQIQWMYRQQNPIPVGNIYRR
WIQLGLQKCVRMYNPTNILDVKQGPKEPFQSYVDRFYKSLRAEQTDA
AVKNWMTQTLLIQNANPDCKLVLKGLGVNPTLEEMLTACQGVGAS

SIV: Mam-cetHS-puro[6xHis-Cohesin-ViralnefSIV] C1275

(SEQ ID NO: 62)
LDITSHHHHHHDDLDAVRIKVDTVNAKPGDTVRIPVRFSGIPSKGIA
NCDFVYSYDPNVLEIIEIEPGDIIVDPNPDKSFDTAVYPDRKIIVFL
FAEDSGTGAYAITKDGVFATIVAKVKEGAPNGLSVIKFVEVGGFANN
DLVEQKTQFFDGGVNVGDTTEPATPTTPVTTPTTTDDLDAASLGEVE
DGYSQSPGGLDKGLSSLSCEGQKYNQGQYMNTPWRNPAEEREKLAYR
KQNMDDIDEEDDDLVGVSVRPKVPLRTMSYKLAIDMSHFIKEKGGLE
GIYYSARRHRILDIYLEKEEGIIPDWQDYTSGPGIRYPKTFGWLWKL
VPVNVSDEAQEDEEHYLMHPAQTSQWDDPWGEVLAWKFDPTLAYTYE
AYVRYPEEFGSKAS

SIV: Mam-cetHS-puro[SLAML-6xHis-Cohesin-ViralenvSIV-gp41-6xHis] C1193

(SEQ ID NO: 63)
LDITSHHHHHHDDLDAVRIKVDTVNAKPGDTVRIPVRFSGIPSKGIA
NCDFVYSYDPNVLEIIEIEPGDIIVDPNPDKSFDTAVYPDRKIIVFL
FAEDSGTGAYAITKDGVFATIVAKVKEGAPNGLSVIKEVEVGGFANN
DLVEQKTQFFDGGVNVGDTTEPATPTTPVTTPTTTDDLDAASAQSRT
LLAGIVQQQQQLLDVVKRQQELLRLTVWGTKNLQTRVTAIEKYLKDQ
AQLNAWGCAFRQVCHTTVPWPNASLTPKWNNETWQEWERKVDFLEEN
ITALLEEAQIQQEKNMYELQKLNSHHHHHHAS

Tuberculosis: Ecoli-pET28[6xHis-Cohesin-m.tbCFP10] C1254

(SEQ ID NO: 64)
MGSSHHHHHHSSGLVPRGSHMASMDLDAVRIKVDTVNAKPGDTVNIP
VRFSGIPSKGIANCDFVYSYDPNVLEIIEIKPGELIVDPNPTKSFDT
AVYPDRKMIVFLFAEDSGTGAYAITKDGVFATIVAKVKEGAPNGLSV
IKFVEVGGFANNDLVEQKTQFFDGGVNVGDTTEPATPTTPVTTPTTT
DDLDAASMAEMKTDAATLAQEAGNFERISGDLKTQIDQVESTAGSLQ
GQWRGAAGTAAQAAVVRFQEAANKQKQELDEISTNIRQAGVQYSRAD
EEQQQALSSQMGF

Tuberculosis: Ecoli-pET28[6xHis-Cohesin-m.tbRv0125] C1334

(SEQ ID NO: 65)
MGSSHHHHHHSSGLVPRGSHMASMDLDAVRIKVDTVNAKPGDTVNIP
VRFSGIPSKGIANCDFVYSYDPNVLEIIEIKPGELIVDPNPTKSFDT
AVYPDRKMIVFLFAEDSGTGAYAITKDGVFATIVAKVKEGAPNGLSV
IKFVEVGGFANNDLVEQKTQFFDGGVNVGDTTEPATPTTPVTTPTTT
DDLDAASMSNSRRRSLRWSWLLSVLAAVGLGLATAPAQAAPPALSQD
RFADFPALPLDPSAMVAQVGPQVVNINTKLGYNNAVGAGTGIVIDPN
GVVLTNNHVIAGATDINAFSVGSGQTYGVDVVGYDRTQDVAVLQLRG
AGGLPSAAIGGGVAVGEPVVAMGNSGGQGGTPRAVPGRVVALGQTVQ
ASDSLTGAEETLNGLIQFDAAIQPGDSGGPVVNGLGQVVGMNTAASD
NFQLSQGGQGFAIPIGQAMAIAGQIRSGGGSPTVHIGPTAFLGLGVV
DNNGNGARVQRVVGSAPAASLGISTGDVITAVDGAPINSATAMADAL
NGHHPGDVISVTWQTKSGGTRTGNVTLAEGPPA

Tuberculosis: Ecoli-pET28[6xHis-Cohesin-m.tbRv0570] C1500

(SEQ ID NO: 66)
MGSSHHHHHHSSGLVPRGSHMASMDLDAVRIKVDTVNAKPGDTVNI
PVRFSGIPSKGIANCDFVYSYDPNVLEIIEIKPGELIVDPNPTKSF
DTAVYPDRKMIVFLFAEDSGTGAYAITKDGVFATIVAKVKEGAPNG
LSVIKFVEVGGFANNDLVEQKTQFFDGGVNVGDTTEPATPTTPVTT
PTTTDDLDAASVGVSWPAKVRRRDGTLVPFDIARIEAAVTRAAREV
ACDDPDMPGTVAKAVADALGRGIAPVEDIQDCVEARLGEAGLDDVA
RVYIIYRQRRAELRTAKALLGVRDELKLSLAAVTVLRERYLLHDEQ
GRPAESTGELMDRSARCVAAAEDQYEPGSSRRWAERFATLLRNLEF
LPNSPTLMNSGTDLGLLAGCFVLPIEDSLOSIFATLGOAAELQRAG
GGTGYAFSHLRPAGDRVASTGGTASGPVSFLRLYDSAAGVVSMGGR
RRGACMAVLDVSHPDICDFVTAKAESPSELPHFNLSVGVTDAFLRA
VERNGLHRLVNPRTGKIVARMPAAELFDAICKAAHAGGDPGLVFLD
TINRANPVPGRGRIEATNPCGEVPLLPYESCNLGSINLARMLADGR
VDWDRLEEVAGVAVRFLDDVIDVSRYPFPELGEAARATRKIGLGVM
GLAELLAALGIPYDSEEAVRLATRLMRRIQQAAHTASRRLAEERGA
FPAFTDSRFARSGPRRNAQVTSVAPTGTISLIAGTTAGIEPMFAIA
FTRAIVGRHLLEVNPCFDRLARDRGFYRDELIAEIAORGGVRGYPR
LPAEVRAAFPTAAEIAPOWHLRMQAAVORHVEAAVSKTVNLPATAT
VDDVRAIYVAAWKAKVKGITVYRYGSREGQVLSYAAPKPLLAQADT
EFSGGCAGRSCEF

Tuberculosis: Ecoli-pET28[6xHis-Cohesin-m.tbRv0577] C1335

(SEQ ID NO: 67)
MNRLTSSLLLLIVPAYVLSQVTLKESGPGILQPSQTLSLTCSFSGFS
LSTSGMGLSWIRQPSGKGLEWLAHIYWDDDKRYNPSLKSRLTISKDT
SSNQVFLKITIVDTADAATYYCARSSHYYGYGYGGYFDVWGAGTTVT
VSSAKTKGPSVFPLAPCSRSTSESTAALGCLVKDYFPEPVTVSWNSG
ALTSGVHTFPAVLQSSGLYSLSSVVTVPSSSLGTKTYTCNVDHKPSN
TKVDKRVESKYGPPCPPCPAPEFEGGPSVFLFPPKPKDTLMISRTPE
VTCVVVDVSQEDPEVQFNWYVDGVEVHNAKTKPREEQFNSTYRVVSV
LTVLHQDWLNGKEYKCKVSNKGLPSSIEKTISKAKGQPREPQVYTLP
PSQEEMTKNQVSLTCLVKGFYPSDIAVEWESNGQPENNYKTTPPVLD
SDGSFFLYSRLTVDKSRWQEGNVFSCSVMHEALHNHYTQKSLSLSLG
KASQTPTNTISVTPTNNSTPTNNSNPKPNPASMPKRSEYRQGTPNWV
DLQTTDQSAAKKFYTSLFGWGYDDNPVPGGGGVYSMATLNGEAVAAI
APMPPGAPEGMPPIWNTYIAVDDVDAVVDKVVPGGGQVMMPAFDIGD
AGRMSFITDPTGAAVGLWQANRHIGATLVNETGTLIWNELLTDKPDL
ALAFYEAVVGLTHSSMEIAAGQNYRVLKAGDAEVGGCMEPPMPGVPN
HWHVYFAVDDADATAAKAAAAGGQVIAEPADIPSVGRFAVLSDPQGA
IFSVLKPAPQQ

Tuberculosis: Ecoli-pET28[6xHis-Cohesin-m.tbRv1626] C1256

(SEQ ID NO: 68)
MGSSHHHHHHSSGLVPRGSHMASMDLDAVRIKVDTVNAKPGDTVNIP
VRFSGIPSKGIANCDFVYSYDPNVLEIIEIKPGELIVDPNPTKSFDT
AVYPDRKMIVFLFAEDSGTGAYAITKDGVFATIVAKVKEGAPNGLSV
IKFVEVGGFANNDLVEQKTQFFDGGVNVGDTTEPATPTTPVTTPTTT
DDLDAASMTGPTTDADAAVPRRVLIAEDEALIRMDLAEMLREEGYEI
VGEAGDGQEAVELAELHKPDLVIMDVKMPRRDGIDAASEIASKRIAP
IVVLTAFSQRDLVERARDAGAMAYLVKPFSISDLIPAIELAVSRFRE
ITALEGEVATLSERLETRKLVERAKGLLQTKHGMTEPDAFKWIQRAA
MDRRTTMKRVAEVVLETLGTPKDT

Tuberculosis: Ecoli-pET28[6xHis-Cohesin-m.tbRv2875] C1310

(SEQ ID NO: 69)
VFAPTNAAFSKLPASTIDELKTNSSLLTSILTYHVVAGQTSPANVVG
TRQTLQGASVTVTGQGNSLKVGNADVVCGGVSTANATVYMIDSVLMP
PA

Tuberculosis: Ecoli-pET28[6xHis-Cohesin-m.tbRv3044] C1312

(SEQ ID NO: 70)
MGSSHHHHHHSSGLVPRGSHMASMDLDAVRIKVDTVNAKPGDTVNIP
VRFSGIPSKGIANCDFVYSYDPNVLEIIEIKPGELIVDPNPTKSFDT
AVYPDRKMIVFLFAEDSGTGAYAITKDGVFATIVAKVKEGAPNGLSV
IKFVEVGGFANNDLVEQKTQFFDGGVNVGDTTEPATPTTPVTTPTTT
DDLDAASMRSTVAVAVAAAVIAASSGCGSDQPAHKASQSMITPTTQI
AGAGVLGNDRKPDESCARAAAAADPGPPTRPAHNAAGVSPEMVQVPA
EAQRIVVLSGDQLDALCALGLQSRIVAAALPNSSSSQPSYLGTTVHD
LPGVGTRSAPDLRAIAAAHPDLILGSQGLTPQLYPQLAAIAPTVFTA
APGADWENNLRGVGAATARIAAVDALITGFAEHATQVGTKHDATHFQ
ASIVQLTANTMRVYGANNFPASVLSAVGVDRPPSORFTDKAYIEIGT
TAADLAKSPDFSAADADIVYLSCASEAAAERAAVILDSDPWRKLSAN
RDNRVFVVNDQVWQTGEGMVAARGIVDDLRWVDAPIN

Tuberculosis: Ecoli-pET28[6xHis-Cohesin-m.tbRv3478] C1308

(SEQ ID NO: 71)
MGSSHHHHHHSSGLVPRGSHMASMDLDAVRIKVDTVNAKPGDTVNI
PVRFSGIPSKGIANCDFVYSYDPNVLEIIEIKPGELIVDPNPTKSF
DTAVYPDRKMIVFLFAEDSGTGAYAITKDGVFATIVAKVKEGAPNG
LSVIKFVEVGGFANNDLVEQKTQFFDGGVNVGDTTEPATPTTPVTT
PTTTDDLDAASVVDFGALPPEINSARMYAGPGSASLVAAAKMWDSV
ASDLFSAASAFQSVVWGLTVGSWIGSSAGLMAAAASPYVAWMSVTA
GQAQLTAAQVRVAAAAYETAYRLTVPPPVIAENRTELMTLTATNLL
GQNTPAIEANQAAYSQMWGQDAEAMYGYAATAATATEALLPFEDAP
LITNPGGLLEQAVAVEEAIDTAAANQLMNNVPQALQQLAQPAQGVV
PSSKLGGLWTAVSPHLSPLSNVSSIANNHMSMMGTGVSMTNTLHSM
LKGLAPAAAQAVETAAENGVWAMSSLGSQLGSSLGSSGLGAGVAAN
LGRAASVGSLSVPPAWAAANQAVTPAARALPLTSLTSAAQTAPGHM
LGGLPLGHSVNAGSGINNALRVPARAYAIPRTPAAG

Tuberculosis: Ecoli-pET28[6xHis-CthermoCohesin-m.tbAg85BDe141] C2200

(SEQ ID NO: 72)
MGSSHHHHHHSSGLVPRGSHMASMDLDAVRIKVDTVNAKPGDTVNI
PVRFSGIPSKGIANCDFVYSYDPNVLEIIEIKPGELIVDPNPTKSF
DTAVYPDRKMIVFLFAEDSGTGAYAITKDGVFATIVAKVKEGAPNG
LSVIKFVEVGGFANNDLVEQKTQFFDGGVNVGDTTEPATPTTPVTT
PTTTDDLDAASSRPGLPVEYLQVPSPSMGRDIKVQFQSGGNNSPAV
YLLDGLRAQDDYNGWDINTPAFEWYYQSGLSIVMPVGGQSSFYSDW
YSPACGKAGCQTYKWETFLTSELPQWLSANRAVKPTGSAAIGLSMA
GSSAMILAAYHPQQFIYAGSLSALLDPSQGMGPSLIGLAMGDAGGY
KAADMWGPSSDPAWERNDPTQQIPKLVANNTRLWVYCGNGTPNELG
GANIPAEFLENFVRSSNLKFQDAYNAAGGHNAVFNFPPNGTHSWEY
WGAQLNAMKGDLQSSLGAG

Tuberculosis: Ecoli-pET28[6xHis-CthermoCohesin-m.tbMtb72f] C2236

(SEQ ID NO: 73)
MGSSHHHHHHSSGLVPRGSHMASMDLDAVRIKVDTVNAKPGDTVNIP
VRFSGIPSKGIANCDFVYSYDPNVLEIIEIKPGELIVDPNPTKSFDT
AVYPDRKMIVFLFAEDSGTGAYAITKDGVFATIVAKVKEGAPNGLSV
IKFVEVGGFANNDLVEQKTQFFDGGVNVGDTTEPATPTTPVTTPTTT
DDLDAASTAASDNFQLSQGGQGFAIPIGQAMAIAGQIRSGGGSPTVH
IGPTAFLGLGVVDNNGNGARVQRVVGSAPAASLGISTGDVITAVDGA
PINSATAMADALNGHHPGDVISVTWQTKSGGTRTGNVTLAEGPPAEF
MVDFGALPPEINSARMYAGPGSASLVAAAQMWDSVASDLFSAASAFQ
SVVWGLTVGSWIGSSAGLMVAAASPYVAWMSVTAGQAELTAAQVRVA
AAAYETAYGLTVPPPVIAENRAELMILIATNLLGQNTPAIAVNEAEY
GEMWAQDAAAMFGYAAATATATATLLPFEEAPEMTSAGGLLEQAAAV
EEASDTAAANQLMNNVPQALQQLAQPTQGTTPSSKLGGLWKTVSPHR
SPISNMVSMANNHMSMTNSGVSMTNTLSSMLKGFAPAAAAQAVQTAA
QNGVRAMSSLGSSLGSSGLGGGVAANLGRAASVGSLSVPQAWAAANQ
AVTPAARALPLTSLTSAAERGPGQMLGGLPVGQMGARAGGGLSGVLR
VPPRPYVMPHSPAAGDIAPPALSQDRFADFPALPLDPSAMVAQVGPQ
VVNINTKLGYNNAVGAGTGIVIDPNGVVLTNNHVIAGATDINAFSVG
SGQTYGVDVVGYDRTQDVAVLQLRGAGGLPSAAIGGGVAVGEPVVAM
GNSGGQGGTPRAVPGRVVALGQTVQASDSLTGAEETLNGLIQFDAAI
QPGDSGGPVVNGLGQVVGMNTAAS

Tuberculosis: Ecoli-pET28[6xHis-CthermoCohesin-m.tbRv0288] C2202

(SEQ ID NO: 74)
MGSSHHHHHHSSGLVPRGSHMASMDLDAVRIKVDTVNAKPGDTVNIP
VRFSGIPSKGIANCDFVYSYDPNVLEIIEIKPGELIVDPNPTKSFDT
AVYPDRKMIVFLFAEDSGTGAYAITKDGVFATIVAKVKEGAPNGLSV
IKFVEVGGFANNDLVEQKTQFFDGGVNVGDTTEPATPTTPVTTPTTT
DDLDAASMSQIMYNYPAMLGHAGDMAGYAGTLQSLGAEIAVEQAALQ
SAWQGDTGITYQAWQAQWNQAMEDLVRAYHAMSSTHEANTMAMMARD
TAEAAKWGG

Tuberculosis: Ecoli-pET28[6xHis-CthermoCohesin-m.tbRv0475] C2199

(SEQ ID NO: 75)
MGSSHHHHHHSSGLVPRGSHMASMDLDAVRIKVDTVNAKPGDTVNIP
VRFSGIPSKGIANCDFVYSYDPNVLEIIEIKPGELIVDPNPTKSFDT
AVYPDRKMIVFLFAEDSGTGAYAITKDGVFATIVAKVKEGAPNGLSV
IKFVEVGGFANNDLVEQKTQFFDGGVNVGDTTEPATPTTPVTTPTTT
DDLDAASCSCMAENSNIDDIKAPLLAALGAADLALATVNELITNLRE
RAEETRTDTRSRVEESRARLTKLQEDLPEQLTELREKFTAEELRKAA
EGYLEAATSRYNELVERGEAALERLRSQQSFEEVSARAEGYVDQAVE
LTQEALGTVASQTRAVGERAAKLVGIELPKKAAPAKKAAPAKKAAPA
KKAAAKKAPAKKAAAKKVTQKCSC

Tuberculosis: Ecoli-pET28[6xHis-CthermoCohesin-m.tbRv1980] C2197

(SEQ ID NO: 76)
MGSSHHHHHHSSGLVPRGSHMASMDLDAVRIKVDTVNAKPGDTVNI
PVRFSGIPSKGIANCDFVYSYDPNVLEIIEIKPGELIVDPNPTKSF
DTAVYPDRKMIVFLFAEDSGTGAYAITKDGVFATIVAKVKEGAPNG
LSVIKFVEVGGFANNDLVEQKTQFFDGGVNVGDTTEPATPTTPVTT
PTTTDDLDAASAAPKTYCEELKGTDTGQACQIQMSDPAYNINISLP
SYYPDQKSLENYIAQTRDKFLSAATSSTPREAPYELNITSATYQSA
IPPRGTQAVVLKVYQNAGGTHPTTTYKAFDWDQAYRKPITYDTLWQ
ADTDPLPVVFPIVQGELSKQTGQQVSIAPNAGLDPVNYQNFAVTND
GVIFFFNPGELLPEAAGPTQVLVPRSAIDSMLAEF

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

U.S. Patent Application Publication No. 2010/0062451: Bead-Ligand-Nascent Protein Complexes.

U.S. Patent Application Publication No. 2005/0106700: Method of Purifying Recombinant Fused Protein and Method of Producing Protein Using the Same.

U.S. Patent Application Publication No. 2011/0151538: Affinity Purification by Cohesin-Dockerin Interaction.

What is claimed is:

1. A method for detecting, isolating, or purifying one or more analytes in a sample, in a matrix, from a mixture, or any combinations thereof comprising:
obtaining a solid substrate comprising a first member of a cohesin-dockerin binding pair, wherein the first member is attached to a protein, an antibody, an antigen, a peptide, a toxin, a cytokine, an enzyme, a structural protein, an extracellular matrix protein, a cell, or fragments thereof, wherein the solid substrate is selected from the group consisting of a bead, a cell, an extracellular matrix, a fibrous matrix, a container, a chip, an affinity column, and any combinations thereof
providing a second member of the cohesin-dockerin binding pair, wherein the second member is present in the sample, the matrix, the mixture or any combinations thereof, wherein the second member is capable of binding to one or more analytes to be detected, isolated, or purified from the sample, the matrix, the mixture or any combinations thereof
contacting the second member of the cohesin-dockerin binding pair with the sample, the matrix or the mixture suspected of having the analyte; and
forming a complex on the substrate comprising the first and the second members of the cohesin-dockerin binding pairs and the analyte, wherein the presence of the analyte is detected.

2. The method of claim 1, further comprising:
adding a detection reagent to the complex for determining presence or absence of the analyte, wherein the detection reagent comprises a secondary antibody, a radiolabel, a flurophore, a colorimetric reagent, or any combinations thereof

3. The method of claim 2, wherein the detection agent is part of the second member.

4. The method of claim 1, wherein the analyte comprises a nucleic acid (natural and unnatural, aptamers), carbohydrates, polysaccharides, peptides, proteins or peptoids (natural and unnatural), minerals, vitamins or lipids.

5. The method of claim 1, wherein the first member comprises a dockerin domain and the second member comprises a cohesin domain bound to the analyte.

6. The method of claim 1, wherein the first member comprises a cohesin domain and the second member comprises a dockerin domain bound to the analyte.

7. The method of claim 1, wherein the second member comprises a cohesin or a dockerin domain and the analyte forms a fusion protein with the cohesin or dockerin domain.

8. The method of claim 1, wherein the method is defined further as carried out in a container that comprises a beaker, a flask, a cylinder, a test tube, a centrifugation tube, a petri dish, a culture dish, a multi-well plate or a chip.

9. The method of claim 1, wherein the substrate comprises one or more sets of beads, wherein the beads comprise nanospheres, nanoparticles, microspheres, microparticles, nanobeads, microbeads, beads, polystyrene beads, latex particles, latex beads, fluorescent beads, fluorescent particles, colored particles, or colored beads.

10. The method of claim 1, wherein the one or more sets of beads comprise polymeric beads, wherein the polymers are selected from the group consisting of carbohydrate-based polymers, polyaliphatic alcohols, poly(vinyl) polymers, polyacrylic acids, polyorganic acids, polyamino acids, co-polymers, block co-polymers, tertpolymers, polyethers, naturally occurring polymers, polyimids, surfactants, polyesters, branched polymers, cyclo-polymers, polyaldehydes and mixtures thereof, brominated polystyrene, polyacrylic acid, polyacrylonitrile, polyamide, polyacrylamide, polyacrolein, polybutadiene, polycaprolactone, polyester, polyethylene, polyethylene terephthalate, polydimethylsiloxane, polyisoprene, polyurethane, polyvinylacetate, polyvinylchloride, polyvinylpyridine, polyvinylbenzylchloride, polyvinyltoluene, polyvinylidene chloride, polydivinylbenzene, polymethylmethacrylate, polylactide, polyglycolide, poly(lactide-co-glycolide), polyanhydride, polyorthoester, polyphosphazene, polyphosophaze, poly-(styrene-co-vinylbenzyl chloride-co-acrylic acid) (85:10:5 molar ratio), poly(styrene-co-acrylic acid) (99:1 molar ratio), poly(styrene-co-methacrylic acid) (90:10 molar ratio), poly(styrene-co-acrylic acid-co-m&p-divinylbenzene) (89:10:1 molar ratio), poly-(styrene-co-2-carboxyethyl acrylate) (90:10 molar ratio), poly(methyl methacrylate-co-acrylic acid) (70:30 molar ratio) and poly(styrene-co-butyl acrylate-co-methacrylic acid)(45:45:10 weight ratio) synthetic polymers polystyrene, polyacrylamide, polyacrylate, latex, and any combinations or modifications thereof

11. The method of claim 1, wherein the one or more analytes comprise one or more antigen, antibody, autoantibody, peptide, protein, nucleic acid sequence, or enzyme or any combination thereof, wherein the antigens comprise one or more bacterial, viral, fungal, mycoplasmal, rickettsial, chlamydial, or protozoal antigens, or any combination thereof.

12. The method of claim 1, wherein the dockerin may be a substituted dockerin, a truncated dockerin, a modified dockerin, or any combinations thereof.

13. The method of claim 1, wherein the cohesin may be a substituted cohesin, a truncated cohesin, a modified cohesin, or any combinations thereof.

14. The method of claim 1, wherein the cohesin is derived or isolated from Clostridium thermocellum, C. cellulolyticum, C. cellulovorans, C. papyrosolvens, and Clostridium cellobioparum, C. papyrosolvens, C. josui, Acetivibrio cellulolyticus, Bacteroides cellulosolvens, R. flavefaciens, Ruminococcus albus, Archaeoglobus fulgidus protein, or cellulosomal cohesin domain.

15. A multiplex bead based method for detecting, isolating, or purifying one or more analytes in a sample, in a matrix, from a mixture, or any combinations thereof comprising:
providing one, a plurality, or a set of beads comprising nanospheres, nanoparticles, microspheres, microparticles, nanobeads, microbeads, beads, polystyrene beads, latex particles, latex beads, fluorescent beads, fluorescent particles, colored particles, or colored beads;
attaching a first member of a cohesin-dockerin binding pair to the beads, wherein the first member is attached to a protein, an antibody, an antigen, a peptide, a toxin, a cytokine, an enzyme, a structural protein, an extracellular matrix protein, a cell, or a fragment thereof;
providing at least one second member of the cohesin-dockerin binding pair attached to the second member is attached to the one or more analytes to be detected, isolated, or purified from the sample, the matrix, the mixture or any combinations thereof;
contacting the first member of the cohesin-dockerin binding pair with the sample, the matrix or the mixture comprising the second cohesin-dockerin binding pair; and
forming a complex comprising at least one second member of the cohesin-dockerin binding pair to the at least one first member of the cohesin-dockerin binding pair.

16. The method of claim 15, further comprising:
adding a detection reagent to the complex for determining presence or absence of the analyte, wherein the detection reagent comprises a secondary antibody, a radiolabel, a flurophore, a colorimetric reagent, or any combinations thereof;
releasing the second member comprising the analyte from the complex by one or more physical or chemical methods; and
isolating the analyte from a mixture comprising cohesin, dockerin, proteins, antigens, peptides, antibodies, or any combinations thereof

17. The method of claim 16, wherein the detection agent is part of the second member.

18. The method of claim 15, wherein the analyte comprises a nucleic acid (natural and unnatural, aptamers), carbohydrates, polysaccharides, peptides, proteins or peptoids (natural and unnatural), minerals, vitamins or lipids.

19. The method of claim 15, wherein the first member comprises a dockerin domain and the second member comprises a cohesin domain bound to the analyte.

20. The method of claim 15, wherein the first member comprises a cohesin domain and the second member comprises a dockerin domain bound to the analyte.

21. The method of claim 15, wherein the second member comprises a cohesin or a dockerin domain and the analyte forms a fusion protein with the cohesin or dockerin domain.

22. The method of claim 15, wherein the one or more sets of beads comprise polymeric beads, wherein the polymers are selected from the group consisting of carbohydrate-based polymers, polyaliphatic alcohols, poly(vinyl) polymers, polyacrylic acids, polyorganic acids, polyamino acids, co-polymers, block co-polymers, tertpolymers, polyethers, naturally occurring polymers, polyimids, surfactants, polyesters, branched polymers, cyclo-polymers, polyaldehydes and mixtures thereof, brominated polystyrene, polyacrylic acid, polyacrylonitrile, polyamide, polyacrylamide, polyacrolein, polybutadiene, polycaprolactone, polyester, polyethylene, polyethylene terephthalate, polydimethylsiloxane, polyisoprene, polyurethane, polyvinylacetate, polyvinylchloride, polyvinylpyridine, polyvinylbenzylchloride, polyvinyltoluene, polyvinylidene chloride, polydivinylbenzene, polymethylmethacrylate, polylactide, polyglycolide, poly(lactide-co-glycolide), polyanhydride, polyorthoester, polyphosphazene, polyphosophaze,poly-(styrene-co-vinylbenzyl chloride-co-acrylic acid) (85:10:5 molar ratio), poly(styrene-co-acrylic acid) (99:1 molar ratio), poly(styrene-co-methacrylic acid) (90:10 molar ratio), poly(styrene-co-acrylic acid-co-m&p-divinylbenzene) (89:10:1 molar ratio), poly-(styrene-co-2-carboxyethyl acrylate) (90:10 molar ratio), poly(methyl methacrylate-co-acrylic acid) (70:30 molar ratio) and poly(styrene-co-butyl acrylate-co-methacrylic acid)(45:45:10 weight ratio) synthetic polymers polystyrene, polyacrylamide, polyacrylate, latex, and any combinations or modifications thereof

23. The method of claim 15, wherein the one or more analytes comprise one or more antigen, antibody, autoantibody, peptide, protein, nucleic acid sequence, or enzyme or any combination thereof, wherein the antigens comprise one or more bacterial, viral, fungal, mycoplasmal, rickettsial, chlamydial, or protozoal antigens, or any combination thereof.

24. The method of claim 15, wherein the dockerin is selected from a Domain I dockerin, a Domain II dockerin, a Domain III dockerin, a substituted dockerin, a truncated dockerin, a modified dockerin, or any combinations thereof.

25. The method of claim 15, wherein the cohesin may be a Type I cohesin, a Type II cohesin, a Type III cohesin, a substituted cohesin, a truncated cohesin, a modified cohesin, or any combinations thereof

26. An assay system comprising:
a substrate and at least one attached or immobilized dockerin or cohesin binding domain bound to the substrate, and
a dockerin or cohesin binding pair is attached to a protein, an antibody, an antigen, a peptide, a toxin, a cytokine, an enzyme, a structural protein, an extracellular matrix protein, a cell, or a fragment thereof

27. The assay system of claim 26, wherein the substrate comprises one or more beads.

28. The assay system of claim 26, wherein the substrate comprises nanospheres, nanoparticles, microspheres, microparticles, nanobeads, microbeads, beads, polystyrene beads, latex particles, latex beads, fluorescent beads, fluorescent particles, colored particles, or colored beads.

29. The assay system of claim 26, wherein the analyte comprises a nucleic acid (natural and unnatural, aptamers), carbohydrates, polysaccharides, peptides, proteins or peptoids (natural and unnatural), minerals, vitamins or lipids.

30. The assay system of claim 26, wherein the first member comprises a dockerin domain and the second member comprises a cohesin domain bound to the analyte.

31. The assay system of claim 26, wherein the first member comprises a cohesin domain and the second member comprises a dockerin domain bound to the analyte.

32. The assay system of claim 26, wherein the second member comprises a cohesin or a dockerin domain and the analyte forms a fusion protein with the cohesin or dockerin domain.

33. A method for performing an immunoassay on a bead surface for the simultaneous detection of more than one analyte from a sample, a matrix, or a mixture comprising the steps of:
providing one or more beads or sets of beads on a substrate, wherein the substrate is selected from the group consisting of a beaker, a flask, a cylinder, a test tube, a centrifugation tube, a petri dish, a culture dish, and a multi-well plate, or any combinations or modifications thereof;
attaching or immobilizing at least two dockerin binding domain to a surface of the beads, wherein the dockerin binding domain may be attached to a protein, an antibody, an antigen, a peptide, a toxin, a cytokine, an enzyme, a structural protein, an extracellular matrix protein, a cell, or fragments thereof;
contacting the beads with the attached or immobilized dockerin binding domains with the sample, the matrix or the mixture comprising the multiple analytes of interest, wherein the multiple analytes are attached to one or more cohesin fusion proteins;
forming more than one complexes comprising the multiple cohesin fusion protein attached analyte with the multiple dockerin binding domains on the surface of the one or more beads;
adding multiple detecting reagents or labels to the beads, wherein each of the detecting regent or label is specific and binds to the multiple analytes suspected of being present in the sample, matrix, or the mixture, and the detection reagents comprise a secondary antibody, a radiolabel, a flurophore, a colorimetric reagent, or any combinations thereof; and
detecting the presence or absence of the multiple analytes be reading, monitoring, or measuring a signal emitted by the bound detection reagent or label and the analyte.

34. The method of claim 33, further comprising the optional steps of:
performing one or more wash steps with a suitable buffer or water at one or in between different steps of the immunoassay;
generating a calibration curve or a standard curve for determination of a concentration or an amount of the multiple analytes in the sample, the matrix, or the mixture by performing the immunoassay using one or more pure analytes or standards;
releasing the cohesin fusion protein attached to the analyte from the complex by one or more physical or chemical methods; and
isolating the analyte from a mixture comprising cohesin, dockerin, proteins, antigens, peptides, antibodies, or any combinations thereof

35. The method of claim 33, wherein the detection agent is part of the second member.

36. The method of claim 33, wherein the substrate comprises one or more sets of beads, wherein the beads comprise nanospheres, nanoparticles, microspheres, microparticles, nanobeads, microbeads, beads, polystyrene beads, latex particles, latex beads, fluorescent beads, fluorescent particles, colored particles, or colored beads.

37. The method of claim 33, wherein the one or more sets of beads comprise polymeric beads, wherein the polymers are selected from the group consisting of carbohydrate-based polymers, polyaliphatic alcohols, poly(vinyl) polymers, polyacrylic acids, polyorganic acids, polyamino acids, co-polymers, block co-polymers, tertpolymers, polyethers, naturally occurring polymers, polyimide, surfactants, polyesters, branched polymers, cyclo-polymers, polyaldehydes and mixtures thereof, brominated polystyrene, polyacrylic acid, polyacrylonitrile, polyamide, polyacrylamide, polyacrolein, polybutadiene, polycaprolactone, polyester, polyethylene, polyethylene terephthalate, polydimethylsiloxane, polyisoprene, polyurethane, polyvinylacetate, polyvinylchloride, polyvinylpyridine, polyvinylbenzylchloride, polyvinyltoluene, polyvinylidene chloride, polydivinylbenzene, polymethylmethacrylate, polylactide, polyglycolide, poly(lactide-co-glycolide), polyanhydride, polyorthoester, polyphosphazene, polyphosophaze, poly-(styrene-co-vinylbenzyl chloride-co-acrylic acid) (85:10:5 molar ratio), poly(styrene-co-acrylic acid) (99:1 molar ratio), poly(styrene-co-methacrylic acid) (90:10 molar ratio), poly(styrene-co-acrylic acid-co-m&p-divinylbenzene) (89:10:1 molar ratio), poly-(styrene-co-2-carboxyethyl acrylate) (90:10 molar ratio), poly(methyl methacrylate-co-acrylic acid) (70:30 molar ratio) and poly(styrene-co-butyl acrylate-co-methacrylic acid)(45:45:10 weight ratio) synthetic polymers polystyrene, polyacrylamide, polyacrylate, latex, or any combinations or modifications thereof.

38. The method of claim 33, wherein the beads may be free flowing beads or may be attached to the solid substrate.

39. The method of claim 33, wherein the one or more analytes comprise one or more antigen, antibody, autoantibody, peptide, protein, nucleic acid sequence, or enzyme or any combination thereof, wherein the antigens comprise one or more bacterial, viral, fungal, mycoplasmal, rickettsial, chlamydial, or protozoal antigens, or any combination thereof.

40. The method of claim 33, wherein the dockerin may be a substituted dockerin, a truncated dockerin, a modified dockerin, or any combinations thereof.

41. The method of claim 33, wherein the cohesin may be a substituted cohesin, a truncated cohesin, a modified cohesin, or any combinations thereof.

42. The method of claim 33, wherein the cohesin is derived or isolated from Clostridium thermocellum, C. cellulolyticum, C. cellulovorans, C. papyrosolvens, and Clostridium cellobioparum, C. papyrosolvens, C. josui, Acetivibrio cellulolyticus, Bacteroides cellulosolvens, R. flavefaciens, Ruminococcus albus, Archaeoglobus fulgidus protein, or cellulosomal cohesin domain.

43. An affinity purification method utilizing one or more beads comprising the steps of:
providing one or more beads or bead sets on a substrate, wherein the substrate is selected from the group consisting of a beaker, a flask, a cylinder, a test tube, a centrifugation tube, a petri dish, a culture dish, and a multi-well plate, an column or any combinations or modifications thereof;
attaching or immobilizing at least one dockerin binding domain to a surface of the beads, wherein the dockerin binding domain may be attached to a protein, an antibody, an antigen, a peptide, a toxin, a cytokine, an enzyme, a structural protein, an extracellular matrix protein, a cell, or fragments thereof;
contacting the beads by flowing or pumping a sample, a cellular mixture, a fermentation medium, a cell extract, or any combinations thereof comprising one or more analytes to be purified, wherein at least one of the analyte to be purified is couple or attached to a cohesin fusion protein;
binding the analyte comprising attached cohesin fusion protein with the one or more beads comprising at least one dockerin binding domain to form a complex, wherein the binding generates a flow through comprising one or more undesirable materials or materials from which an isolation of the analyte is desired; and
releasing the desired analyte from the complex by one or a combination of physical or chemical methods, wherein the methods comprise a change in ionic strengths, addition of EDTA, removal of Ca²⁺ from the medium, pH, temperature, or any combinations thereof.

44. The method of claim 43, wherein the one or more beads are immobilized to a solid substrate or a column packing material.

45. The method of claim 43, wherein the one or more beads or bead sets are packed in a column.

46. The method of claim 43, wherein the one or more beads are polymeric beads and comprise nanospheres, nanoparticles, microspheres, microparticles, nanobeads, microbeads, beads, polystyrene beads, latex particles, latex beads, fluorescent beads, fluorescent particles, colored particles, or colored beads.

47. The method of claim 43, wherein the analyte to be purified comprises antigens, antibodies, autoantibodies, peptides, proteins, fusion proteins, nucleic acid sequences, and/or enzymes, wherein the antigens comprise one or more bacterial, viral, fungal, mycoplasmal, rickettsial, chlamydial, and/or protozoal antigens.

48. The method of claim 43, wherein the dockerin may be a substituted dockerin, a truncated dockerin, a modified dockerin, or any combinations thereof

49. The method of claim 43, wherein the cohesin may be a substituted cohesin, a truncated cohesin, a modified cohesin, or any combinations thereof.

50. The method of claim 43, wherein the cohesin is derived or isolated from Clostridium thermocellum, C. cellulolyticum, C. cellulovorans, C. papyrosolvens, and Clostridium cellobioparum, C. papyrosolvens, C. josui, Acetivibrio cellulolyticus, Bacteroides cellulosolvens, R. flavefaciens, Ruminococcus albus, Archaeoglobus fulgidus protein, or cellulosomal cohesin domain.


SEQUENCE LISTING (76 sequences)

SEQ ID NO: 1 - protein, 2299 aa, artificial sequence
  synthetic peptid
MQSPRLKRKILSVILAVCYIISSFSIQFAATPQVNIIIGSAQGIPGSTVKVPINLQNVPEIGINNCDFTIKFDSDILDFNSVEAGDIVPLPVASFSSNNSKDIIKFLFSDATQGNMPINENGLFAVISFKIKDNAQKGISNIKVSSYGSFSGMSGKEMQSLSPTFFSGSIDVSDVSTSKLDVKVGNVEGIAGTEVNVPITFENVPDNGINNCNFTLSYDSNALEFLTTEAGNIIPLAIADYSSYRSMEGKIKFLFSDSSQGTRSIKNDGVFANIKFKIKGNAIRDTYRIDLSELGSFSSKQNNNLKSIATQFLSGSVNVKDIESSVSPTTSVHPTPTSVPPTPTKSSPGNKMKIQIGDVKANQGDTVIVPITFNEVPVMGVNNCNFTLAYDKNIMEFISADAGDIVTLPMANYSYNMPSDGLVKFLYNDQAQGAMSIKEDGTFANVKFKIKQSAAFGKYSVGIKAIGSISALSNSKLIPIESIFKDGSITVTNKPIVNIEIGKVKVKAGDKIKVPVEIKDIPSIGINNCNFTLKYNSNVLKYVSNEAGTIVPAPLANLSINKPDEGIIKLLFSDASQGGMPIKDNGIFVNLEFQAVNDANIGVYGLELDTIGAFSGISSAKMTSIEPQFNNGSIEIFNSAQTPVPSNTEVQTPTNTISVTPTNNSTPTNNSTPKPNPLYNLNVNIGEISGEAGGVIEVPIEFKNVPDFGINNCDFSVKYDKSIFEYVTYEAGSIVKDSIVNLACMENSGIINLLFNDATQSSSPIKNNGVFAKLKFKINSNAASGTYQINAEGYGKFSGNLNGKLTSINPIFENGIINIGNVTVKPTSTPADSSTITPTATPTATPTIKGTPTVTPIYWMNVLIGNMNAAIGEEVVVPIEFKNVPPFGINNCDFKLVYDSNALELKKVEAGDIVPEPLANLSSNKSEGKIQFLFNDASQGSMQIENGGVFAKITFKVKSTAASGIYNIRKDSVGSFSGLIDNKMTSIGPKFTDGSIVVGTVTPTATATPSAIVTTITPTATTKPIATPTIKGTPTATPMYWMNVVIGKMNAEVGGEVVVPIEFNNVPSFGINNCDFKLVYDATALELKNVEAGDIIKTPLANFSNNKSEEGKISFLFNDASQGSMQIENGGVFAKITFKVKSTTATGVYDLRKDLVGSFSGLKDNKMTSIGAEFTNGSITVAATAPTVTPTVNATPSAATPTVTPTATATPSVTIPTVTPTATATPSVTIPTVTPTATATPSAATPTVTPTATATPSVTIPTVTPTVTATPSDTIPTVTPTATATPSAIVTTITPTATAKPIATPTIKGTPTATPMYWMNVVIGKMNAEVGGEVVVPIEFKNVPSFGINNCDFKLVYDATALELKNVEAGDIIKTPLANFSNNKSEEGKISFLFNDASQGSMQIENGGVSAKITFKVKSTTAIGVYDIRKDLIGSFSGLKDSKMTSIGAEFTNGSITVATTAPTVTPTATATPSVTIPTVTPTATATPGTATPGTATPTATATPGAATPTETATPSVMIPTVTPTATATPTATATPTVKGTPTIKPVYKMNVVIGRVNVVAGEEVVVPVEFKNIPAIGVNNCNFVLEYDANVLEVKKVDAGEIVPDALINFGSNNSDEGKVYFLFNDALQGRMQIANDGIFANITFKVKSSAAAGIYNIRKDSVGAFSGLVDKLVPISAEFTDGSISVESAKSTPTATATGTNVTPTVAATVTPTATPASTTPTATPTATSTVKGTPTATPLYSMNVIIGKVNAEASGEVVVPVEFKDVPSIGINNCNFILEYDASALELDSAEAGEIVPVPLGNFSSNNKDEGKIYFLFSDGTQGRMQIVNDGIFAKIKFKVKSTASDGTYYIRKDSVGAFSGLIEKKIIKIGAEFTDGSITVRSLTPTPTVTPNVASPTPTKVVAEPTSNQPAGPGPITGTIPTATTTATATPTKASVATATPTATPIVVVEPTIVRPGYNKDADLAVFISSDKSRYEESSIITYSIEYKNIGKVNATNVKIAAQIPKFTKVYDAAKGAVKGSEIVWMIGNLAVGESYTKEYKVKVDSLTKSEEYTDNTVTISSDQTVDIPENITTGNDDKSTIRVMLYSNRFTPGSHSSYILGYKDKTFKPKQNVTRAEVAAMFARIMGLTVKDGAKSSYKDVSNKHWALKYIEAVTKSGIFKGYKDSTFHPNAPITRAELSTVIFNYLHLNNIAPSKVHFTDINKHWAKNYIEEIYRFKLIQGYSDGSFKPNNNITRAEVVTMINRMLYRGPLKVKVGSFPDVSPKYWAYGDIEEASRNHKYTRDEKDGSEILIE

SEQ ID NO: 2 - protein, 63 aa, artificial sequence
  synthetic peptide
GDVNDDGKVNSTDLTLLKRYVLKAVSTLPSSKAEKNADVNRDGRVDVTILSRYLIRVIEKLPI

SEQ ID NO: 3 - protein, 1023 aa, artificial sequence
  synthetic peptide
ATGGGCAGCAGCCATCATCATCATCATCACAGCAGCGGCCTGGTGCCGCGCGGCAGCCATATGGCTAGTGGCAATGCAACACCGACCAAGGGAGCAACACCAACAAATACAGCTACGCCGACAAAATCAGCTACGGCTACGCCCACCAGGCCATCGGTACCGACAAACACACCGACAAACACACCGGCAAATACACCGGTATCAGGCAATTTGAAGGTTGAATTCTACAACAGCAATCCTTCAGATACTACTAACTCAATCAATCCTCAGTTCAAGGTTACTAATACCGGAAGCAGTGCAATTGATTTGTCCAAACTCACATTGAGATATTATTATACAGTAGACGGACAGAAAGATCAGACCTTCTGGTGTGACCATGCTGCAATAATCGGCAGTAACGGCAGCTACAACGGAATTACTTCAAATGTAAAAGGAACATTTGTAAAAATGAGTTCCTCAACAAATAACGCAGACACCTACCTTGAAATAAGCTTTACAGGCGGAACTCTTGAACCGGGTGCACATGTTCAGATACAAGGTAGATTTGCAAAGAATGACTGGAGTAACTATACACAGTCAAATGACTACTCATTCAAGTCTGCTTCACAGTTTGTTGAATGGGATCAGGTAACAGCATACTTGAACGGTGTTCTTGTATGGGGTAAAGAACCCGGTGGCAGTGTAGTACCATCAACACAGCCTGTAACAACACCACCTGCAACAACAAAACCACCTGCAACAACAAAACCACCTGCAACAACAATACCGCCGTCAGATGATCCGAATGCAGCTAGCAATTCTCCTCAAAATGAAGTACTGTACGGAGATGTGAATGATGACGGAAAAGTAAACTCCACTGACTTGACTTTGTTAAAAAGATATGTTCTTAAAGCCGTCTCAACTCTCCCTTCTTCCAAAGCTGAAAAGAACGCAGATGTAAATCGTGACGGAAGAGTTAATTCCAGTGATGTCACAATACTTTCAAGATATTTGATAAGGGTAATCGAGAAATTACCAATATAA

SEQ ID NO: 4 - protein, 340 aa, artificial sequence
  synthetic peptide
MGSSHHHHHHSSGLVPRGSHMASGNATPTKGATPTNTATPTKSATATPTRPSVPTNTPTNTPANTPVSGNLKVEFYNSNPSDTTNSINPQFKVTNTGSSAIDLSKLTLRYYYTVDGQKDQTFWCDHAAIIGSNGSYNGITSNVKGTFVKMSSSTNNADTYLEISFTGGTLEPGAHVQIQGRFAKNDWSNYTQSNDYSFKSASQFVEWDQVTAYLNGVLVWGKEPGGSVVPSTQPVTTPPATTKPPATTKPPATTIPPSDDPNAASNSPQNEVLYGDVNDDGKVNSTDLTLLKRYVLKAVSTLPSSKAEKNADVNRDGRVNSSDVTILSRYLIRVIEKLPI

SEQ ID NO: 5 - protein, 450 aa, artificial sequence
  synthetic peptide
TTPVTTPTTTDDLDALEASQIGNMISIWVSHSIHTGNQHQSEPISNTNFLTEKAVASVKLAGNSSLCPINGWAVYSKDNSIRIGSKGDVFVIREPFISCSHLECRTFFLTQGALLNDKHSNGTVKDRSPHRTLMSCPVGEAPSPYNSRFESVAWSASACHDGTSWLTIGISGPDNGAVAVLKYNGIITDTIKSWRNNILRTQESECACVNGSCFTVMTDGPSNGQASHKIFKMEKGKVVKSVELDAPNYHYEECSCYPNAGEITCVCRDNWHGSNRPWVSFNQNLEYQIGYICSGVFGDNPRPNDGTGSCGPVSSNGAYGVKGFSFKYGNGVWIGRTKSTNSRSGFEMIWDPNGWTETDSSFSVKQDIVAITDWSGYSGSFVQHPELTGLDCIRPCFWVELIRGRPKESTIWTSGSSISFCGVNSDTVGWSWPDGAELPFTIDKHHHHHH

SEQ ID NO: 6 - protein, 255 aa, artificial sequence
  synthetic peptide
MGSSHHHHHHSSGLVPRGSHMASIERLSSGLRINSAKDDAAGQAIANRFTANIKGLTQASRNANDGISIAQTTEGALNEINNNLQRVRELAVQSANSTNSQSDLDSIQAEITQRLNEIDRVSGQTQFNGVKVLAQDNTLTIQVGANDGETIDIDLKQINSQTLGLDSLNVQASQPELAEAAAKTTENPLQKIDAALAQVDALRSDLGAVQNRFNSAITNLGNTVNNLSEARSRIEDSDYATEVSNMSRAQILQAS

SEQ ID NO: 7 - protein, 518 aa, artificial sequence
  synthetic peptide
MDLDAVRIKVDTVNAKPGDTVNIPVRFSGIPSKGIANCDFVYSYDPNVLEIIEIKPGELIVDPNPTKSFDTAVYPDRKMIVFLFAEDSGTGAYAITKDGVFATIVAKVKEGAPNGLSVIKFVEVGGFANNDLVEQKTQFFDGGVNVGDTTEPATPTTPVTTPTTTDDLDAASEGGSLAALTAHQACHLPLETFTRHRQPRGWEQLEQCGYPVQRLVALYLAARLSWNQVDQVIRNALASPGSGGDLGEAIREQPEQARLALTLAAAESERFVRQGTGNDEAGAANGPADSGDALLERNYPTGAEFLGDGGDVSFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRSSLPGFYRTSLTLAAPEAAGEVERLIGHPLPLRLDAITGPEEEGGRLETILGWPLAERTVVIPSAIPTDPRNVGGDLDPSSIPDKEQAISALPDYASQPGKPPREDLK

SEQ ID NO: 8 - protein, 657 aa, artificial sequence
  synthetic peptide
MGSSHHHHHHSSGLVPRGSHMASMDLDAVRIKVDTVNAKPGDTVNIPVRFSGIPSKGIANCDFVYSYDPNVLEIIEIKPGELIVDPNPTKSFDTAVYPDRKMIVFLFAEDSGTGAYAITKDGVFATIVAKVKEGAPNGLSVIKFVEVGGFANNDLVEQKTQFFDGGVNVGDTTEPATPTTPVTTPTTTDDLDAASELEVRRVRQAFLSGRSRPLRFRLQQLEALRRMVQEREKDILTAIAADLCKSEFNVYSQEVITVLGEIDFMLENLPEWVTAKPVKKNVLTMLDEAYIQPQPLGVVLIIGAWNYPFVLTIQPLIGAIAAGNAVIIKPSELSENTAKILAKLLPQYLDQDLYIVINGGVEETTELLKQRFDHIFYTGNTAVGKIVMEAAAKHLTPVTLELGGKSPCYIDKDCDLDIVCRRITWGKYMNCGQTCIAPDYILCEASLQNQIVWKIKETVKEFYGENIKESPDYERIINLRHFKRILSLLEGQKIAFGGETDEATRYIAPTVLTDVDPKTKVMQEEIFGPILPIVPVKNVDEAINFINEREKPLALYVFSHNHKLIKRMIDETSSGGVTGNDVIMHFTLNSFPFGGVGSSGMGAYHGKHSFDTFSHQRPCLLKSLKREGANKLRYPPNSQSKVDWGKFFLLKRFNKEK

SEQ ID NO: 9 - protein, 492 aa, artificial sequence
  synthetic peptide
MGSSHHHHHHSSGLVPRGSHMASMDLDAVRIKVDTVNAKPGDTVNIPVRFSGIPSKGIANCDFVYSYDPNVLEIIEIKPGELIVDPNPTKSFDTAVYPDRKMIVFLFAEDSGTGAYAITKDGVFATIVAKVKEGAPNGLSVIKFVEVGGFANNDLVEQKTQFFDGGVNVGDTTEPATPTTPVTTPTTTDDLDAASSLYPSLEDLKVDKVIQAQTAFSANPANPAILSEASAPIPHDGNLYPRLYPELSQYMGLSLNEEEIRANVAVVSGAPLQGQLVARPSSINYMVAPVTGNDVGIRRAEIKQGIREVILCKDQDGKIGLRLKSIDNGIFVQLVQANSPASLVGLRFGDQVLQINGENCAGWSSDKAHKVLKQAFGEKITMTIRDRPFERTITMHKDSTGHVGFIFKNGKITSIVKDSSAARNGLLTEHNICEINGQNVIGLKDSQIADILSTSGTVVTITIMPAFIFEHIIKRMAPSIMKSLMDHTIPEV

SEQ ID NO: 10 - protein, 291 aa, artificial sequence
  synthetic peptide
MGSSHHHHHHSSGLVPRGSHMASMDLDAVRIKVDTVNAKPGDTVNIPVRFSGIPSKGIANCDFVYSYDPNVLEIIEIKPGELIVDPNPTKSFDTAVYPDRKMIVFLFAEDSGTGAYAITKDGVFATIVAKVKEGAPNGLSVIKFVEVGGFANNDLVEQKTQFFDGGVNVGDTTEPATPTTPVTTPTTTDDLDAASEEISLANLDTNKLEAIAQEIYVDLIEDSCLGFCFEVHRAVKCGYFYLEFAETGSVKDFGIQPVEDKGACRLPLCSLPGEPGNGPDQQLQRSPPEFQ

SEQ ID NO: 11 - protein, 513 aa, artificial sequence
  synthetic peptide
MDLDAVRIKVDTVNAKPGDTVNIPVRFSGIPSKGIANCDFVYSYDPNVLEIIEIKPGELIVDPNPTKSFDTAVYPDRKMIVFLFAEDSGTGAYAITKDGVFATIVAKVKEGAPNGLSVIKFVEVGGFANNDLVEQKTQFFDGGVNVGDTTEPATPTTPVTTPTTTDDLDAASLEPEPEPEPEPVKEEKLSPEPILVDTASPSPMETSGCAPAEEDLCQAFSDVILAVNDVDAEDGADPNLCSEYVKDIYAYLRQLEEEQAVRPKYLLGREVTGNMRAILIDWLVQVQMKFRLLQETMYMTVSIIDRFMQNNCVPKKMLQLVGVTAMFIASKYEEMYPPEIGDFAFVTDNTYTKHQIRQMEMKILRALNFGLGRPLPLHFLRRASKIGEVDVEQHTLAKYLMELTMLDYDMVHFPPSQIAAGAFCLALKILDNGEWTPTLQHYLSYTEESLLPVMQHLAKNVVMVNQGLTKHMTVKNKYATSKHAKISTLPQLNSALVQDLAKAVAKVHHHHHH

SEQ ID NO: 12 - protein, 376 aa, artificial sequence
  synthetic peptide
MDLDAVRIKVDTVNAKPGDTVNIPVRFSGIPSKGIANCDFVYSYDPNVLEIIEIKPGELIVDPNPTKSFDTAVYPDRKMIVFLFAEDSGTGAYAITKDGVFATIVAKVKEGAPNGLSVIKFVEVGGFANNDLVEQKTQFFDGGVNVGDTTEPATPTTPVTTPTTTDDLDAASLEMALRVTRNSKINAENKAKINMAGAKRVPTAPAATSKPGLRPRTALGDIGNKVSEQLQAKMPMKKEAKPSATGKVIDKKLPKPLEKVPMLVPVPVSEPVPEPEPEPEPEPVKEEKLSPEPILVDTASPSPMETSGCAPAEEDLCQAFSDVILAVNDVDAEDGADPNLCSEYVKDIYAYLRQLEEEQAVRPKYLLGREHHHHHH

SEQ ID NO: 13 - protein, 475 aa, artificial sequence [sequence omitted]

SEQ ID NO: 14 - protein, 204 aa, artificial sequence
  synthetic peptide
MDLDAVRIKVDTVNAKPGDTVNIPVRFSGIPSKGIANCDFVYSYDPNVLEIIEIKPGELIVDPNPTKSFDTAVYPDRKMIVFLFAEDSGTGAYAITKDGVFATIVAKVKEGAPNGLSVIKFVEVGGFANNDLVEQKTQFFDGGVNVGDTTEPATPTTPVTTPTTTDDLDAARSAFTIMDQVPFSVSVSASRKGAAALEHHHHHH

SEQ ID NO: 15 - protein, 204 aa, artificial sequence
  synthetid peptide
MDLDAVRIKVDTVNAKPGDTVNIPVRFSGIPSKGIANCDFVYSYDPNVLEIIEIKPGELIVDPNPTKSFDTAVYPDRKMIVFLFAEDSGTGAYAITKDGVFATIVAKVKEGAPNGLSVIKFVEVGGFANNDLVEQKTQFFDGGVNVGDTTEPATPTTPVTTPTTTDDLDAARTAEELAGIGILTVILGASRKGAAALEHHHHHH

SEQ ID NO: 16 - protein, 398 aa, artificial sequence
  synthetic peptide
LDITSHHHHHHDDLDAVRIKVDTVNAKPGDTVRIPVRFSGIPSKGIANCDFVYSYDPNVLEIIEIEPGDIIVDPNPDKSFDTAVYPDRKIIVFLFAEDSGTGAYAITKDGVFATIVAKVKEGAPNGLSVIKFVEVGGFANNDLVEQKTQFFDGGVNVGDTTEPATPTTPVTTPTTTDDLDAASQTPTNTISVTPTNNSTPTNNSNPKPNPASMQAEGRGTGGSTGDADGPGGPGIPDGPGGNAGGPGEAGATGGRGPRGAGAARASGPGGGAPRGPHGGAASGLNGCCRCGARGPESRLLEFYLAMPFATPMEAELARRSLAQDAPPLPVPGVLLKEFTVSGNILTIRLTAADHRQLQLSISSCLQQLSLLMWITQCFLPVFLAQPPSGQRRHHHHHH

SEQ ID NO: 17 - protein, 447 aa, artificial sequence
  synthetic peptide
LDITSHHHHHHDDLDAVRIKVDTVNAKPGDTVRIPVRFSGIPSKGIANCDFVYSYDPNVLEIIEIEPGDIIVDPNPDKSFDTAVYPDRKIIVFLFAEDSGTGAYAITKDGVFATIVAKVKEGAPNGLSVIKFVEVGGFANNDLVEQKTQFFDGGVNVGDTTEPATPTTPVTTPTTTDDLDAASDTTEPATPTTPVTTPTTTLLAPLILSRIVGGWECEKHSQPWQVLVASRGRAVCGGVLVHPQWVLTAAHCIRNKSVILLGRHSLFHPEDTGQVFQVSHSFPHPLYDMSLLKNRFLRPGDDSSHDLMLLRLSEPAELTDAVKVMDLPTQEPALGTTCYASGWGSIEPEEFLTPKKLQCVDLHVISNDVCAQVHPQKVTKFMLCAGRWTGGKSTCSGDSGGPLVCNGVLQGITSWGSEPCALPERPSLYTKVVHYRKWIKDTIVANP

SEQ ID NO: 18 - protein, 321 aa, artificial sequence
  synthetic peptide
LDDLDAVRIKVDTVNAKPGDTVRIPVRFSGIPSKGIANCDFVYSYDPNVLEIIEIEPGDIIVDPNPDKSFDTAVYPDRKIIVFLFAEDSGTGAYAITKDGVFATIVAKVKEGAPNGLSVIKFVEVGGFANNDLVEQKTQFFDGGVNVGDTTEPATPTTPVTTPTTTDDLDALEMGAPTLPPAWQPFLKDHRISTFKNWPFLEGCACTPERMAEAGFIHCPTENEPDLAQCFFCFKELEGWEPDDDPIEEHKKHSSGCAFLSVKKQFEELTLGEFLKLDRERAKNKIAKETNNKKKEFEETAKKVRRAIEQLAAMDHHHHHH

SEQ ID NO: 19 - protein, 750 aa, artificial sequence
  synthetic peptide
GLDDLDAVRIKVDTVNAKPGDTVRIPVRFSGIPSKGIANCDFVYSYDPNVLEIIEIEPGDIIVDPNPDKSFDTAVYPDRKIIVFLFAEDSGTGAYAITKDGVFATIVAKVKEGAPNGLSVIKFVEVGGFANNDLVEQKTQFFDGGVNVGDTTEPATPTTPVTTPTTTDDLDALEKVPRNQDWLGVSRQLRTKAWNRQLYPEWTEAQRLDCWRGGQVSLKVSNDGPTLIGANASFSIALNFPGSQKVLPDGQVIWVNNTIINGSQVWGGQPVYPQETDDACIFPDGGPCPSGSWSQKRSFVYVWKTWGQYWQVLGGPVSGLSIGTGRAMLGTHTMEVTVYHRRGSRSYVPLAHSSSAFTITDQVPFSVSVSQLRALDGGNKHFLRNQPLTFALQLHDPSGYLAEADLSYTWDFGDSSGTLISRAPVVTHTYLEPGPVTAQVVLQAAIPLTSCGSSPVPGTTDGHRPTAEAPNTTAGQVPTTEVVGTTPGQAPTAEPSGTTSVQVPTTEVISTAPVQMPTAESTGMTPEKVPVSEVMGTTLAEMSTPEATGMTPAEVSIVVLSGTTAAQVTTTEWVETTARELPIPEPEGPDASSIMSTESITGSLGPLLDGTATLRLVKRQVPLDCVLYRYGSFSVTLDIVQGIESAEILQAVPSGEGDAFELTVSCQGGLPKEACMEISSPGCQPPAQRLCQPVLPSPACQLVLHQILKGGSGTYCLNVSLADTNSLAVVSTQLIVPGILLTGQEAGLGQ

SEQ ID NO: 20 - protein, 202 aa, artificial sequence
  synthetic peptide
MDLDAVRIKVDTVNAKPGDTVNIPVRFSGIPSKGIANCDFVYSYDPNVLEIIEIKPGELIVDPNPTKSFDTAVYPDRKMIVFLFAEDSGTGAYAITKDGVFATIVAKVKEGAPNGLSVIKFVEVGGFANNDLVEQKTQFFDGGVNVGDTTEPATPTTPVTTPTTTDDLDAASMTGGQQMGRDPNSSSVDKLAAALEHHHHHH

SEQ ID NO: 21 - protein, 220 aa, artificial sequence
  synthetic peptide
MDLDAVRIKVDTVNAKPGDTVNIPVRFSGIPSKGIANCDFVYSYDPNVLEIIEIKPGELIVDPNPTKSFDTAVYPDRKMIVFLFAEDSGTGAYAITKDGVFATIVAKVKEGAPNGLSVIKFVEVGGFANNDLVEQKTQFFDGGVNVGDTTEPATPTTPVTTPTTTDDLDAASMTGGQQMGRDPNSSLRPHSSTTTTTTEIRLLTKPERKLSWLLPPLSNN

SEQ ID NO: 22 - protein, 397 aa, artificial sequence
  synthetic peptide
MGSSHHHHHHSSGLVPRGSHMASMDLDAVRIKVDTVNAKPGDTVNIPVRFSGIPSKGIANCDFVYSYDPNVLEIIEIKPGELIVDPNPTKSFDTAVYPDRKMIVFLFAEDSGTGAYAITKDGVFATIVAKVKEGAPNGLSVIKFVEVGGFANNDLVEQKTQFFDGGVNVGDTTEPATPTTPVTTPTTTDDLDAASVLDSHYQDVLKEVKAAASKVKANALYDVVSKLPLAVMGSSYGFQYSPGQRVEFLVQAWKSKKTPMGFSYDTRCFDSTVTESDIRTEEAIYQCCDLDPQARVAIKSLTERLYVGRCRASGVLTTSCGNTLTCYIKARAACRAAGLQDCTMLVCGDDLVVICESAGVQEDAASLRAFTEAMTRYSAPPGDPPQPEYDLELITAS

SEQ ID NO: 23 - protein, 548 aa, artificial sequence
  synthetic peptide
MGSSHHHHHHSSGLVPRGSHMASMDLDAVRIKVDTVNAKPGDTVNIPVRFSGIPSKGIANCDFVYSYDPNVLEIIEIKPGELIVDPNPTKSFDTAVYPDRKMIVFLFAEDSGTGAYAITKDGVFATIVAKVKEGAPNGLSVIKFVEVGGFANNDLVEQKTQFFDGGVNVGDTTEPATPTTPVTTPTTTDDLDAASSMSYSWTGALVTPCAAEAALVYSTTSRSACQRQKKVTFDRLQVLDSHYQDVLKEVKAAASKVKANLLSVEEACSLTPPHSAKSKFGYGAKDVRCHARKAVNHINSVWKDLLEDSVTPIDTTIMAKNEVFCVQPEKGGRKPARLIVFPDLGVRVCEKMALYDVVSKLPLAVMGSSYGFQYSPGQRVEFLVQAWKSKKTPMGFSYDTRCFDSTVTESDIRTEEAIYQCCDLDPQARVAIKSLTERLYVGGPLTNSRGENCGYRRCRASGVLTTSCGNTLTCYIKARAACRAAGLQDCTMLVCGDDLVVICESAGVQEDAASLRAFTEAMTRYSAPPGDPPQPEYDLELITSCSAS

SEQ ID NO: 24 - protein, 260 aa, artificial sequence
  synthetic peptide
MGSSHHHHHHSSGLVPRGSHMASMDLDAVRIKVDTVNAKPGDTVNIPVRFSGIPSKGIANCDFVYSYDPNVLEIIEIKPGELIVDPNPTKSFDTAVYPDRKMIVFLFAEDSGTGAYAITKDGVFATIVAKVKEGAPNGLSVIKFVEVGGFANNDLVEQKTQFFDGGVNVGDTTEPATPTTPVTTPTTTDDLDAASVGQLFTFSPRRHWTTQDCNCSIYPGHITGHRMAWDMMMNWSPTTAVVAQLLRIPQAILDMIAGAS

SEQ ID NO: 25 - protein, 539 aa, artificial sequence
  synthetic peptide
MGSSHHHHHHSSGLVPRGSHMASMDLDAVRIKVDTVNAKPGDTVNIPVRFSGIPSKGIANCDFVYSYDPNVLEIIEIKPGELIVDPNPTKSFDTAVYPDRKMIVFLFAEDSGTGAYAITKDGVFATIVAKVKEGAPNGLSVIKFVEVGGFANNDLVEQKTQFFDGGVNVGDTTEPATPTTPVTTPTTTDDLDAASETHVTGGSAARTTAGLAGLFTPGAKQNIQLINTNGSWHINRTALNCNDSLNTGWVAGLFYYHKFNSSGCPERLASCRPLTDFDQGWGPISYANGSGPDQRPYCWHYPPKPCGIVPAKSVCGPVYCFTPSPVVVGTTDRSGAPTYNWGENDTDVFVLNNTRPPLGNWFGCTWMNSTGFTKVCGAPPCVIGGVGNNTLHCPTDCFRKHPEATYSRCGSGPWITPRCLVDYPYRLWHYPCTINYTIFKIRMYVGGVEHRLEAACNWTRGERCDLEDRDRSELSPLLLSTTQWQVLPCSFTTLPALSTGLIHLHQNIVDVQYLYGVGSSIASWAIKWEYVVLLFLLAS

SEQ ID NO: 26 - protein, 400 aa, artificial sequence
  synthetic peptide
MGSSHHHHHHSSGLVPRGSHMASMDLDAVRIKVDTVNAKPGDTVNIPVRFSGIPSKGIANCDFVYSYDPNVLEIIEIKPGELIVDPNPTKSFDTAVYPDRKMIVFLFAEDSGTGAYAITKDGVFATIVAKVKEGAPNGLSVIKFVEVGGFANNDLVEQKTQFFDGGVNVGDTTEPATPTTPVTTPTTTDDLDAASAPITAYAQQTRGLLGCIITSLTGRDKNQVEGEVQIVSTAAQTFLATCINGVCWTVYHGAGTRTIASPKGPVIQMYTNVDQDLVGWPAPQGARSLTPCTCGSSDLYLVTRHADVIPVRRRGDSRGSLLSPRPISYLKGSSGGPLLCPAGHAVGIFRAAVCTRGVAKAVDFIPVENLETTMRSPVFTDNSSPPAVPQSASSTPEFAA

SEQ ID NO: 27 - protein, 641 aa, artificial sequence
  synthetic peptide
MGSSHHHHHHSSGLVPRGSHMASMDLDAVRIKVDTVNAKPGDTVNIPVRFSGIPSKGIANCDFVYSYDPNVLEIIEIKPGELIVDPNPTKSFDTAVYPDRKMIVFLFAEDSGTGAYAITKDGVFATIVAKVKEGAPNGLSVIKFVEVGGFANNDLVEQKTQFFDGGVNVGDTTEPATPTTPVTTPTTTDDLDAASFQVAHLHAPTGSGKSTKVPAAYAAQGYKVLVLNPSVAATLGFGAYMSKAHGIDPNIRTGVRTITTGSPITYSTYGKFLADGGCSGGAYDIIICDECHSTDATSILGIGTVLDQAETAGARLVVLATATPPGSVTVPHPNIEEVALSTTGEIPFYGKAIPLEVIKGGRHLIFCHSKKKCDELAAKLVALGINAVAYYRGLDVSVIPTSGVVVVVATDALMTGFTGDFDSVIDCNTCVTQTVDFSLDPTFTIETTTLPQDAVSRTQRRGRTGRGKPGIYRFVAPGERPSGMFDSSVLCECYDAGCAWYELTPAETTVRLRAYMNTPGLPVCQDHLEFWEGVFTGLTHIDAHFLSQTKQSGENLPYLVAYQATVCARAQAPPPSWDQMWKCLIRLKPTLHGPTPLLYRLGAVQNEVTLTHPITKYIMTCMSADLEVVTAAALEHHHHHH

SEQ ID NO: 28 - protein, 216 aa, artificial sequence
  synthetic peptide
MGSSHHHHHHSSGLVPRGSHMASMDLDAVRIKVDTVNAKPGDTVNIPVRFSGIPSKGIANCDFVYSYDPNVLEIIEIKPGELIVDPNPTKSFDTAVYPDRKMIVFLFAEDSGTGAYAITKDGVFATIVAKVKEGAPNGLSVIKFVEVGGFANNDLVEQKTQFFDGGVNVGDTTEPATPTTPVTTPTTTDDLDAASEKIRLRPGGKKKYKLKHIVAS

SEQ ID NO: 29 - protein, 229 aa, artificial sequence
  synthetic peptide
MGSSHHHHHHSSGLVPRGSHMASMDLDAVRIKVDTVNAKPGDTVNIPVRFSGIPSKGIANCDFVYSYDPNVLEIIEIKPGELIVDPNPTKSFDTAVYPDRKMIVFLFAEDSGTGAYAITKDGVFATIVAKVKEGAPNGLSVIKFVEVGGFANNDLVEQKTQFFDGGVNVGDTTEPATPTTPVTTPTTTDDLDAASNPPIPVGEIYKRWIILGLNKIVRMYSPTSILDAS

SEQ ID NO: 30 - protein, 227 aa, artificial sequence
  synthetic peptide
MGSSHHHHHHSSGLVPRGSHMASMDLDAVRIKVDTVNAKPGDTVNIPVRFSGIPSKGIANCDFVYSYDPNVLEIIEIKPGELIVDPNPTKSFDTAVYPDRKMIVFLFAEDSGTGAYAITKDGVFATIVAKVKEGAPNGLSVIKFVEVGGFANNDLVEQKTQFFDGGVNVGDTTEPATPTTPVTTPTTTDDLDAASHTQGYFPDWQNYTPGPGVRYPLTFGWLYKLAS

SEQ ID NO: 31 - protein, 229 aa, artificial sequence
  synthetic peptide
MGSSHHHHHHSSGLVPRGSHMASMDLDAVRIKVDTVNAKPGDTVNIPVRFSGIPSKGIANCDFVYSYDPNVLEIIEIKPGELIVDPNPTKSFDTAVYPDRKMIVFLFAEDSGTGAYAITKDGVFATIVAKVKEGAPNGLSVIKFVEVGGFANNDLVEQKTQFFDGGVNVGDTTEPATPTTPVTTPTTTDDLDAASVGFPVTPQVPLRPMTYKAAVDLSHFLKEKGGLAS

SEQ ID NO: 32 - protein, 228 aa, artificial sequence
  synthetic peptide
MGSSHHHHHHSSGLVPRGSHMASMDLDAVRIKVDTVNAKPGDTVNIPVRFSGIPSKGIANCDFVYSYDPNVLEIIEIKPGELIVDPNPTKSFDTAVYPDRKMIVFLFAEDSGTGAYAITKDGVFATIVAKVKEGAPNGLSVIKFVEVGGFANNDLVEQKTQFFDGGVNVGDTTEPATPTTPVTTPTTTDDLDAASAIFQSSMTKILEPFRKQNPDIVIYQYMDDLYAS

SEQ ID NO: 33 - protein, 316 aa, artificial sequence
  synthetic peptide
MGSSHHHHHHSSGLVPRGSHMASMDLDAVRIKVDTVNAKPGDTVNIPVRFSGIPSKGIANCDFVYSYDPNVLEIIEIKPGELIVDPNPTKSFDTAVYPDRKMIVFLFAEDSGTGAYAITKDGVFATIVAKVKEGAPNGLSVIKFVEVGGFANNDLVEQKTQFFDGGVNVGDTTEPATPTTPVTTPTTTDDLDAASLEMGARASILSGGELDRWEKIRLRPGGKKKYKLKHIVWASRELERFAVNPGLLETSEGCRQILGQLQPSLQTGSEELRSLYNTVATLYCVHQRIEIKDTKEALDKIEEEQNKSVDHHHHHH

SEQ ID NO: 34 - protein, 391 aa, artificial sequence
  synthetic peptide
LDDLDAVRIKVDTVNAKPGDTVRIPVRFSGIPSKGIANCDFVYSYDPNVLEIIEIEPGDIIVDPNPDKSFDTAVYPDRKIIVFLFAEDSGTGAYAITKDGVFATIVAKVKEGAPNGLSVIKFVEVGGFANNDLVEQKTQFFDGGVNVGDTTEPATPTTPVTTPTTTDDLDALEVHQAISPRTLNAWVKVVEEKAFSPEVIPMFSALSEGATPQDLNTMLNTVGGHQAAMQMLKETINEEAAEWDRVHPVHAGPIAPGQMREPRGSDIAGTTSTLQEQIGWMTNNPPIPVGEIYKRWIILGLNKIVRMYSPTSILDIRQGPKEPFRDYVDRFYKTLRAEQASQEVKNWMTETLLVQNANPDCKTILKALGPAATLEEMMTACQGVGHHHHHH

SEQ ID NO: 35 - protein, 385 aa, artificial sequence
  synthetic peptide
LDDLDAVRIKVDTVNAKPGDTVRIPVRFSGIPSKGIANCDFVYSYDPNVLEIIEIEPGDIIVDPNPDKSFDTAVYPDRKIIVFLFAEDSGTGAYAITKDGVFATIVAKVKEGAPNGLSVIKFVEVGGFANNDLVEQKTQFFDGGANVGDTTEPATPTTPVTTPTTTDDLDAASMGGKWSKRSVVGWPTVRERMRRAEPAADGVGAVSRDLEKHGAITSSNTAANNADCAWLEAQEEEEVGFPVRPQVPLRPMTYKGALDLSHFLKEKGGLEGLIYSQKRQDILDLWVYHTQGYFPDWQNYTPGPGIRYPLTFGWCFKLVPVEPEKVEEANEGENNSLLHPMSLHGMDDPEREVLVWKFDSRLAFHHMARELHPEYYKDCHHHHHH

SEQ ID NO: 36 - protein, 317 aa, artificial sequence
  synthetic peptide
MGSSHHHHHHSSGLVPRGSHMASMDLDAVRIKVDTVNAKPGDTVNIPVRFSGIPSKGIANCDFVYSYDPNVLEIIEIKPGELIVDPNPTKSFDTAVYPDRKMIVFLFAEDSGTGAYAITKDGVFATIVAKVKEGAPNGLSVIKFVEVGGFANNDLVEQKTQFFDGGVNVGDTTEPATPTTPVTTPTTTDDLDAASMHQKRTAMFQDPQERPRKLPQLCTELQTTIHDIILECVYCKQQLLRREVGDFAFRDLCIVYRDGNPYAVCDKCLKFYSKISEYRHYCYSVYGTTLEQQYNKPLCDLLIRCINCQKPLCPEAS

SEQ ID NO: 37 - protein, 256 aa, artificial sequence
  synthetic peptide
MGSSHHHHHHSSGLVPRGSHMASMDLDAVRIKVDTVNAKPGDTVNIPVRFSGIPSKGIANCDFVYSYDPNVLEIIEIKPGELIVDPNPTKSFDTAVYPDRKMIVFLFAEDSGTGAYAITKDGVFATIVAKVKEGAPNGLSVIKFVEVGGFANNDLVEQKTQFFDGGVNVGDTTEPATPTTPVTTPTTTDDLDAASMHGDTPTLHEYMLDLQPETTDLYGYGQLNDSSEEEDEIDGPAGQAEPDRAHYNIVTFCCKA

SEQ ID NO: 38 - protein, 504 aa, artificial sequence
  synthetic peptide
MGSSHHHHHHSSGLVPRGSHMASMDLDAVRIKVDTVNAKPGDTVNIPVRFSGIPSKGIANCDFVYSYDPNVLEIIEIKPGELIVDPNPTKSFDTAVYPDRKMIVFLFAEDSGTGAYAITKDGVFATIVAKVKEGAPNGLSVIKFVEVGGFANNDLVEQKTQFFDGGVNVGDTTEPATPTTPVTTPTTTDDLDAASSGTGGRTGYIPLGTRPPTATDTLAPVRPPLTVDPVGPSDPSIVSLVEETSFIDAGAPTSVPSIPPDVSGFSITTSTDTTPAILDINNTVTTVTTHNNPTFTDPSVLQPPTPAETGGHFTLSSSTISTHNYEEIPMDTFIVSTNPNTVTSSTPIPGSRPVARLGLYSRTTQQVKVVDPAFVTTPTKLITYDNPAYEGIDVDNTLYFSSNDNSINIAPDPDFLDIVALHRPALTSRRTGIRYSRIGNKQTLRTRSGKSIGAKVHYYYDLSTIDPAEEIELQTITPSTYTTTSHAASPTSINNGLYDIYAAS

SEQ ID NO: 39 - protein, 264 aa, artificial sequence
  synthetic peptide
MGSSHHHHHHSSGLVPRGSHMASMDLDAVRIKVDTVNAKPGDTVNIPVRFSGIPSKGIANCDFVYSYDPNVLEIIEIKPGELIVDPNPTKSFDTAVYPDRKMIVFLFAEDSGTGAYAITKDGVFATIVAKVKEGAPNGLSVIKFVEVGGFANNDLVEQKTQFFDGGVNVGDTTEPATPTTPVTTPTTTDDLDAASMHGPKATLQDIVLHLEPQNEIPVDLLGHGQLSDSEEENDEIDGVNHQHLPARRAEPQRHTMLCMCCKAS

SEQ ID NO: 40 - protein, 378 aa, artificial sequence
  synthetic peptide
GSSHHHHHHSSGLVPRGSHMASMDLDAVRIKVDTVNAKPGDTVNIPVRFSGIPSKGIANCDFVYSYDPNVLEIIEIKPGELIVDPNPTKSFDTAVYPDRKMIVFLFAEDSGTGAYAITKDGVFATIVAKVKEGAPNGLSVIKFVEVGGFANNDLVEQKTQFFDGGVNVGDTTEPATPTTPVTTPTTTDDLDAASMHQKRTAMFQDPQERPRKLPQLCTELQTTIHDIILECVYCKQQLLRREVGDFAFRDLCIVYRDGNPYAVCDKCLKFYSKISEYRHYCYSVYGTTLEQQYNKPLCDLLIRCINCQKPLCPEASMHGDTPTLHEYMLDLQPETTDLYGYGQLNDSSEEEDEIDGPAGQAEPDRAHYNIVTFCCKAS

SEQ ID NO: 41 - protein, 379 aa, artificial sequence
  synthetic peptide
GSSHHHHHHSSGLVPRGSHMASMDLDAVRIKVDTVNAKPGDTVNIPVRFSGIPSKGIANCDFVYSYDPNVLEIIEIKPGELIVDPNPTKSFDTAVYPDRKMIVFLFAEDSGTGAYAITKDGVFATIVAKVKEGAPNGLSVIKFVEVGGFANNDLVEQKTQFFDGGVNVGDTTEPATPTTPVTTPTTTDDLDAASMARFEDPTRRPYKLPDLCTELNTSLQDIEITCVYCKTVLELTEVGEFAFKDLFVVYRDSIPHAACHKCIDFYSRIRELRHYSDSVYGDTLEKLTNTGLYNLLIRCLRCQKPLNPASMHGPKATLQDIVLHLEPQNEIPVDLLGHGQLSDSEEENDEIDGVNHQHLPARRAEPQRHTMLCMCCKAS

SEQ ID NO: 42 - protein, 310 aa, artificial sequence
  synthetic peptide
GSSHHHHHHSSGLVPRGSHMASMDLDAVRIKVDTVNAKPGDTVNIPVRFSGIPSKGIANCDFVYSYDPNVLEIIEIKPGELIVDPNPTKSFDTAVYPDRKMIVFLFAEDSGTGAYAITKDGVFATIVAKVKEGAPNGLSVIKFVEVGGFANNDLVEQKTQFFDGGVNVGDTTEPATPTTPVTTPTTTDDLDAASMARFEDPTRRPYKLPDLCTELNTSLQDIEITCVYCKTVLELTEVGEFAFKDLFVVYRDSIPHAACHKCIDFYSRIRELRHYSDSVYGDTLEKLTNTGLYNLLIRCLRCQKPLNPAS

SEQ ID NO: 43 - protein, 523 aa, artificial sequence
  synthetic peptide
MGSSHHHHHHSSGLVPRGSHMASMDLDAVRIKVDTVNAKPGDTVNIPVRFSGIPSKGIANCDFVYSYDPNVLEIIEIKPGELIVDPNPTKSFDTAVYPDRKMIVFLFAEDSGTGAYAITKDGVFATIVAKVKEGAPNGLSVIKFVEVGGFANNDLVEQKTQFFDGGVNVGDTTEPATPTTPVTTPTTTDDLDAASDTTEPATPTTPVTTDTICIGYHANNSTDTVDTVLEKNVTVTHSVNLLEDKHNGKLCKLRGVAPLHLGKCNIAGWILGNPECESLSTASSWSYIVETSSSDNGTCYPGDFIDYEELREQLSSVSSFERFEIFPKTSSWPNHDSNKGVTAACPHAGAKSFYKNLIWLVKKGNSYPKLSKSYINDKGKEVLVLWGIHHPSTSDDQQSLYQNADAYVFVGSSRYSKKFKPEIAIRPKVRDQEGRMNYYWTLVEPGDKITFEATGNLVVPRYAFAMERNAGSGIIISDTPVHDCNTTCQTPKGAINTSLPFQNIQPITIGKCPKYVKSTKLRL

SEQ ID NO: 44 - protein, 516 aa, artificial sequence
  synthetic peptide
MGSSHHHHHHSSGLVPRGSHMASMDLDAVRIKVDTVNAKPGDTVNIPVRFSGIPSKGIANCDFVYSYDPNVLEIIEIKPGELIVDPNPTKSFDTAVYPDRKMIVFLFAEDSGTGAYAITKDGVFATIVAKVKEGAPNGLSVIKFVEVGGFANNDLVEQKTQFFDGGVNVGDTTEPATPTTPVTTPTTTDDLDAASDTTEPATPTTPVTTATLCLGHHAVPNGTLVKTITDDQIEVTNATELVQSSSTGKICNNPHRILDGIDCTLIDALLGDPHCDVFQDETWDLFVERSKAFSNCYPYDVPDYASLRSLVALSGTLEFITEGFTWTGVTQNGGSNACKRGPGSGFFSRLNWLTKSGSTYPVLNVTMPNNDNFDKLYIWGVHHPSTNQEQTSLYVQASGRVTVSTRRSQQTIIPNIGSRPWVRGLSSRISIYWTIVKPGDVLVINSNGNLIAPRGYFKMRTGKSSIMRSDAPTDTCISECITPNGSIPNDKPFQNVNKITYGACPKYVKQNTLKLA

SEQ ID NO: 45 - protein, 626 aa, artificial sequence
  synthetic peptide
MGSSHHHHHHSSGLVPRGSHMASMDLDAVRIKVDTVNAKPGDTVNIPVRFSGIPSKGIANCDFVYSYDPNVLEIIEIKPGELIVDPNPTKSFDTAVYPDRKMIVFLFAEDSGTGAYAITKDGVFATIVAKVKEGAPNGLSVIKFVEVGGFANNDLVEQKTQFFDGGVNVGDTTEPATPTTPVTTPTTTDDLDAASQIGNMISIWVSHSIHTGNQHQSEPISNTNFLTEKAVASVKLAGNSSLCPINGWAVYSKDNSIRIGSKGDVFVIREPFISCSHLECRTFFLTQGALLNDKHSNGTVKDRSPHRTLMSCPVGEAPSPYNSRFESVAWSASACHDGTSWLTIGISGPDNGAVAVLKYNGIITDTIKSWRNNILRTQESECACVNGSCFTVMTDGPSNGQASHKIFKMEKGKVVKSVELDAPNYHYEECSCYPNAGEITCVCRDNWHGSNRPWVSFNQNLEYQIGYICSGVFGDNPRPNDGTGSCGPVSSNGAYGVKGFSFKYGNGVWIGRTKSTNSRSGFEMIWDPNGWTETDSSFSVKQDIVAITDWSGYSGSFVQHPELTGLDCIRPCFWVELIRGRPKESTIWTSGSSISFCGVNSDTVGWSWPDGAELPFTIDKHHHHHH

SEQ ID NO: 46 - protein, 430 aa, artificial sequence
  synthetic peptide
MDLDAVRIKVDTVNAKPGDTVNIPVRFSGIPSKGIANCDFVYSYDPNVLEIIEIKPGELIVDPNPTKSFDTAVYPDRKMIVFLFAEDSGTGAYAITKDGVFATIVAKVKEGAPNGLSVIKFVEVGGFANNDLVEQKTQFFDGGVNVGDTTEPATPTTPVTTPTTTDDLDAASLLTEVETYVLSIIPSGPLKAEIAQRLEDVFAGKNTDLEVLMEWLKTRPILSPLTKGILGFVFTLTVPSERGLQRRRFVQNALNGNGDPNNMDKAVKLYRKLKREITFHGAKEIALSYSAGALASCMGLIYNRMGAVTTEVAFGLVCATCEQIADSQHRSHRQMVTTTNPLIRHENRMVLASTTAKAMEQMAGSSEQAAEAMDIASQARQMVQAMRTIGTHPSSSAGLKDDLLENLQAYQKRMGVQMQRFKLEHHHHHH

SEQ ID NO: 47 - protein, 512 aa, artificial sequence
  synthetic peptide
HHHHHHSSGLVPRGSHMASMDLDAVRIKVDTVNAKPGDTVNIPVRFSGIPSKGIANCDFVYSYDPNVLEIIEIKPGELIVDPNPTKSFDTAVYPDRKMIVFLFAEDSGTGAYAITKDGVFATIVAKVKEGAPNGLSVIKFVEVGGFANNDLVEQKTQFFDGGVNVGDTTEPATPTTPVTTPTTTDDLDAASDTLCIGYHANNSTDTVDTVLEKNVTVTHSVNLLEDKHNGKLCKLRGVAPLHLGKCNIAGWILGNPECESLSTASSWSYIVETPSSDNGTCYPGDFIDYEELREQLSSVSSFERFEIFPKTSSWPNHDSNKGVTAACPHAGAKSFYKNLIWLVKKGNSYPKLSKSYINDKGKEVLVLWGIHHPSTSADQQSLYQNADTYVFVGSSRYSKKFKPEIAIRPKVRDQEGRMNYYWTLVEPGDKITFEATGNLVVPRYAFAMERNAGSGIIISDTPVHDCNTTCQTPKGAINTSLPFQNIHPITIGKCPKYVKSTKLRLAHHHHHH

SEQ ID NO: 48 - protein, 676 aa, artificial sequence
  synthetic peptide
MDLDAVRIKVDTVNAKPGDTVNIPVRFSGIPSKGIANCDFVYSYDPNVLEIIEIKPGELIVDPNPTKSFDTAVYPDRKMIVFLFAEDSGTGAYAITKDGVFATIVAKVKEGAPNGLSVIKFVEVGGFANNDLVEQKTQFFDGGVNVGDTTEPATPTTPVTTPTTTDDLDAASMASQGTKRSYEQMETGGERQNATEIRASVGRMVSGIGRFYIQMCTELKLSDYEGRLIQNSITIERMVLSAFDERRNRYLEEHPSAGKDPKKTGGPIYRRRDGKWVRELILYDKEEIRRIWRQANNGEDATAGLTHLMIWHSNLNDATYQRTRALVRTGMDPRMCSLMQGSTLPRRSGAAGAAVKGVGTMVMELIRMIKRGINDRNFWRGENGRRTRIAYERMCNILKGKFQTAAQRAMMDQVRESRNPGNAEIEDLIFLARSALILRGSVAHKSCLPACVYGLAVASGYDFEREGYSLVGIDPFRLLQNSQVFSLIRPNENPAHKSQLVWMACHSAAFEDLRVSSFIRGTRVVPRGQLSTRGVQIASNENMEAMDSNTLELRSRYWAIRTRSGGNTNQQRASAGQISVQPTFSVQRNLPFERATIMAAFTGNTEGRTSDMRTEIIRMMESARPEDVSFQGRGVFELSDEKATNPIVPSFDMNNEGSYFFGDNAEEYDNHHHHHH

SEQ ID NO: 49 - protein, 512 aa, artificial sequence
  synthetic peptide
LDITSHHHHHHDDLDAVRIKVDTVNAKPGDTVRIPVRFSGIPSKGIANCDFVYSYDPNVLEIIEIEPGDIIVDPNPDKSFDTAVYPDRKIIVFLFAEDSGTGAYAITKDGVFATIVAKVKEGAPNGLSVIKFVEVGGFANNDLVEQKTQFFDGGVNVGDTTEPATPTTPVTTPTTTDDLDAASDTTEPATPTTPVTTDTICIGYHANNSTDTVDTVLEKNVTVTHSVNLLEDSHNGKLCRLKGIAPLQLGKCSIAGWILGNPECESLVSKKSWSYIAETPNSENGTCYPGYFADYEELREQLSSVSSFERFEIFPKERSWPKHNVTRGVTASCSHKGKSSFYRNLLWLTEKNGSYPNLSKSYVNNKEKEVLVLWGVHHPSNIEDQKTIYRKENAYVSVVSSNYNRRFAPEIAERPKVRGQAGRINYYWTLLEPGDTIIFEANGNLIAPWHAFALNRGFGSGIITSNASMDECDTKCQTPQGAINSSLPFQNIHPVTIGECPKYVRSTKLRMV

SEQ ID NO: 50 - protein, 504 aa, artificial sequence
  synthetic peptide
LDITSHHHHHHDDLDAVRIKVDTVNAKPGDTVRIPVRFSGIPSKGIANCDFVYSYDPNVLEIIEIEPGDIIVDPNPDKSFDTAVYPDRKIIVFLFAEDSGTGAYAITKDGVFATIVAKVKEGAPNGLSVIKFVEVGGFANNDLVEQKTQFFDGGVNVGDTTEPATPTTPVTTPTTTDDLDAASDTTEPATPTTPVTTATLCLGHHAVPNGTLVKTITDDQIEVTNATELVQSSSTGKICNNPHRILDGIDCTLIDALLGDPHCDVFQNETWDLFVERSKAFSNCYPYDVPDYASLRSLVASSGTLEFITEGFTWTGVTQNGGSNACKRGPGSGFFSRLNWLTKSGSTYPVLNVTMPNNDNFDKLYIWGVHHPSTNQEQTSLYVQASGRVTVSTRRSQQTIIPNIGSRPWVRGLSSRISIYWTIVKPGDVLVINSNGNLIAPRGYFKMRTGKSSIMRSDAPIDTCISECITPNGSIPNDKPFQNVNKITYGACPKYVKQNTLKLA

SEQ ID NO: 51 - protein, 689 aa, artificial sequence
  synthetic peptide
LDITSHHHHHHDDLDAVRIKVDTVNAKPGDTVRIPVRFSGIPSKGIANCDFVYSYDPNVLEIIEIEPGDIIVDPNPDKSFDTAVYPDRKIIVFLFAEDSGTGAYAITKDGVFATIVAKVKEGAPNGLSVIKFVEVGGFANNDLVEQKTQFFDGGVNVGDTTEPATPTTPVTTPTTTDDLDAASMASQGTKRSYEQMETGGERQDATEIRASVGRMIGGIGRFYIQMCTELKLSDYDGRLIQNSITIERMVLSAFDERRNKYLEEHPSAGKDPKKTGGPIYRRVDGKWMRELILYDKEEIRRVWRQANNGEDATAGLTHIMIWHSNLNDATYQRTRALVRTGMDPRMCSLMQGSTLPRRSGAAGAAVKGVGTIAMELIRMIKRGINDRNFWRGENGRRTRVAYERMCNILKGKFQTAAQRAMMDQVRESRNPGNAEIEDLIFLARSALILRGSVAHKSCLPACVYGLAVASGHDFEREGYSLVGIDPFKLLQNSQVVSLMRPNENPAHKSQLVWMACHSAAFEDLRVSSFIRGKKVIPRGKLSTRGVQIASNENVETMDSNTLELRSRYWAIRTRSGGNTNQQKASAGQISVQPTFSVQRNLPFERATVMAAFSGNNEGRTSDMRTEVIRMMESAKPEDLSFQGRGVFELSDEKATNPIVPSFDMSNEGSYFFGDNAEEYDSASHHHHHH

SEQ ID NO: 52 - protein, 262 aa, artificial sequence
  synthetic peptide
LDITSHHHHHHDDLDAVRIKVDTVNAKPGDTVRIPVRFSGIPSKGIANCDFVYSYDPNVLEIIEIEPGDIIVDPNPDKSFDTAVYPDRKIIVFLFAEDSGTGAYAITKDGVFATIVAKVKEGAPNGLSVIKFVEVGGFANNDLVEQKTQFFDGGVNVGDTTEPATPTTPVTTPTTTDDLDAASQTPTNTISVTPTNNSTPTNNSNPKPNPASLLTEVETPTRSEWECRCSDSSDPASLLTEVETPIRNEWGCRCNGSSDPAS

SEQ ID NO: 53 - protein, 687 aa, artificial sequence
  synthetic peptide
DTLCIGYHANNSTDTVDTVLEKNVTVTHSVNLLEDKHNGKLCKLRGVAPLHLGKCNIAGWILGNPECESLSTASSWSYIVETPSSDNGTCYPGDFIDYEELREQLSSVSSFERFEIFPKTSSWPNHDSNKGVTAACPHAGAKSFYKNLIWLVKKGNSYPKLSKSYINDKGKEVLVLWGIHHPSTSADQQSLYQNADTYVFVGSSRYSKKFKPEIAIRPKVRDQEGRMNYYWTLVEPGDKITFEATGNLVVPRYAFAMERNAGSGIIISDTPVHDCNTTCQTPKGAINTSLPFQNIHPITIGKCPKYVKSTKLRLATGLRNIPSIQSRGLFGAIAGFIEGGWTGMVDGWYGYHHQNEQGSGYAADLKSTQNAIDEITNKVNSVIEKMNTQFTAVGKEFNHLEKRIENLNKKVDDGFLDIWTYNAELLVLLENERTLDYHDSNVKNLYEKVRSQLKNNAKEIGNGCFEFYHKCDNTCMESVKNGTYDYPKYSEEAKLNREEIDGVKLESTRIYQIASTTEPATPTTPVTTPTTTDDLDAVRIKVDTVNAKPGDTVNIPVRFSGIPSKGIANCDFVYSYDPNVLEIIEIKPGELIVDPNPTKSFDTAVYPDRKMIVFLFAEDSGTGAYAITKDGVFATIVAKVKEGAPNGLSVIKFVEVGGFANNDLVEQKTQFFDGGVNVGDTHHHHHH

SEQ ID NO: 54 - protein, 518 aa, artificial sequence
  synthetic peptide
DITSHHHHHHDDLDAVRIKVDTVNAKPGDTVRIPVRFSGIPSKGIANCDFVYSYDPNVLEIIEIEPGDIIVDPNPDKSFDTAVYPDRKIIVFLFAEDSGTGAYAITKDGVFATIVAKVKEGAPNGLSVIKFVEVGGFANNDLVEQKTQFFDGGVNVGDTTEPATPTTPVTTPTTTDDLDAASDRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHIRLSTHNVINAENAPGGPYKIGTSGSCPNITNGNGFFATMAWAVPKNDKNKTATNPLTIEVPYICTEGEDQITVWGFHSDDETQMAKLYGDSKPQKFTSSANGVTTHYVSQIGGFPNQTEDGGLPQSGRIVVDYMVQKSGKTGTITYQRGILLPQKVWCASGRSKVIKGSLPLIGEADCLHEKYGGLNKSKPYYTGEHAKAIGNCPIWVKTPLKLANGTK

SEQ ID NO: 55 - protein, 209 aa, artificial sequence
  synthetic peptide
GLDITSHHHHHHDDLDAVRIKVDTVNAKPGDTVRIPVRFSGIPSKGIANCDFVYSYDPNVLEIIEIEPGDIIVDPNPDKSFDTAVYPDRKIIVFLFAEDSGTGAYAITKDGVFATIVAKVKEGAPNGLSVIKFVEVGGFANNDLVEQKTQFFDGGVNVGDTTEPATPTTPVTTPTTTDDLDAASLLTEVETPTRSEWECRCSDSSDPAS

SEQ ID NO: 56 - protein, 209 aa, artificial sequence
  synthetic peptide
GLDITSHHHHHHDDLDAVRIKVDTVNAKPGDTVRIPVRFSGIPSKGIANCDFVYSYDPNVLEIIEIEPGDIIVDPNPDKSFDTAVYPDRKIIVFLFAEDSGTGAYAITKDGVFATIVAKVKEGAPNGLSVIKFVEVGGFANNDLVEQKTQFFDGGVNVGDTTEPATPTTPVTTPTTTDDLDAASLLTEVETPIRNEWGCRCNGSSDPAS

SEQ ID NO: 57 - protein, 517 aa, artificial sequence
  synthetic peptide
MDPKGSLSWRILLFLSLAFELSYGLDDLDAVRIKVDTVNAKPGDTVRIPVRFSGIPSKGIANCDFVYSYDPNVLEIIEIEPGDIIVDPNPDKSFDTAVYPDRKIIVFLFAEDSGTGAYAITKDGVFATIVAKVKEGAPNGLSVIKFVEVGGFANNDLVEQKTQFFDGGVNVGDTTEPATPTTPVTTPTTTDDLDALDDTICIGYHANNSTDTVDTVLEKNVTVTHSVNLLEDSHNGKLCRLKGIAPLQLGKCNIAGWLLGNPECDPLLPVRSWSYIVETPNSENGICYPGDFIDYEELREQLSSVSSFERFEIFPKESSWPNHNTNGVTAACSHEGKSSFYRNLLWLTEKEGSYPKLKNSYVNKKGKEVLVLWGIHHPPNSKEQQNLYQNENAYVSVVTSNYNRRFTPEIAERPKVRDQAGRMNYYWTLLKPGDTIIFEANGNLIAPMYAFALSRGFGSGIITSNASMHECNTKCQTPLGAINSSLPYQNIHPVTIGECPKYVRSAKLRMVHHHHHH

SEQ ID NO: 58 - protein, 682 aa, artificial sequence
  synthetic peptide
LDDLDAVRIKVDTVNAKPGDTVRIPVRFSGIPSKGIANCDFVYSYDPNVLEIIEIEPGDIIVDPNPDKSFDTAVYPDRKIIVFLFAEDSGTGAYAITKDGVFATIVAKVKEGAPNGLSVIKFVEVGGFANNDLVEQKTQFFDGGVNVGDTTEPATPTTPVTTPTTTDDLDALEDQICIGYHANNSTEQVDTIMEKNVTVTHAQDILEKKHNGKLCDLDGVKPLILRDCSVAGWLLGNPMCDEFINVPEWSYIVEKANPVNDLCYPGDFNDYEELKHLLSRINHFEKIQIIPKSSWSSHEASLGVSSACPYQGKSSFFRNVVWLIKKNSTYPTIKRSYNNTNQEDLLVLWGIHHPNDAAEQTKLYQNPTTYISVGTSTLNQRLVPRIATRSKVNGQSGRMEFFWTILKPNDAINFESNGNFIAPEYAYKIVKKGDSTIMKSELEYGNCNTKCQTPMGAINSSMPFHNIHPLTIGECPKYVKSNRLVLATGLRNSPQRERRRKKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEIHHHHHH

SEQ ID NO: 59 - protein, 517 aa, artificial sequence
  synthetic peptide
MDPKGSLSWRILLFLSLAFELSYGLDDLDAVRIKVDTVNAKPGDTVRIPVRFSGIPSKGIANCDFVYSYDPNVLEIIEIEPGDIIVDPNPDKSFDTAVYPDRKIIVFLFAEDSGTGAYAITKDGVFATIVAKVKEGAPNGLSVIKFVEVGGFANNDLVEQKTQFFDGGVNVGDTTEPATPTTPVTTPTTTDDLDALEDQICIGYHANNSTEQVDTIMEKNVTVTHAQDILEKKHNGKLCDLDGVKPLILRDCSVAGWLLGNPMCDEFINVPEWSYIVEKANPVNDLCYPGDFNDYEELKHLLSRINHFEKIQIIPKSSWSSHEASLGVSSACPYQGKSSFFRNVVWLIKKNSTYPTIKRSYNNTNQEDLLVLWGIHHPNDAAEQTKLYQNPTTYISVGTSTLNQRLVPRIATRSKVNGQSGRMEFFWTILKPNDAINFESNGNFIAPEYAYKIVKKGDSTIMKSELEYGNCNTKCQTPMGAINSSMPFHNIHPLTIGECPKYVKSNRLVLAHHHHHH

SEQ ID NO: 60 - protein, 285 aa, artificial sequence
  synthetic peptide
LDITSHHHHHHDDLDAVRIKVDTVNAKPGDTVRIPVRFSGIPSKGIANCDFVYSYDPNVLEIIEIEPGDIIVDPNPDKSFDTAVYPDRKIIVFLFAEDSGTGAYAITKDGVFATIVAKVKEGAPNGLSVIKFVEVGGFANNDLVEQKTQFFDGGVNVGDTTEPATPTTPVTTPTTTDDLDAASMRVRNSVLSGKKADELEKIRLRPNGKKKYMLKHVVWAANELDRFGLAESLLENKEGCQKILSVLAPLVPTGSENLKSLYNTVCVIWCIHAEEKVKHTEEAAS

SEQ ID NO: 61 - protein, 421 aa, artificial sequence
  synthetic peptide
LDITSHHHHHHDDLDAVRIKVDTVNAKPGDTVRIPVRFSGIPSKGIANCDFVYSYDPNVLEIIEIEPGDIIVDPNPDKSFDTAVYPDRKIIVFLFAEDSGTGAYAITKDGVFATIVAKVKEGAPNGLSVIKFVEVGGFANNDLVEQKTQFFDGGVNVGDTTEPATPTTPVTTPTTTDDLDAASKTSRPTAPSSGRGGNYPVQQIGGNYVHLPLSPRTLNAWVKLIEEKKFGAEVVPGFQALSEGCTPYDINQMLNCVGDHQAAMQIIRDIINEEAADWDLQHPQPAPQQGQLREPSGSDIAGTTSSVDEQIQWMYRQQNPIPVGNIYRRWIQLGLQKCVRMYNPTNILDVKQGPKEPFQSYVDRFYKSLRAEQTDAAVKNWMTQTLLIQNANPDCKLVLKGLGVNPTLEEMLTACQGVGAS

SEQ ID NO: 62 - protein, 390 aa, artificial sequence
  synthetic peptide
LDITSHHHHHHDDLDAVRIKVDTVNAKPGDTVRIPVRFSGIPSKGIANCDFVYSYDPNVLEIIEIEPGDIIVDPNPDKSFDTAVYPDRKIIVFLFAEDSGTGAYAITKDGVFATIVAKVKEGAPNGLSVIKFVEVGGFANNDLVEQKTQFFDGGVNVGDTTEPATPTTPVTTPTTTDDLDAASLGEVEDGYSQSPGGLDKGLSSLSCEGQKYNQGQYMNTPWRNPAEEREKLAYRKQNMDDIDEEDDDLVGVSVRPKVPLRTMSYKLAIDMSHFIKEKGGLEGIYYSARRHRILDIYLEKEEGIIPDWQDYTSGPGIRYPKTFGWLWKLVPVNVSDEAQEDEEHYLMHPAQTSQWDDPWGEVLAWKFDPTLAYTYEAYVRYPEEFGSKAS

SEQ ID NO: 63 - protein, 314 aa, artificial sequence
  synthetic peptide
LDITSHHHHHHDDLDAVRIKVDTVNAKPGDTVRIPVRFSGIPSKGIANCDFVYSYDPNVLEIIEIEPGDIIVDPNPDKSFDTAVYPDRKIIVFLFAEDSGTGAYAITKDGVFATIVAKVKEGAPNGLSVIKFVEVGGFANNDLVEQKTQFFDGGVNVGDTTEPATPTTPVTTPTTTDDLDAASAQSRTLLAGIVQQQQQLLDVVKRQQELLRLTVWGTKNLQTRVTAIEKYLKDQAQLNAWGCAFRQVCHTTVPWPNASLTPKWNNETWQEWERKVDFLEENITALLEEAQIQQEKNMYELQKLNSHHHHHHAS

SEQ ID NO: 64 - protein, 295 aa, artificial sequence
  synthetic peptide
MGSSHHHHHHSSGLVPRGSHMASMDLDAVRIKVDTVNAKPGDTVNIPVRFSGIPSKGIANCDFVYSYDPNVLEIIEIKPGELIVDPNPTKSFDTAVYPDRKMIVFLFAEDSGTGAYAITKDGVFATIVAKVKEGAPNGLSVIKFVEVGGFANNDLVEQKTQFFDGGVNVGDTTEPATPTTPVTTPTTTDDLDAASMAEMKTDAATLAQEAGNFERISGDLKTQIDQVESTAGSLQGQWRGAAGTAAQAAVVRFQEAANKQKQELDEISTNIRQAGVQYSRADEEQQQALSSQMGF

SEQ ID NO: 65 - protein, 550 aa, artificial sequence
  synthetic peptide
MGSSHHHHHHSSGLVPRGSHMASMDLDAVRIKVDTVNAKPGDTVNIPVRFSGIPSKGIANCDFVYSYDPNVLEIIEIKPGELIVDPNPTKSFDTAVYPDRKMIVFLFAEDSGTGAYAITKDGVFATIVAKVKEGAPNGLSVIKFVEVGGFANNDLVEQKTQFFDGGVNVGDTTEPATPTTPVTTPTTTDDLDAASMSNSRRRSLRWSWLLSVLAAVGLGLATAPAQAAPPALSQDRFADFPALPLDPSAMVAQVGPQVVNINTKLGYNNAVGAGTGIVIDPNGVVLTNNHVIAGATDINAFSVGSGQTYGVDVVGYDRTQDVAVLQLRGAGGLPSAAIGGGVAVGEPVVAMGNSGGQGGTPRAVPGRVVALGQTVQASDSLTGAEETLNGLIQFDAAIQPGDSGGPVVNGLGQVVGMNTAASDNFQLSQGGQGFAIPIGQAMAIAGQIRSGGGSPTVHIGPTAFLGLGVVDNNGNGARVQRVVGSAPAASLGISTGDVITAVDGAPINSATAMADALNGHHPGDVISVTWQTKSGGTRTGNVTLAEGPPA

SEQ ID NO: 66 - protein, 887 aa, artificial sequence
  synthetic peptide
MGSSHHHHHHSSGLVPRGSHMASMDLDAVRIKVDTVNAKPGDTVNIPVRFSGIPSKGIANCDFVYSYDPNVLEIIEIKPGELIVDPNPTKSFDTAVYPDRKMIVFLFAEDSGTGAYAITKDGVFATIVAKVKEGAPNGLSVIKFVEVGGFANNDLVEQKTQFFDGGVNVGDTTEPATPTTPVTTPTTTDDLDAASVGVSWPAKVRRRDGTLVPFDIARIEAAVTRAAREVACDDPDMPGTVAKAVADALGRGIAPVEDIQDCVEARLGEAGLDDVARVYIIYRQRRAELRTAKALLGVRDELKLSLAAVTVLRERYLLHDEQGRPAESTGELMDRSARCVAAAEDQYEPGSSRRWAERFATLLRNLEFLPNSPTLMNSGTDLGLLAGCFVLPIEDSLQSIFATLGQAAELQRAGGGTGYAFSHLRPAGDRVASTGGTASGPVSFLRLYDSAAGVVSMGGRRRGACMAVLDVSHPDICDFVTAKAESPSELPHFNLSVGVTDAFLRAVERNGLHRLVNPRTGKIVARMPAAELFDAICKAAHAGGDPGLVFLDTINRANPVPGRGRIEATNPCGEVPLLPYESCNLGSINLARMLADGRVDWDRLEEVAGVAVRFLDDVIDVSRYPFPELGEAARATRKIGLGVMGLAELLAALGIPYDSEEAVRLATRLMRRIQQAAHTASRRLAEERGAFPAFTDSRFARSGPRRNAQVTSVAPTGTISLIAGTTAGIEPMFAIAFTRAIVGRHLLEVNPCFDRLARDRGFYRDELIAEIAQRGGVRGYPRLPAEVRAAFPTAAEIAPQWHLRMQAAVQRHVEAAVSKTVNLPATATVDDVRAIYVAAWKAKVKGITVYRYGSREGQVLSYAAPKPLLAQADTEFSGGCAGRSCEF

SEQ ID NO: 67 - protein, 763 aa, artificial sequence
  synthetic peptide
MNRLTSSLLLLIVPAYVLSQVTLKESGPGILQPSQTLSLTCSFSGFSLSTSGMGLSWIRQPSGKGLEWLAHIYWDDDKRYNPSLKSRLTISKDTSSNQVFLKITIVDTADAATYYCARSSHYYGYGYGGYFDVWGAGTTVTVSSAKTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKASQTPTNTISVTPTNNSTPTNNSNPKPNPASMPKRSEYRQGTPNWVDLQTTDQSAAKKFYTSLFGWGYDDNPVPGGGGVYSMATLNGEAVAAIAPMPPGAPEGMPPIWNTYIAVDDVDAVVDKVVPGGGQVMMPAFDIGDAGRMSFITDPTGAAVGLWQANRHIGATLVNETGTLIWNELLTDKPDLALAFYEAVVGLTHSSMEIAAGQNYRVLKAGDAEVGGCMEPPMPGVPNHWHVYFAVDDADATAAKAAAAGGQVIAEPADIPSVGRFAVLSDPQGAIFSVLKPAPQQ

SEQ ID NO: 68 - protein, 400 aa, artificial sequence
  synthetic peptide
MGSSHHHHHHSSGLVPRGSHMASMDLDAVRIKVDTVNAKPGDTVNIPVRFSGIPSKGIANCDFVYSYDPNVLEIIEIKPGELIVDPNPTKSFDTAVYPDRKMIVFLFAEDSGTGAYAITKDGVFATIVAKVKEGAPNGLSVIKFVEVGGFANNDLVEQKTQFFDGGVNVGDTTEPATPTTPVTTPTTTDDLDAASMTGPTTDADAAVPRRVLIAEDEALIRMDLAEMLREEGYEIVGEAGDGQEAVELAELHKPDLVIMDVKMPRRDGIDAASEIASKRIAPIVVLTAFSQRDLVERARDAGAMAYLVKPFSISDLIPAIELAVSRFREITALEGEVATLSERLETRKLVERAKGLLQTKHGMTEPDAFKWIQRAAMDRRTTMKRVAEVVLETLGTPKDT

SEQ ID NO: 69 - protein, 388 aa, artificial sequence
  synthetic peptide
MGSSHHHHHHSSGLVPRGSHMASMDLDAVRIKVDTVNAKPGDTVNIPVRFSGIPSKGIANCDFVYSYDPNVLEIIEIKPGELIVDPNPTKSFDTAVYPDRKMIVFLFAEDSGTGAYAITKDGVFATIVAKVKEGAPNGLSVIKFVEVGGFANNDLVEQKTQFFDGGVNVGDTTEPATPTTPVTTPTTTDDLDAASMKVKNTIAATSFAAAGLAALAVAVSPPAAAGDLVGPGCAEYAAANPTGPASVQGMSQDPVAVAASNNPELTTLTAALSGQLNPQVNLVDTLNSGQYTVFAPTNAAFSKLPASTIDELKTNSSLLTSILTYHVVAGQTSPANVVGTRQTLQGASVTVTGQGNSLKVGNADVVCGGVSTANATVYMIDSVLMPPA

SEQ ID NO: 70 - protein, 554 aa, artificial sequence
  synthetic peptide
MGSSHHHHHHSSGLVPRGSHMASMDLDAVRIKVDTVNAKPGDTVNIPVRFSGIPSKGIANCDFVYSYDPNVLEIIEIKPGELIVDPNPTKSFDTAVYPDRKMIVFLFAEDSGTGAYAITKDGVFATIVAKVKEGAPNGLSVIKFVEVGGFANNDLVEQKTQFFDGGVNVGDTTEPATPTTPVTTPTTTDDLDAASMRSTVAVAVAAAVIAASSGCGSDQPAHKASQSMITPTTQIAGAGVLGNDRKPDESCARAAAAADPGPPTRPAHNAAGVSPEMVQVPAEAQRIVVLSGDQLDALCALGLQSRIVAAALPNSSSSQPSYLGTTVHDLPGVGTRSAPDLRAIAAAHPDLILGSQGLTPQLYPQLAAIAPTVFTAAPGADWENNLRGVGAATARIAAVDALITGFAEHATQVGTKHDATHFQASIVQLTANTMRVYGANNFPASVLSAVGVDRPPSQRFTDKAYIEIGTTAADLAKSPDFSAADADIVYLSCASEAAAERAAVILDSDPWRKLSANRDNRVFVVNDQVWQTGEGMVAARGIVDDLRWVDAPIN

SEQ ID NO: 71 - protein, 588 aa, artificial sequence
  synthetic peptide
MGSSHHHHHHSSGLVPRGSHMASMDLDAVRIKVDTVNAKPGDTVNIPVRFSGIPSKGIANCDFVYSYDPNVLEIIEIKPGELIVDPNPTKSFDTAVYPDRKMIVFLFAEDSGTGAYAITKDGVFATIVAKVKEGAPNGLSVIKFVEVGGFANNDLVEQKTQFFDGGVNVGDTTEPATPTTPVTTPTTTDDLDAASVVDFGALPPEINSARMYAGPGSASLVAAAKMWDSVASDLFSAASAFQSVVWGLTVGSWIGSSAGLMAAAASPYVAWMSVTAGQAQLTAAQVRVAAAAYETAYRLTVPPPVIAENRTELMTLTATNLLGQNTPAIEANQAAYSQMWGQDAEAMYGYAATAATATEALLPFEDAPLITNPGGLLEQAVAVEEAIDTAAANQLMNNVPQALQQLAQPAQGVVPSSKLGGLWTAVSPHLSPLSNVSSIANNHMSMMGTGVSMTNTLHSMLKGLAPAAAQAVETAAENGVWAMSSLGSQLGSSLGSSGLGAGVAANLGRAASVGSLSVPPAWAAANQAVTPAARALPLTSLTSAAQTAPGHMLGGLPLGHSVNAGSGINNALRVPARAYAIPRTPAAG

SEQ ID NO: 72 - protein, 479 aa, artificial sequence
  synthetic peptide
MGSSHHHHHHSSGLVPRGSHMASMDLDAVRIKVDTVNAKPGDTVNIPVRFSGIPSKGIANCDFVYSYDPNVLEIIEIKPGELIVDPNPTKSFDTAVYPDRKMIVFLFAEDSGTGAYAITKDGVFATIVAKVKEGAPNGLSVIKFVEVGGFANNDLVEQKTQFFDGGVNVGDTTEPATPTTPVTTPTTTDDLDAASSRPGLPVEYLQVPSPSMGRDIKVQFQSGGNNSPAVYLLDGLRAQDDYNGWDINTPAFEWYYQSGLSIVMPVGGQSSFYSDWYSPACGKAGCQTYKWETFLTSELPQWLSANRAVKPTGSAAIGLSMAGSSAMILAAYHPQQFIYAGSLSALLDPSQGMGPSLIGLAMGDAGGYKAADMWGPSSDPAWERNDPTQQIPKLVANNTRLWVYCGNGTPNELGGANIPAEFLENFVRSSNLKFQDAYNAAGGHNAVFNFPPNGTHSWEYWGAQLNAMKGDLQSSLGAG

SEQ ID NO: 73 - protein, 917 aa, artificial sequence
  synthetic peptide
MGSSHHHHHHSSGLVPRGSHMASMDLDAVRIKVDTVNAKPGDTVNIPVRFSGIPSKGIANCDFVYSYDPNVLEIIEIKPGELIVDPNPTKSFDTAVYPDRKMIVFLFAEDSGTGAYAITKDGVFATIVAKVKEGAPNGLSVIKFVEVGGFANNDLVEQKTQFFDGGVNVGDTTEPATPTTPVTTPTTTDDLDAASTAASDNFQLSQGGQGFAIPIGQAMAIAGQIRSGGGSPTVHIGPTAFLGLGVVDNNGNGARVQRVVGSAPAASLGISTGDVITAVDGAPINSATAMADALNGHHPGDVISVTWQTKSGGTRTGNVTLAEGPPAEFMVDFGALPPEINSARMYAGPGSASLVAAAQMWDSVASDLFSAASAFQSVVWGLTVGSWIGSSAGLMVAAASPYVAWMSVTAGQAELTAAQVRVAAAAYETAYGLTVPPPVIAENRAELMILIATNLLGQNTPAIAVNEAEYGEMWAQDAAAMFGYAAATATATATLLPFEEAPEMTSAGGLLEQAAAVEEASDTAAANQLMNNVPQALQQLAQPTQGTTPSSKLGGLWKTVSPHRSPISNMVSMANNHMSMTNSGVSMTNTLSSMLKGFAPAAAAQAVQTAAQNGVRAMSSLGSSLGSSGLGGGVAANLGRAASVGSLSVPQAWAAANQAVTPAARALPLTSLTSAAERGPGQMLGGLPVGQMGARAGGGLSGVLRVPPRPYVMPHSPAAGDIAPPALSQDRFADFPALPLDPSAMVAQVGPQVVNINTKLGYNNAVGAGTGIVIDPNGVVLTNNHVIAGATDINAFSVGSGQTYGVDVVGYDRTQDVAVLQLRGAGGLPSAAIGGGVAVGEPVVAMGNSGGQGGTPRAVPGRVVALGQTVQASDSLTGAEETLNGLIQFDAAIQPGDSGGPVVNGLGQVVGMNTAAS

SEQ ID NO: 74 - protein, 291 aa, artificial sequence
  synthetic peptide
MGSSHHHHHHSSGLVPRGSHMASMDLDAVRIKVDTVNAKPGDTVNIPVRFSGIPSKGIANCDFVYSYDPNVLEIIEIKPGELIVDPNPTKSFDTAVYPDRKMIVFLFAEDSGTGAYAITKDGVFATIVAKVKEGAPNGLSVIKFVEVGGFANNDLVEQKTQFFDGGVNVGDTTEPATPTTPVTTPTTTDDLDAASMSQIMYNYPAMLGHAGDMAGYAGTLQSLGAEIAVEQAALQSAWQGDTGITYQAWQAQWNQAMEDLVRAYHAMSSTHEANTMAMMARDTAEAAKWGG

SEQ ID NO: 75 - protein, 400 aa, artificial sequence
  synthetic peptide
MGSSHHHHHHSSGLVPRGSHMASMDLDAVRIKVDTVNAKPGDTVNIPVRFSGIPSKGIANCDFVYSYDPNVLEIIEIKPGELIVDPNPTKSFDTAVYPDRKMIVFLFAEDSGTGAYAITKDGVFATIVAKVKEGAPNGLSVIKFVEVGGFANNDLVEQKTQFFDGGVNVGDTTEPATPTTPVTTPTTTDDLDAASCSCMAENSNIDDIKAPLLAALGAADLALATVNELITNLRERAEETRTDTRSRVEESRARLTKLQEDLPEQLTELREKFTAEELRKAAEGYLEAATSRYNELVERGEAALERLRSQQSFEEVSARAEGYVDQAVELTQEALGTVASQTRAVGERAAKLVGIELPKKAAPAKKAAPAKKAAPAKKAAAKKAPAKKAAAKKVTQKCSC

SEQ ID NO: 76 - protein, 403 aa, artificial sequence
  synthetic peptide
MGSSHHHHHHSSGLVPRGSHMASMDLDAVRIKVDTVNAKPGDTVNIPVRFSGIPSKGIANCDFVYSYDPNVLEIIEIKPGELIVDPNPTKSFDTAVYPDRKMIVFLFAEDSGTGAYAITKDGVFATIVAKVKEGAPNGLSVIKFVEVGGFANNDLVEQKTQFFDGGVNVGDTTEPATPTTPVTTPTTTDDLDAASAAPKTYCEELKGTDTGQACQIQMSDPAYNINISLPSYYPDQKSLENYIAQTRDKFLSAATSSTPREAPYELNITSATYQSAIPPRGTQAVVLKVYQNAGGTHPTTTYKAFDWDQAYRKPITYDTLWQADTDPLPVVFPIVQGELSKQTGQQVSIAPNAGLDPVNYQNFAVTNDGVIFFFNPGELLPEAAGPTQVLVPRSAIDSMLAEF